(19) United States
(12) Patent Application Publication
(10) Pub. No.: US 2022/0356482 A1
(43) Pub. Date: Nov. 10, 2022

(54) Controlling Plant Flowering

(71) Applicant: COMMONWEALTH SCIENTIFIC AND INDUSTRIAL RESEARCH ORGANISATION, Acton, ACT (AU)

(72) Inventors: Mark THOMAS, Acton, ACT (AU); Patricia CORENA, Acton, ACT (AU); Lekha SREEKANTAN, Acton, ACT (AU); Donald MACKENZIE, Acton, ACT (AU); Matthew SMITH, Acton, ACT (AU)

(73) Assignee: COMMONWEALTH SCIENTIFIC AND INDUSTRIAL RESEARCH ORGANISATION, Acton, ACT (AU)

(21) Appl. No.: 17/623,431

(22) PCT Filed: Jun. 29, 2020
(86) PCT No.: PCT/AU2020/050673

§ 371 Date: Dec. 28, 2021

(30) Foreign Application Priority Data
Jun. 28, 2019 (AU) 2019902304
Jul. 12, 2019 (AU) 2019902483

(51) Int. Cl. C12N 15/82; C07K 14/415

(52) U.S. Cl. CPC C12N 15/827; C07K 14/415; C12N 15/8218

(57) ABSTRACT

The present disclosure relates to plants and plant parts having an altered level of Flower Sex (FSL) polypeptide activity and methods of controlling plant flower sex phenotype based on altered FSL polypeptide activity and/or FSL locus genotype. Also provided are novel plants which produced stenospermocarpic and/or parthenocarpic seedless fruit, and methods of producing same.

### RELATED APPLICATION DATA

This application claims the right of priority to Australian Provisional Application No. 2019902304, filed 28 Jun. 2019, and Australian Provisional Application No. 2019902483, filed 12 Jul. 2019, the full contents on each of which is incorporated by reference herein in its entirety.

### FIELD OF THE INVENTION

The present disclosure relates to plants and plant parts having an altered level of Flower Sex (FSL) polypeptide activity and methods of controlling plant flower sex phenotype based on altered FSL polypeptide activity and/or FSL locus genotype. Also provided are novel plants which produced stenospermocarpic and/or parthenocarpic seedless fruit, and methods of producing same.

### BACKGROUND OF THE INVENTION

### Flower Sex

Wild grapevine plants, sometimes referred to as Vitis sylvestris, are dioecious, meaning the plants have either male or female flowers. Wild plants flower once they reach the canopy top and are exposed to high light, producing a large number of small bunches of flowers (Carmona et al., 2008). Berries produced by wild grapevine female plants are small and in small bunches. Unisexual flowers produced by Vitis species still possess rudimentary organs of the opposite sex. Cultivated grapevine plants are hermaphrodites. Commercial vineyards have plants with hermaphroditic flowers where autogamy (self-fertilization) is thought to be the major route for pollination.

The sex of the flowers is identified by observation of physical characteristics which requires a mature plant that is flowering and even then with matured flowers. Male flowers have erect stamens, viable pollen and an underdeveloped small non-functional carpel. Hermaphrodite flowers have erect stamens, viable pollen and a functional carpel. The female flower is characterized by a functional carpel and reflexed stamens and infertile pollen that does not germinate (Carmona et al 2008). Dioecious plants with female flowers only are typically of lesser commercial value for fruit production because they require a male or hermaphrodite plant nearby to provide pollen to set fruit (Battilana et al., 2013). Flower types follow a bisexual development pattern during the early stages of floral development, with unisexuality arising through organ abortion in late stage, when the maturity of all flower organs takes place (Pannell, 2017), this seems to apply across the flowering plants and is postulated to have evolved with the origin of flowers (Chanderbali et al., 2010).

Flower sex remains of interest commercially as it can be a problem for breeding, as well as for production and crop yield (e.g. seed collection or fruit size, fruit yield per plant). In some species the ratio of the female to male flowers has been a cause of low yield (Mao et al 2017—in Vernicia fordii (tung oil tree)). Maize (Zea mays), cucumber (Cucumis sativus) and melon (Cucumis melo) are monoecious plants that have been undergone significant study and development (Tanurdzic and Banks 2004) to become significant crops for food and feed. Dioecious plants with male and female flowers on separate plants, include white campion (Silene latifolia), papaya (Carica papava), hemp (Cannabis saliva), and annual mercury (Mercurialis annua) (Mao et al 2017).

Plant flower sex can be influenced or manipulated by environmental conditions, genetic mutation or hormone application, as such the sexual identity of a plant is considered quantitative (Pannell 2017). In the Cucurbitaceae family, sex expression is controlled by a network of genetic, hormonal and environmental factors. Cucumbers (Cucumis sativus) are one crop that have been bred to be gynoecious to increase productivity through production of only female flowers. The sexual expression is thought to be controlled by an F locus, which regulates female flower expression, and an M locus considered to regulate bisexual flower expression (Yamasaki et al 2001). The sexual expression is capable of being modulated by plant hormones such as ethylene, and environmental stimuli. In watermelon, gynoecious (gy), andromonoecious (a), and trimonoecious (tm) loci control the inheritance of sexual forms (Ji et al., 2015). At the genetic level, the sex determination of cucumber, melon and watermelon is controlled by combinations of three pairs of genes. Monoecious cucumber is controlled by a 1-aminocyclopropane-1-carboxylate synthase (ACS) gene that is specifically expressed in carpels and is involved in the arrest of stamen development in female flowers (Manzano et al., 2011). This gene and members of its family similar control flower sex in watermelon having roles in rate-limiting enzyme in ethylene biosynthesis (Ji et al., 2016), loss of function resulting in bisexual flowers (CsACS11/CsACS2) or promotion of female flowers (CsACS1G) and interaction with transcription factors (CmWIP1) to influence the plant to express gynoecious or hermaphroditic flowers (Jie et al 2017).

The ethylene biosynthesis enzyme 1-aminocyclopropane-1-carboxylate synthase (ACS) has a key role in influencing female flower expression in monoecious, andromonoecious and gynomecious cucumber plants (Yamasaki et al 2000; Yamasaki et al 2001). CS-ACS2 was found to only be expressed in gynoecious cucumber plants and was responsible for causing the higher levels of ethylene production and regulated by the F locus (Yamasaki et al 2001). Ethylene is the primary hormone promoting female flower development in melon and cucumber, whereas gibberellins have opposite effects in these plants (Yamasaki et al 2005). In contrast to the feminising effect on the cucurbit species (cucumber, melon and squash) ethylene had a masculinizing effect in watermelon (Manzano et al., 2011). Jie et al (2017) further demonstrate the effects of ethylene, and ethylene competitors, gibberellin and silver nitrate, in the different genetic backgrounds of watermelon compared to the published responses in cucumber. These results suggest the hormonal production and response has a significant interaction and reliance on the plant genetics but this remains to be fully elucidated (Jie et al 2017).

Controlling or altering flower sex has practical applications in breeding and developing hybrids or populations. Since Peterson and Anhder (1960) reported the masculinizing effect of Gibberellin in cucumbers it has been widely used to maintain gynoecious breeding lines and to produce seed in all female cucumber cultivars. Such gynoecious inbred lines reduce breeding and development costs, can sustain or provide yield improvements and seed quality.

Although the determinants of sexual phenotype are diverse, it remains unclear if the changes in expression of these genes are a cause or a consequence of organ sex determination. Therefore there is a need to show whether the downstream regulatory genes that specific male of female development are master sex controlling genes. In Vitis, hormones can modify flower sexual development, and cytokinins have been shown to play a major role in the process (Negi and Olmo, 1966, 1971; Zhang et al 2013).

In grapevines of Vitis sp., the location of the flower sex locus to linkage group 2 (LG2) was previously proposed by Dalbo, et al (2000) and Riaz et al (2006) to be located on chromosome 2 close to the genetic marker VviS3. Confirmed in the microvine and picovine population by Chaib et al (2010). Fetcher e al., (2012) identified VviAPRT, which encodes adenine phosphoribosyiltransferase, as the marker to discriminate female from male/hermaphrodite plants. Fetcher et al., (2012) predicted eleven genes and reported that an adenine phophoribosyltransferase (APRT, now referred to as APRT3) has a key role in determining the grapevine flower sex and its expression identifies with female flowers. However Coito et al (2017) found APRT3 distinguished male from female and hermaphrodite plants proposing a model that includes a third unknown gene. Gibberellin (GA) is regarded as ethylene competitor to promote the production of male flowers and inhibit the development of female flowers in cucumber (Friedlander et al., 1977).

Although a number of candidate genes have been proposed as the genetic controller of flower sex in grapevines, the gene(s) that control flower sex remain unknown.

### Seedless Grapes

Table grape varieties that are “seedless” produce seedless fruit due to stenospermocarpy where the flower is fertilised and the seed starts to develop but stops development at an early stage (i.e., aborts) leaving only a seed trace in the fruit. A mutated locus of the Vitis vinifera MADS-box protein 5 (VvMADS5, also known as VviAGL11) gene (in either the heterozygous or homozygous state) is associated with stenospermocarpy (SDL1) in grapevine. The mutation has a G to T substitution at 590 bp of the coding sequence resulting in an Arg-197Leu substitution and has recently been hypothesised to be the associated with the stenospermocarpic seedlessness phenotype (Royo et al 2018).

Although the genetic control of stenospermocarpic seedless grapes that produce a seed trace following pollination is thought to be due to a mutation (SDL1) in the MADS5 gene, the genetic control of parthenocarpic seedless berry development in grapevines is not known or understood.

### Microvines

The development of dwarf grape plants with a rapid flowering phenotype, referred to as microvines, were previously described by the inventors (Boss and Thomas, Nature, 2002) and have a SNP in the grapevine GA insensitive gene (VvGAI1). The single nucleotide difference from a T to an A between VvGAI1 and Vvgai1 is in the translated region at position 231. The point mutation present in the VvGAI1 allele converts a leucine residue of the conserved DELLA domain into histidine. The mutated gene Gibberellic Acid Insensitive gene is dominant (in either the heterozygous (GAI1/gai1) or homozygous state (GAI1/GAI1) causes a dwarf stature and rapid flowering phenotype.

### Grapevine Breeding and Improvement

For grapevine improvement, there is a need to modify and be able to control the flower sex for breeding purposes to combine or maintain favourable phenotypic traits. For table grape breeding and production it is desirable to produce true parthenocarpic seedless fruit that do not produce seed traces. For urban/indoor farming and covered cropping it is desirable to have dwarf table grape selections that can grow at high density and produce fresh fruit that are seedless.

### SUMMARY OF THE INVENTION

The present disclosure is based, at least in part, on the surprising finding by the inventors that a locus, which has been termed the Flower Sex (FSL) locus, is responsible for flower sex in angiosperms, such as grapevines, and that different FSL locus genotypes and polypeptides expressed therefrom can be used to determine, control and/or select flower sex phenotype i.e., female, male or hermaphrodite flower phenotypes respectively. The inventors have characterized the FSL locus responsible for male organ development which behaves similarly to Sp in the Oberles 1938 model for flower sex determination whereby in a Vitis sp. It is dominant in both male and hermaphrodites. In the female, the locus is recessive and non-functional. The inventors have also demonstrated 100% concordance between female (fsl/fsl) and hermaphrodite (FSL/fsl or FSL/FSL) genotypes at a single nucleotide polymorphism (SNP) within a plant AT-rich sequence- and zinc-binding (PLATZ) domain of the FSL locus and the respective flower sex phenotype. vinifera

Thus, in a first aspect, the present disclosure provides a plant or part thereof having an altered level of flower sex (FSL) polypeptide activity compared to a corresponding plant or part thereof having a FSL locus genotype which confers a male or hermaphrodite flower phenotype.

In one example, the plant or part thereof has an altered level of FSL polypeptide activity compared to a corresponding plant or part thereof having a FSL locus genotype which confers a hermaphrodite flower phenotype. In one example, the FSL locus genotype which confers a hermaphrodite flower phenotype comprises a hermaphrodite allele of the FSL locus. In one example, the hermaphrodite allele of the FSL locus encodes a FSL polypeptide comprising an amino acid sequence set forth in SEQ ID NO:1, a biologically active fragment thereof, or an amino acid sequence which is at least 40% identical to the sequence set forth in SEQ ID NO:1. For example, the FSL polypeptide encoded by the hermaphrodite allele of the FSL locus may comprise an amino acid sequence which is at least 50%, at least 60%, at least 70%, at least 80%, at least 85%, at least 90%, at least 95%, or at least 96% identical to the sequence set forth in SEQ ID NO:1. For example, the FSL polypeptide may comprise an amino acid sequence which is at least 97%, at least 98%, or at least 99% identical to the sequence set forth in SEQ ID NO:1. In each of the foregoing examples describing exemplary FSL polypeptides comprising amino acid sequences having a level of identity to the sequence set forth in SEQ ID NO: 1, the FSL polypeptides may be orthologues of the FSL polypeptide set forth in SEQ ID NO:1. In one particular example, the FSL polypeptide comprises an amino acid sequence set forth in SEQ ID NO:1.

In one example, the plant or part thereof has an altered level of FSL polypeptide activity compared to a corresponding plant or part thereof having a FSL locus genotype which confers a male flower phenotype. In one example, the FSL locus genotype which confers a male flower phenotype comprises a male allele of the FSL locus. In one example, the hermaphrodite allele of the FSL locus encodes a FSL polypeptide comprising an amino acid sequence set forth in SEQ ID NO: 3, a biologically active fragment thereof, or an amino acid sequence which is at least 40% identical to the sequence set forth in SEQ ID NO: 3. For example, the FSL polypeptide of the male allele of the FSL locus may comprise an amino acid sequence which is at least 50%, at least 60%, at least 70%, at least 80%, at least 85%, at least 90%, at least 95%, or at least 96% identical to the sequence set forth in SEQ ID NO: 3. For example, the FSL polypeptide may comprise an amino acid sequence which is at least 97%, at least 98% or at least 99% identical to the sequence set forth in SEQ ID NO: 3. In each of the foregoing example describing exemplary FSL polypeptides comprising amino acid sequences having a level of identity to the sequence set forth in SEQ ID NO: 3, the FSL polypeptides may be orthologues of the FSL polypeptide set forth in SEQ ID NO: 3. In one particular example, the FSL polypeptide comprises an amino acid sequence set forth in SEQ ID NO: 3.

In one example, the plant or part thereof comprises an FSL locus comprising a polynucleotide sequence encoding the FSL polypeptide, wherein the polynucleotide sequence is modified relative to a corresponding polynucleotide sequence of a wildtype FSL locus allele which confers a male or hermaphrodite flower phenotype when expressed. For example, the polynucleotide sequence encoding the FSL polypeptide may be modified relative to a corresponding polynucleotide sequence of a wildtype hermaphrodite allele of the FSL locus. For example, the polynucleotide sequence encoding the FSL polypeptide may be modified relative to a corresponding polynucleotide sequence of a wildtype male allele of the FSL locus. In some examples, a region of the polynucleotide sequence encoding a plant AT-rich sequence- and zinc-binding (PLATZ) domain of the FSL locus may be modified e.g., relative to a polynucleotide sequence encoding a corresponding wildtype PLATZ domain. In one example, the polynucleotide sequence encoding the wildtype PLATZ domain encodes an amino acid sequence set forth from residue 26 to residue 75 of the sequence set forth in SEQ ID NO: 1. In one example, the polynucleotide sequence encoding the FSL polypeptide comprises one or more nucleotide additions, deletions or substitutions relative to the corresponding polynucleotide sequence of a wildtype FSL locus allele which confers a male or hermaphrodite flower phenotype when expressed e.g., one or more nucleotide additions, deletions or substitutions in the sequence encoding the PLATZ domain. For example, the polynucleotide sequence encoding the FSL polypeptide may comprise one or more (e.g., 1, 2, 3, 4, 5, 6, 7, 8, 9, 10 or more) nucleotide additions, deletions or substitutions between positions 153 and 189, such as between positions 155 and 159, relative to the ORF sequence set forth in SEQ ID NO: 6 or 7 (or at one or more corresponding nucleotide positions of the corresponding genomic sequence). For example, the polynucleotide sequence encoding the FSL polypeptide may have one or more (e.g., 1, 2, 3, 4, 5, 6, 7, 8, 9, 10 or more) nucleotides deleted between positions 153 and 189 relative to the ORF sequence set forth in SEQ ID NO: 6 or 7 (or at one or more corresponding nucleotide positions of the corresponding genomic sequence). For example, the polynucleotide sequence encoding the FSL polypeptide may have one or more T's (e.g., T, TT or TTT) deleted between positions 155 and 159 relative to the ORF sequence set forth in SEQ ID NO: 6 or 7 (or one or more T's deleted at one or more corresponding nucleotide positions of the corresponding genomic sequence). For example, the polynucleotide sequence encoding the FSL polypeptide may have one or more T's (e.g., T, TT or TTT) added between positions 155 and 159 relative to the ORF sequence set forth in SEQ ID NO: 6 or 7 (or one or more T's added at one or more corresponding nucleotide positions of the corresponding genomic sequence). In some examples, the polynucleotide sequence encoding the FSL polypeptide has been gene-edited.

In some examples, the FSL polypeptide encoded by the modified polynucleotide sequence comprises one or more amino acid additions, deletions or substitutions relative to the FSL polypeptide encoded by the corresponding wildtype FSL locus allele (e.g., as a result of one or more one or more nucleotide additions, deletions or substitutions to the encoding polynucleotide sequence). For example, the plant or part thereof may comprise a FSL polypeptide comprising one or more amino acid additions, deletions or substitutions in the PLATZ domain relative to the corresponding amino acid sequence encoded by the corresponding wildtype FSL locus allele. In one example, the PLATZ domain encoded by the corresponding wildtype FSL locus allele comprise the amino acid sequence set forth from residue 26 to residue 75 of the sequence set forth in SEQ ID NO: 1. In some examples, the FSL polypeptide is truncated. In some examples, the FSL polypeptide or a domain thereof e.g., the PLATZ domain, is absent from the plant or part thereof.

In some examples, the plant or part thereof comprises an RNA interference (RNAi) agent which targets a messenger RNA (mRNA) of the FSL locus, thereby reducing FSL polypeptide activity in the plant or part thereof compared to a corresponding plant or part thereof which does not comprise the RNAi agent. In accordance with examples in which the plant or plant part comprises an RNAi agent, the plant or part thereof may be transfected with and/or have incorporated into its genome a construct for expressing the RNAi agent e.g., an expression vector which expresses the RNAi agent. The RNAi agent may be any RNAi agent known in the art or described herein.

In one example, the corresponding wildtype FSL locus allele is a hermaphroditic allele of the FSL locus. In accordance with this example, the ORF of the corresponding wildtype FSL locus allele may comprises a polynucleotide sequence set forth in SEQ ID NO: 6, a sequence which is at least 60% identical thereto, or an orthologous sequence thereof corresponding to the species of plant. In one example, the ORF of the corresponding wildtype FSL locus allele comprises a sequence which is at least 65%, at least 70%, at least 75%, at least 80%, at least 85%, at least 90%, at least 95%, at least 96%, at least 97%, at least 98%, or at least 99% identical to the sequence set forth in SEQ ID NO: 6. In each of the foregoing examples describing an ORF of a corresponding wildtype FSL locus allele comprising a sequence which has a percentage level of identity relative to a sequence set forth in SEQ ID NOs: 6, the wildtype FSL locus allele may be an orthologue of the sequence set forth in SEQ ID NO: 6. In this regard, the sequence set forth in SEQ ID NO: 6 represents the ORF of the hermaphrodite allele of the FSL locus for Vitus vinifera. In one particular example, the ORF of the corresponding wildtype FSL locus allele comprises a sequence set forth in SEQ ID NO: 6.

In another example, the corresponding wildtype FSL locus allele is a male allele of the FSL locus. In accordance with this example, the ORF of the corresponding wildtype FSL locus allele may comprises a polynucleotide sequence set forth in SEQ ID NO: 7, a sequence which is at least 60% identical thereto, or an orthologous sequence thereof corresponding to the species of plant. In one example, the ORF of the corresponding wildtype FSL locus allele comprises a sequence which is at least 65%, at least 70%, at least 75%, at least 80%, at least 85%, at least 90%, at least 95%, at least 96%, at least 97%, at least 98%, or at least 99% identical to the sequence set forth in SEQ ID NO: 6. In each of the foregoing examples describing an ORF of a corresponding wildtype FSL locus allele comprising a sequence which has a percentage level of identity relative to a sequence set forth in SEQ ID NO: 7, the wildtype FSL locus allele may be an orthologue of the sequences set forth in SEQ ID NO: 7. In this regard, the sequence set forth in SEQ ID NO: 7 represents the ORF of the male allele of the FSL locus for Vitus vinifera. In one particular example, the ORF of the wildtype FSL locus comprises a sequence set forth in SEQ ID NO: 7.

In one example, the corresponding wildtype FSL locus allele comprises a polynucleotide sequence encoding a PLATZ domain comprising an amino acid sequence set forth from residue 26 to residue 75 of the sequence set forth in SEQ ID NO: 1 or an amino acid sequence which is at least 70% identical thereto. For example, the corresponding wildtype FSL locus allele may comprise a sequence encoding a PLATZ domain comprising an amino acid sequence which is at least 75%, at least 80%, at least 85%, at least 90%, at least 95%, at least 96% identical, at least 97% identical, at least 98% or at least 99% identical to the amino acid sequence set forth from residue 26 to residue 75 of the sequence set forth in SEQ ID NO: 1. In one particular example, the corresponding wildtype FSL locus allele comprises a sequence encoding a PLATZ domain comprising the amino acid sequence set forth from residue 26 to residue 75 of the sequence set forth in SEQ ID NO: 1.

In some examples, the FSL polypeptide activity is reduced in the plant or plant part relative a level of FSL polypeptide activity in a corresponding wildtype plant or part thereof. For example, FSL polypeptide activity in the plant or plant part may be reduced by at least 10% relative to a level of FSL polypeptide activity in a corresponding wildtype plant or part thereof. For example, FSL polypeptide activity in the plant or plant part may be reduced by at least 20%, at least 30%, at least 40%, at least 50%, at least 60%, at least 70%, at least 80%, at least 90%, at least 95% relative to a level of FSL polypeptide activity in a corresponding wildtype plant or part thereof.

In each of the foregoing examples, the FSL polypeptide activity may be reduced relative to a level of FSL polypeptide activity in a corresponding plant or part thereof comprising a male or hermaphrodite allele of the FSL locus. For example, the FSL polypeptide activity may be reduced relative to a level of FSL polypeptide activity in a corresponding hermaphroditic wildtype plant or part thereof, or relative to a level of FSL polypeptide activity in a corresponding male wildtype plant or part thereof.

In each of the foregoing examples describing a plant or plant part having a reduced level of FSL polypeptide activity, the reduced FSL polypeptide activity may be caused by a corresponding reduction in expression of FSL polypeptide relative to a level of expression in a corresponding wildtype plant or part thereof. Alternatively or in addition, the reduced FSL polypeptide activity may be caused by a corresponding reduction in expression of FSL locus mRNA relative to a level of expression in a corresponding wildtype plant or part thereof.

In some examples, FSL polypeptide activity is abrogated in the plant or plant part. For example, FSL polypeptide expression may be completely inhibited or the FSL locus encoding the FSL polypeptide may be knock-out in the plant or part thereof.

In one example, the altered activity of FSL polypeptide in the plant or part thereof causes a male reproductive part of a flower of the plant to be absent or non-functional. For example, a reduction in activity of FSL polypeptide as described herein may cause a male reproductive part of a flower of the plant or plant part to be absent or non-functional. In some examples, the male reproductive part of a flower is present but non-functional due to the altered e.g., reduced, activity of FSL polypeptide. A non-functional male reproductive part of a flower may be underdeveloped due to the altered e.g., reduced, activity of FSL polypeptide, causing it to be non-functional.

In some examples, the plant produces flowers which are male sterile.

The present disclosure also provides a plant or part thereof having a reduced level of FSL polypeptide activity which produces a phenotypically female flower, wherein the level of FSL polypeptide activity is reduced compared to a plant or plant part which produces a flower comprising functional male reproductive parts. In some examples the plant or plant part may be a plant or plant part having an altered level of FSL polypeptide activity as described herein e.g., an altered level of FSL polypeptide activity relative to corresponding plant or plant part comprising a male or hermaphrodite allele of the FSL locus. In some examples, the plant or plant part may comprise an FSL locus which is homozygous for a female allele (f/f) conferring a female flower phenotype. In some examples, the FSL locus (f/f) genotype is non-naturally occurring in the plant or plant part.

The present disclosure also provides a plant or part thereof which produces seedless fruit, said plant comprising:
(i) a polynucleotide that confers dwarf stature to a plant; and
(ii) a flower sex (FSL) locus which is homozygous for a female allele (f/f) conferring a female flower phenotype.

In one example, the ORF of the female allele of the FSL locus comprises a sequence set forth in SEQ ID NO: 5, or a sequence having at least 70% identity thereto provided that the nucleotide corresponding to position 621 of the sequence set forth in SEQ ID NO: 5 is a A. For example, the ORF of the female allele of the FSL locus may comprise a sequence having at least 75%, at least 80%, at least 85%, at least 90%, at least 95%, at least 96%, at least 97%, at least 98% or at least 99% identity to the sequence set forth in SEQ ID NO: 5 provided that the nucleotide corresponding to position 621 of the sequence set forth in SEQ ID NO: 5 is a A. In some examples, the ORF of the female allele of the FSL locus comprises the sequence set forth in SEQ ID NO: 5.

In each of the foregoing examples describing an ORF of a female allele of the FSL locus which has a percentage level of identity to the sequence set forth in SEQ ID NO: 5, the female allele of the FSL locus may be an orthologue of the sequence set forth in SEQ ID NO: 5 corresponding to the plant species.

The present disclosure also provides a plant or part thereof having an altered level of FSL polypeptide activity as described herein, wherein said plant comprises a polynucleotide that confers dwarf stature to the plant. A plant or plant part in accordance with this embodiment produces seedless fruit.

In each of the foregoing examples describing plants or plant parts which produce seedless fruit, the polynucleotide that confers dwarf stature is altered relative to the corresponding wildtype polynucleotide sequence.

In one example, the polynucleotide that confers dwarf stature is a variant of the gibberellic acid insensitive (GAI1) gene or a fragment thereof. The variant of the GAI1 gene encodes a variant GAI1 protein. In one example, the variant of the GAI1 gene or fragment thereof that confers dwarf stature to the plant comprises one or more mutations in a region encoding the DELLA domain. For example, the one or more mutations in the region encoding the DELLA domain of the GAI1 protein may alter gibberellic acid (GA) response properties of the plant or plant part. The one or more mutations may be selected from amino acid substitutions, deletions or additions. The one or more mutations in the DELLA domain may prevent the plant or plant part from responding to GA signalling. Accordingly, in some examples, the plant or plant part comprising a variant of the GAI1 gene or a fragment thereof does not respond, or responds poorly, to GA signalling. In one example, the variant GAI1 protein comprises a sequence set forth in SEQ ID NO: 8 with a Leu to His substitution at position 38, or a sequence having at least 85%, at least 90%, at least 95%, at least 96%, at least 97%, at least 98% or at least 99% to the sequence set forth in SEQ ID NO: 8, provided that the Leu of the DELLA domain corresponding to position 38 of SEQ ID NO: 8 is substituted with a larger basic residue e.g., His. In preferred embodiments, the variant GAI1 protein comprises a sequence which is at least 99.1%, at least 99.2%, at least 99.3%, at least 99.4%, at least 99.5%, at least 99.6%, at least 99.7%, or at least 99.8% identical to the sequence set forth in SEQ ID NO: 8, provided that the Leu of the DELLA domain corresponding to position 38 of SEQ ID NO: 8 is substituted with a larger basic residue e.g., His. In each of the foregoing examples describing a variant GAI1 protein comprising a sequence which has a percentage level of identity relative to the sequence set forth in SEQ ID NO: 8, the variant GAI1 protein may be an orthologue of the sequence set forth in SEQ ID NO: 8 comprising a substitution of the Leu at the position corresponding to residue 38 of SEQ ID NO: 8 e.g., substitution with a larger basic residue, such as a His. In one particular example, the variant GAI1 protein comprises the sequence set forth in SEQ ID NO: 9.

In one example, the variant of the GAI1 gene or fragment thereof which confers dwarf stature is present in a homozygous (GAI1/GAI1) state.

In one example, the variant of the GAI1 gene or fragment thereof which confers dwarf stature is present in a heterozygous (GAI1/Gai1) state.

In one example, the DELLA domain is altered, truncated or deleted from the GAI1 gene or fragment thereof e.g., as a result of the one or more mutations.

In another example, the GAI1 protein or the DELLA domain thereof is silenced e.g., post-transcriptionally silenced. In accordance with this example, the polynucleotide which confers a dwarf stature to the plant may be an RNAi agent targeting a mRNA transcript of the GAI1 protein e.g., such as corresponding to the DELLA domain.

In each of the foregoing examples, the plant or part thereof produces parthenocarpic seedless fruit when flowers are unpollinated and fruit containing seeds when flowers are pollinated with viable pollen.

In each of the foregoing examples, the plant or part thereof further comprises a polynucleotide that confers stenospermocarpy to the plant or part thereof.

In one example, the polynucleotide that confers stenospermocarpy to the plant or part thereof is altered relative to the corresponding wildtype gene or wildtype allele thereof. For example, the polynucleotide that confers stenospermocarpy to the plant or part thereof may comprise one or more mutations relative to the corresponding wildtype gene or wildtype allele thereof. The one or more mutations may be selected from amino acid substitutions, deletions or additions.

In one example, the polynucleotide that confers stenospermocarpy to the plant or plant part is a variant of the Vitis vinifera MADS-box protein 5 (VvMADS5) locus.

In one example, the VvMADS5 locus encodes a VvMADS5 protein comprising the amino acid sequence set forth in SEQ ID NO: 10, and the variant VvMADS5 protein comprises a substitution of the Arg at position 197 of the sequence set forth in SEQ ID NO: 10 with a hydrophobic amino acid e.g., Leu (R197L). In one example, the variant VvMADS5 locus encodes a variant VvMADS5 protein comprising an amino acid sequence set forth in SEQ ID NO: 11, or a sequence having at least 85%, at least 90%, at least 95%, at least 96%, at least 97%, at least 98%, at least 99% identical to the sequence set forth in SEQ ID NO: 11 provided that the amino acid at position 197 relative to SEQ ID NO: 11 is a hydrophobic amino acid e.g., Leu. For example, the variant VvMADS5 locus may encode a variant VvMADS5 protein comprising an amino acid sequence which is at least 99.1%, at least 99.2%, at least 99.3%, at least 99.4%, or at least 99.5% identical to the sequence set forth in SEQ ID NO: 11 provided that the amino acid at position 197 relative to SEQ ID NO: 11 is a hydrophobic amino acid e.g., Leu. In each of the foregoing examples describing a variant VvMADS5 locus encoding a variant VvMADS5 protein comprising a sequence which has a percentage level of identity relative to the sequence set forth in SEQ ID NO: 11, the variant VvMADS5 protein may be an orthologue of the sequence set forth in SEQ ID NO: 11 comprising a corresponding amino acid substitution at position 197.

In one example, the variant VvMADS5 locus encodes a variant VvMADS5 protein comprising the amino acid sequence set forth in SEQ ID NO: 11.

In one example, the variant VvMADS5 locus which confers stenospermocarpy is present in a homozygous state.

In one example, the variant VvMADS5 locus which confers stenospermocarpy is present in a heterozygous state.

In one example, the variant VvMADS5 locus comprises one or more mutations which results in deletion or truncation of the VvMADS5 protein.

In another example, the VvMADS5 protein is silenced e.g., post-transcriptionally silenced. In accordance with this example, the polynucleotide which confers stenospermocarpy to the plant may be an RNAi agent targeting a mRNA transcript encoded by the VvMADS5 locus.

The present disclosure also provides a plant or part thereof which produces seedless fruit, said plant comprising:
(i) a flower sex (FSL) locus genotype which is heterozygous (FSL/fsl) as described herein, or homozygous for the hermaphrodite FSL locus allele (FSL/FSL) as described herein;
(ii) a polynucleotide that confers dwarf stature to a plant as described herein; and
(iii) polynucleotide that confers stenospermocarpy as described herein.

In each of the foregoing examples describing a plant or plant part which further comprises a polynucleotide that confers stenospermocarpy, the plant produces parthenocarpic seedless fruit when flowers are unpollinated and stenospermocarpic fruit when flowers are pollinated with viable pollen.

In one example, the plant as described herein is a dioecious plant species.

In another example, the plant as described herein is a hermaphroditic plant species.

In each of the foregoing examples, the plant is preferably a fruit producing plant i.e., an angiosperm. For example, the plant may be a berry producing plant, a hesperidia producing plant, a drupe producing plant, a pome producing plant, or a pepo producing plant.

In one example, the plant is a berry producing plant. For example, the plant may be a Vitis sp. e.g., a Vitis species selected from the group consisting of: Vitis vinifera, Vitis, lambrusca, Vitis rotundifolia, Vitis aestivalis and hybrids thereof. In one example, the Vitis sp produces table grapes. In another example, the Vitis sp produces wine grapes.

In one example, the plant part is a cell, seed, a fruit, a root, a plant cutting or scion.

Also provided herein is a method of controlling flower sex in a plant, said method comprising altering a level of FSL polypeptide activity in the plant or part thereof compared to a level of FSL polypeptide activity in a corresponding plant or part thereof having a FSL locus genotype which confers a male or hermaphrodite flower phenotype. In one example, the plant or part thereof has an altered level of FSL polypeptide activity compared to a corresponding plant or part thereof which expresses a FSL polypeptide encoded by a wildtype hermaphrodite allele of the FSL locus. In another example, the plant or part thereof has an altered level of FSL polypeptide activity compared to a corresponding plant or part thereof which expresses a FSL polypeptide encoded by a wildtype male allele of the FSL locus. Exemplary FSL polypeptides encoded by wildtype hermaphroditic and male alleles of the FSL locus are described herein.

In one example, an FSL locus genotype which confers a hermaphrodite flower phenotype comprises a hermaphrodite allele of the FSL locus encoding the FSL polypeptide comprising an amino acid sequence set forth in SEQ ID NO:1, a biologically active fragment thereof, or an amino acid sequence which is at least 40% identical to the sequence set forth in SEQ ID NO:1; and

an FSL locus genotype which confers a male flower phenotype comprises a male allele of the FSL locus encoding the FSL polypeptide comprising an amino acid sequence set forth in SEQ ID NO:3, a biologically active fragment thereof, or an amino acid sequence which is at least 40% identical to the sequence set forth in SEQ ID NO:3.

In some examples, a plant or plant part having an altered level of FSL polypeptide activity comprises an FSL polypeptide comprising an amino acid sequence set forth in SEQ ID NO:2, a biologically active fragment thereof, or an amino acid sequence which is at least 40%, at least 50%, at least 60%, at least 70%, at least 80%, at least 85%, at least 90%, at least 95%, at least 96%, at least 97%, at least 98%, or at least 99% identical to the sequence set forth in SEQ ID NO:2.

In one example the method comprises modifying a FSL locus comprising a polynucleotide sequence encoding the FSL polypeptide or biologically active fragment thereof. For example, the method may comprise modifying a region of the FSL locus encoding a plant AT-rich sequence- and zinc-binding (PLATZ) domain e.g., relative to a corresponding polynucleotide sequence of a wildtype hermaphroditic or male allele of the FSL locus encoding a PLATZ domain. Modifying a region of the FSL locus may comprise introducing one or more nucleotide additions, deletions or substitutions to the polynucleotide sequence encoding the FSL polypeptide relative to the corresponding polynucleotide sequence of a wildtype FSL locus allele which confers a male or hermaphrodite flower phenotype when expressed. For example, the polynucleotide sequence encoding the FSL polypeptide may be modified relative to a corresponding polynucleotide sequence of a wildtype hermaphrodite allele of the FSL locus. For example, the polynucleotide sequence encoding the FSL polypeptide may be modified relative to a corresponding polynucleotide sequence of a wildtype male allele of the FSL locus. In one example, the polynucleotide sequence encoding the FSL polypeptide may have an ORF which comprises one or more (e.g., 1, 2, 3, 4, 5, 6, 7, 8, 9, 10 or more) nucleotide additions, deletions or substitutions between positions 153 and 189, such as between positions 155 and 159, relative to the sequence set forth in SEQ ID NO: 6 or 7 (or at one or more corresponding nucleotide positions of the corresponding genomic sequence). For example, the polynucleotide sequence encoding the FSL polypeptide may have an ORF which comprises one or more (e.g., 1, 2, 3, 4, 5, 6, 7, 8, 9, 10 or more) nucleotides deleted between positions 153 and 189 relative to the sequence set forth in SEQ ID NO: 6 or 7 (or at one or more corresponding nucleotide positions of the corresponding genomic sequence). For example, the polynucleotide sequence encoding the FSL polypeptide may have an ORF which comprises one or more T's (e.g., T, TT or TTT) deleted between positions 155 and 159 relative to the sequence set forth in SEQ ID NO: 6 or 7 (or one or more T's deleted at one or more corresponding nucleotide positions of the corresponding genomic sequence). For example, the polynucleotide sequence encoding the FSL polypeptide may have an ORF which comprises one or more T's (e.g., T, TT or TTT) added between positions 155 and 159 relative to the sequence set forth in SEQ ID NO: 6 or 7 (or one or more T's added at one or more corresponding nucleotide positions of the corresponding genomic sequence).

In one example, modifying the FSL locus is achieved using a gene-editing technology. For example, a polynucleotide sequence encoding the FSL polypeptide may be gene-edited using CRISPR, TALON or ZFN technology, or a combination thereof.

In one example, the FSL polypeptide or biologically active fragment thereof encoded by the modified polynucleotide sequence comprises one or more amino acid additions, deletions or substitutions relative to the FSL polypeptide encoded by the corresponding wildtype FSL locus allele (e.g., as a result of one or more nucleotide additions, deletions or substitutions to the encoding polynucleotide sequence). For example, modifying a polynucleotide at the FSL locus may result in one or more amino acid additions, deletions or substitutions in the PLATZ domain of the FSL polypeptide relative to a the corresponding wildtype amino acid sequence. In some examples, the FSL polypeptide encoded by the modified polynucleotide sequence is truncated. In some examples, the FSL polypeptide or a domain thereof e.g., the PLATZ domain, encoded by the modified polynucleotide sequence is absent from the plant or part thereof.

In further examples, the level of FSL polypeptide activity in the plant or part thereof is altered by post-transcriptional silencing with an RNA interference (RNAi) agent which targets a messenger RNA (mRNA) of the FSL locus. In accordance with this example, the method may comprise introducing to the plant or part thereof a RNAi agent which targets a mRNA of the FSL locus or an allele thereof. For example, the plant or part thereof may be transfected with and/or have incorporated into its genome a construct for expressing the RNAi agent e.g., an expression vector which expresses the RNAi agent. The RNAi agent may be any RNAi agent known in the art or described herein.

In some examples, the polynucleotide sequence of the FSL locus is modified relative to the polynucleotide sequence of a corresponding wildtype FSL locus allele.

In one example, the corresponding wildtype FSL locus allele may be a hermaphroditic allele of the FSL locus. In accordance with this example, the ORF of the corresponding wildtype FSL locus allele may comprises a polynucleotide sequence set forth in SEQ ID NO: 6, a sequence which is at least 60% identical thereto, or an orthologous sequence thereof corresponding to the species of plant. In one example, the ORF of the corresponding wildtype FSL locus allele comprises a sequence which is at least 65%, at least 70%, at least 75%, at least 80%, at least 85%, at least 90%, at least 95%, at least 96%, at least 97%, at least 98%, or at least 99% identical to the sequence set forth in SEQ ID NO: 6. In one particular example, the ORF of the corresponding wildtype FSL locus allele comprises a sequence set forth in SEQ ID NO: 6.

In another example, the corresponding wildtype FSL locus allele may be a male allele of the FSL locus. In accordance with this example, the ORF of the corresponding wildtype FSL locus allele may comprises a polynucleotide sequence set forth in SEQ ID NO: 7, a sequence which is at least 60% identical thereto, or an orthologous sequence thereof corresponding to the species of plant. In one example, the ORF of the corresponding wildtype FSL locus allele comprises a sequence which is at least 65%, at least 70%, at least 75%, at least 80%, at least 85%, at least 90%, at least 95%, at least 96%, at least 97%, at least 98%, or at least 99% identical to the sequence set forth in SEQ ID NO: 7. In one particular example, the ORF of the corresponding wildtype FSL locus allele comprises a sequence set forth in SEQ ID NO: 7.

In accordance with examples of the method of controlling flower sex in a plant as described herein, FSL polypeptide activity is reduced in the plant or plant part relative a level of FSL polypeptide activity in a corresponding wildtype plant or part thereof. For example, FSL polypeptide activity in the plant or plant part may be reduced by at least 10%, at least 20%, at least 30%, at least 40%, at least 50%, at least 60%, at least 70%, at least 80%, at least 90%, or at least 95% relative to a level of FSL polypeptide activity in a corresponding wildtype plant or part thereof.

In each of the foregoing examples describing a plant or plant part having a reduced level of FSL polypeptide activity following performance of the method, the reduced FSL polypeptide activity may be caused by a corresponding reduction in expression of FSL polypeptide or reduced activity of the FSL polypeptide or reduced activity of the polynucleotide encoding the FSL polypeptide. Alternatively or in addition, the reduced FSL polypeptide activity may be caused by a corresponding reduction in expression of FSL locus mRNA relative to a level of expression in a corresponding wildtype plant or part thereof e.g., a corresponding hermaphrodite or male wildtype plant or part thereof.

In some examples, FSL polypeptide activity is abrogated in the plant or plant part following performance of the method. For example, FSL polypeptide expression may be completely inhibited or the FSL locus encoding the FSL polypeptide may be knock-out in the plant or part thereof.

In one example, altering the activity of the FSL polypeptide in the plant or part thereof causes a male reproductive part of a flower of the plant to be absent or non-functional. For example, reducing the activity of FSL polypeptide as described herein may cause a male reproductive part of a flower of the plant or plant part to be absent or non-functional. In some examples, reducing the activity of FSL polypeptide as described herein may cause the male reproductive part of a flower to be non-functional (even if it is present). A non-functional male reproductive part of a flower may be underdeveloped due to the altered e.g., reduced, activity of FSL polypeptide, causing it to be non-functional. Accordingly, altering the level of FSL polypeptide in a plant or part thereof may result in a plant or plant part which produces flowers which are phenotypically female or male sterile.

In some examples, the plant or plant part in which FSL polypeptide activity is altered comprises a polynucleotide which confers dwarf stature as described herein. In some example, the plant or plant part in which FSL polypeptide activity is altered already comprises a polynucleotide which confers dwarf stature. In other examples, the method comprises introducing to the plant or plant part the polynucleotide which confers dwarf stature.

In some examples, the plant or plant part in which FSL polypeptide activity is altered comprises a polynucleotide which confers stenospermocarpy as described herein. In some examples, the plant or plant part in which FSL polypeptide activity is altered already comprises a polynucleotide which confers stenospermocarpy. In other examples, the method comprises introducing to the plant or plant part the polynucleotide which confers stenospermocarpy.

In one example, the plant or plant part in which FSL polypeptide activity is altered is a dioecious plant species.

In another example, the plant or plant part in which FSL polypeptide activity is altered is a hermaphroditic plant species.

In each of the foregoing example, the plant in which FSL polypeptide activity is altered is preferably a fruit producing plant i.e., an angiosperm. For example, the plant may be a berry producing plant, a hesperidia producing plant, a drupe producing plant, a pome producing plant, or a pepo producing plant.

In one example, the plant or plant part in which FSL polypeptide activity is altered produces berries. For example, the plant may be a Vitis sp. e.g., a Vitis species selected from the group consisting of: Vitis vinifera, Vitis, lambrusca, Vitis rotundifolia, Vitis aestivalis and hybrids thereof. In one example, the Vitis sp produces table grapes. In another example, the Vitis sp produces wine grapes.

In one example, the plant part is a cell, seed or seed part, a fruit, a root, a plant cutting or scion.

Also contemplated herein is a method of controlling flower sex in a plant comprising increasing a level of activity of FSL polypeptide encoded by a male or hermaphrodite allele of the FSL locus in the plant or part thereof, relative to a level of activity of the corresponding FSL polypeptide in a corresponding plant or part thereof having an FSL locus genotype which confers a female flower phenotype.

In one example, a hermaphrodite allele of the FSL locus encodes an FSL polypeptide comprising an amino acid sequence set forth in SEQ ID NO:1, a biologically active fragment thereof, or an amino acid sequence which is at least 40% identical to the sequence set forth in SEQ ID NO:1. Other exemplary FSL polypeptides encoded by a hermaphrodite allele of the FSL locus are described and contemplated herein. In one particular example, the hermaphrodite allele of the FSL locus encodes the FSL polypeptide comprising the amino acid sequence set forth in SEQ ID NO:1, or a biologically active fragment thereof.

In one example, a male allele of the FSL locus encodes an FSL polypeptide comprising an amino acid sequence set forth in SEQ ID NO:3, a biologically active fragment thereof, or an amino acid sequence which is at least 40% identical to the sequence set forth in SEQ ID NO:3. Other exemplary FSL polypeptides encoded by a male allele of the FSL locus are described and contemplated herein. In one particular example, the male allele of the FSL locus encodes the FSL polypeptide comprising the amino acid sequence set forth in SEQ ID NO:3, or a biologically active fragment thereof.

In one example, increasing the level of activity of FSL polypeptide encoded by a male or hermaphrodite allele of the FSL locus in the plant or part thereof confers a flower phenotype in which functional male reproductive parts are present.

The present disclosure also provides a method of producing a plant that produces flowers of known sex, said method comprising the steps of:

i) crossing two parental plants,

ii) screening one or more progeny plants from the cross to determine the genotype at a flower sex (FSL) locus, and

iii) selecting a progeny plant capable of exhibiting a desired flower sex phenotype on the basis of the FSL locus genotype, wherein an FSL locus genotype which is homozygous for a female FSL locus allele (f/f) confers a female flower phenotype, an FSL locus genotype which is heterozygous for a female FSL locus allele and a hermaphrodite FSL locus allele (f/H) confers a hermaphrodite flower phenotype, an FSL locus genotype which is homozygous for a hermaphrodite FSL locus allele (H/H) confers a hermaphrodite flower phenotype, and an FSL locus genotype which is heterozygous for a male FSL locus allele and either a female FSL locus allele (M/f) or a hermaphrodite FSL locus allele (M/H) confers a male flower phenotype, and an FSL locus genotype which is homozygous for a male FSL locus allele (M/M) confers a male flower phenotype,

thereby producing a plant which produces flowers of known sex.

FSL locus sequences, including male, female and hermaphrodite FSL locus allele sequences, are described herein and shall be taken to apply mutatis mutandis to each and every example describing a method of producing a plant that produces flowers of known sex described herein unless stated otherwise.

In one example, the method comprises selecting a progeny plant having an FSL locus genotype which is homozygous for a female FSL locus allele (f/f) to thereby produce a plant which produces female flowers.

In one example, the method comprises selecting a progeny plant having an FSL locus genotype which is heterozygous for a female FSL locus allele and a hermaphrodite FSL locus allele (f/H) or homozygous for a hermaphrodite FSL locus allele (H/H) to thereby produce a plant which produces hermaphroditic flowers.

In one example, the female allele of the FSL locus has an ORF which comprises a sequence set forth in SEQ ID NO: 5, or a sequence having at least 70% identity thereto provided that the nucleotide corresponding to position 621 of the sequence set forth in SEQ ID NO: 5 is a A. For example, the female allele of the FSL locus may comprise an ORF comprising a sequence having at least 75%, at least 80%, at least 85%, at least 90%, at least 95%, at least 96%, at least 97%, at least 98% or at least 99% identity to the sequence set forth in SEQ ID NO: 5 provided that the nucleotide corresponding to position 621 of the sequence set forth in SEQ ID NO: 5 is a A. In some examples, the ORF of the female allele of the FSL locus comprises the sequence set forth in SEQ ID NO: 5.

In each of the foregoing examples describing an ORF of a female allele of the FSL locus which has a percentage level of identity to the sequence set forth in SEQ ID NO: 5, the ORF of the FSL locus may be an orthologue of the sequence set forth in SEQ ID NO: 5 corresponding to the plant species.

In one example, the hermaphrodite allele of the FSL locus has an ORF which comprises a sequence set forth in SEQ ID NO: 6, or a sequence having at least 70%, at least 75%, at least 80%, at least 85%, at least 90%, at least 95%, at least 96%, at least 97%, at least 98%, at least 99% identity to the sequence set forth in SEQ ID NO: 6 provided that the nucleotide corresponding to position 627 of the sequence set forth in SEQ ID NO: 6 is a C. In some examples, the ORF of the hermaphrodite allele of the FSL locus comprises the sequence set forth in SEQ ID NO: 6.

In each of the foregoing examples describing an ORF of a hermaphrodite allele of the FSL locus which has a percentage level of identity to the sequence set forth in SEQ ID NO: 6, the ORF of the FSL locus may be an orthologue of the sequence set forth in SEQ ID NO: 6 corresponding to the plant species.

The present disclosure also provides a method of producing a plant which produces seedless fruit, said method comprising the steps of:

i) crossing two parental plants, wherein one of the parental plants comprises an FSL locus which is homozygous for a female allele (f/f) conferring female flower phenotype, and the other parental plant comprises a polynucleotide that confers dwarf stature,

ii) screening one or more progeny plants from the cross for the presence or absence of the FSL locus which is homozygous for a female allele (f/f), and the presence or absence of the polynucleotide that confers dwarf stature, and

iii) selecting a progeny plant which comprises the FSL locus which is homozygous for a female allele (f/f) and which comprises the polynucleotide that confers dwarf stature, thereby producing a plant which produces seedless fruit.

The present disclosure also provides a method of producing a plant which produces seedless fruit, said method comprising the steps of:

i) crossing two parental plants, wherein at least one of the parental plants comprises (a) a flower sex (FSL) locus which is homozygous fsl/fsl conferring a female flower phenotype, homozygous (FSL/FSL), or heterozygous or homozygous (FSL/fsl) conferring a hermaphrodite flower phenotype, (b) at least one of the parental plants comprises a polynucleotide that confers dwarf stature, and (c) at least one of the parental plants comprises a polynucleotide that confers stenospermocarpy,

ii) screening one or more progeny plants from the cross for the presence or absence of the FSL locus which is homozygous for a female phenotype (fsl/fsl), homozygous in the hermaphrodite phenotype (FSL/FSL), or heterozygous for a hermaphrodite phenotype (FSL/fsl), (b) the presence or absence of the polynucleotide that confers dwarf stature, and (c) the presence or absence of the polynucleotide that confers stenospermocarpy, and

iii) selecting a progeny plant which comprises (a) an FSL locus genotype that confers a female or hermaphrodite flower phenotype, (b) a polynucleotide that confers dwarf stature, and (c) the polynucleotide that confers stenospermocarpy,

thereby producing a plant which produces seedless fruit.

A progeny plant which comprises (a) an FSL locus genotype that confers a hermaphrodite flower phenotype, (b) the polynucleotide that confers dwarf stature, and (c) the polynucleotide that confers stenospermocarpy, produces stenospermocarpic seedless fruit.

A progeny plant which comprises (a) an FSL locus genotype that confers a female flower phenotype, (b) the polynucleotide that confers dwarf stature, and (c) the polynucleotide that confers stenospermocarpy, produces parthenocarpic seedless fruit.

The present disclosure also provides a method of producing a plant which produces seedless fruit, said method comprising the steps of:

i) crossing two parental plants, wherein at least one of the parental plants comprises an FSL locus which is homozygous for a female allele (f/f) conferring female flower phenotype, at least one of the parental plants comprises a polynucleotide that confers dwarf stature, and at least one of the parental plants comprises a polynucleotide that confers stenospermocarpy,

ii) screening one or more progeny plants from the cross for the presence or absence of the FSL locus which is homozygous for a female allele (f/f), the presence or absence of the polynucleotide that confers dwarf stature, and the presence or absence of the polynucleotide that confers stenospermocarpy, and

iii) selecting a progeny plant which comprises the FSL locus which is homozygous for a female allele (f/f), the polynucleotide that confers dwarf stature, and the polynucleotide that confers stenospermocarpy,

thereby producing a plant which produces parthenocarpic seedless fruit.

The present disclosure also provides a method of producing a plant which produces seedless fruit, said method comprising the steps of:

i) crossing two parental plants, wherein at least one of the parental plants comprises a polynucleotide that confers dwarf stature, and at least one of the parental plants comprises a polynucleotide that confers stenospermocarpy,

ii) screening one or more progeny plants from the cross for the presence or absence of the polynucleotide that confers dwarf stature, and the presence or absence of the polynucleotide that confers stenospermocarpy, and

iii) selecting a progeny plant which comprises the polynucleotide that confers dwarf stature, and the polynucleotide that confers stenospermocarpy,

thereby producing a plant which produces parthenocarpic seedless fruit.

In each of the foregoing examples describing methods of producing seedless fruit, the method may further comprise:

iv) backcrossing the progeny selected at iii) with plants of the same genotype as a one or the parent plants, but lacking the polynucleotide(s) for which the progeny were selected, a sufficient number of times to produce a plant with a majority of the genotype of the parent but comprising the polynucleotide(s) of interest, and

iv) selecting a progeny plant which has the polynucleotides of interest, preferably wherein the progeny comprises a hermaphrodite FSL locus allele or a female FSL locus allele or both, and more preferably wherein the progeny is homozygous for the female FSL locus allele.

The female allele of the FSL locus has previously been described herein and shall be taken to apply mutatis mutandis to each and every example of the method of producing a plant which produces seedless fruit as described herein unless stated otherwise. In one particular example, the female allele of the FSL locus has an ORF which comprises a sequence set forth in SEQ ID NO: 5.

Exemplary polynucleotides which confer dwarf stature and stenospermocarpy, respectively, are described herein and shall apply mutatis mutandis to each and every example of the method of producing a plant which produces seedless fruit as described herein unless stated otherwise. In one particular example, the polynucleotide that confers dwarf stature is a variant of the GAI1 gene encoding a variant GAI1 protein comprising a sequence set forth in SEQ ID NO: 9. In one particular example, the polynucleotide that confers stenospermocarpy is a variant of the VvMADS5 locus that encodes a variant VvMADS5 protein comprising a sequence set forth in SEQ ID NO: 11.

In one example, the plant which produces seedless fruit is a dioecious plant species.

In one example, the plant which produces seedless fruit is a hermaphroditic plant species.

In one example, the plant which produces seedless fruit is a berry producing plant, a hesperidia producing plant, a drupe producing plant, a pome producing plant, or a pepo producing plant.

In one example, the plant produces seedless berries. For example, the plant may be a Vitis sp. e.g., a Vitis species selected from the group consisting of: Vitis vinifera, Vitis, lambrusca, Vitis rotundifolia, Vitis aestivalis and hybrids thereof. In one example, the Vitis sp produces table grapes. In another example, the Vitis sp produces wine grapes.

The present disclosure also provides a plant or part thereof produced by the method described herein.

In one example, the plant part is a cell seed or seed part, a fruit, a root, a plant cutting or scion.

Also provided herein is fruit produced from a plant described herein.

In one particular example, the plant is a Vitis sp. and the fruit are grapes.

In one example, the fruit is seedless. In one example the fruit are stenospermocarpic seedless. In one example the fruit are parthenocarpic seedless.

The present disclosure also provides a method of producing fruit, the method comprising growing a plant as described herein to thereby produce fruit.

In one example, the method of producing fruit further comprises harvesting the fruit produced from the plant.

In one example, the method of producing fruit further comprises processing the fruit.

For example, processing the fruit may comprise packaging the fruit. For example, processing the fruit may comprise producing one or more product from the fruit.

The present disclosure also provides a product produced from a plant as described herein or fruit thereof.

In one example, the product is a food product, food ingredient, beverage product or beverage ingredient. The food product may be selected from the group consisting of table grapes, jam, marmalade, jelly, sultanas, and raisins, for example. The food ingredient may be vincotto, verjuice, vinegar or grape must syrup (mosto cotto), for example. The beverage product may be is wine, grappa, brandy or grape juice, for example. The beverage ingredient may be wine grapes, table grapes or juice therefrom, for example.

In one example, the present disclosure provides a FSL polypeptide as described herein. For example, the FSL polypeptide may comprise an amino acid sequence selected from the group consisting of: a) sequences set forth in SEQ ID NO: 1, 2 or 3, or a biologically active fragment of any one thereof, or b) an amino acid sequence which is at least 40% identical, at least 50%, at least 60%, at least 70%, at least 80%, at least 85%, at least 90%, at least 95% at least 96% at least 97%, at least 98% or at least 99% identical to a sequence set forth in SEQ ID NO: 1, 2 or 3.

In another example, the present disclosure provides an isolated nucleic acid molecule comprising a polynucleotide sequence encoding an FSL polypeptide as described herein. For example, the nucleic acid molecule may comprise a) a polynucleotide sequence set forth in SEQ ID NOs: 4, 5, 6 or 7 or a polynucleotide sequence having an ORF set forth in SEQ ID NOs: 4, 5, 6 or 7, b) a polynucleotide sequence which is at least 40% identical, at least 50%, at least 60%, at least 70%, at least 80%, at least 85%, at least 90%, at least 95% at least 96% at least 97%, at least 98% or at least 99% identical to a sequence set forth in SEQ ID NO: 4, 5, 6 or 7 or a polynucleotide sequence having an ORF which is at least 40% identical, at least 50%, at least 60%, at least 70%, at least 80%, at least 85%, at least 90%, at least 95% at least 96% at least 97%, at least 98% or at least 99% identical to a sequence set forth in SEQ ID NOs: 4, 5, 6 or 7, or c) a polynucleotide sequence which is complementary to a polynucleotide sequence of a) or b).

In one example, the isolated nucleic acid molecule comprises a recombinant polynucleotide.

The present disclosure also provides an expression vector comprising the isolated nucleic acid molecule as described herein.

In one example, the isolated nucleic acid molecule is operably linked to a promoter.

In one example, the expression vector is a plasmid or virus.

The present disclosure also provides an isolated cell of a plant as described herein.

The present disclosure also provides a host cell comprising a nucleic acid molecule as described herein or an expression vector comprising same as described herein. The host cell may be a yeast, bacteria or plant cell.

The present disclosure also provides a method of determining flower sex of a plant, said method comprising performing one or more assays on a sample obtained from the plant to determine the genotype of the plant at a flower sex (FSL) locus and determining flower sex of a plant based on the FSL locus genotype,

wherein a plant which comprises an FSL locus genotype which is homozygous for a female FSL locus allele (f/f) will produce flowers which are phenotypically female,

wherein a plant which comprises an FSL locus genotype which is heterozygous for a female FSL locus allele and a hermaphrodite FSL locus allele (f/H) will produce flowers which are phenotypically hermaphroditic,

wherein a plant which comprises an FSL locus genotype which is homozygous for a hermaphrodite FSL locus allele (H/H) will produce flowers which are phenotypically hermaphroditic, and

wherein a plant which comprises an FSL locus genotype which is heterozygous for a female FSL locus allele and a male FSL locus allele (f/M) will produce flowers which are phenotypically male.

In one example the female allele of the FSL locus has an ORF which comprises a sequence set forth in SEQ ID NO: 5, or a sequence having at least 70% identity thereto provided that the nucleotide corresponding to position 621 of the sequence set forth in SEQ ID NO: 5 is a A. Exemplary female FSL locus allele sequences are described herein. In some examples, the female allele of the FSL locus has an ORF which comprises the sequence set forth in SEQ ID NO: 5.

In each of the foregoing examples describing a female allele of the FSL locus having an ORF which has a percentage level of identity to the sequence set forth in SEQ ID NO: 5, the female allele of the FSL locus may be an orthologue of the sequence set forth in SEQ ID NO: 5 corresponding to the plant species.

In one example, the hermaphrodite allele of the FSL locus has a ORF which comprises a sequence set forth in SEQ ID NO: 6, or a sequence having at least 70% identity thereto provided that the nucleotide corresponding to position 627 of the sequence set forth in SEQ ID NO: 6 is a C. Exemplary hermaphrodite FSL locus allele sequences are described herein.

In some examples, the hermaphrodite allele of the FSL locus has an ORF which comprises the sequence set forth in SEQ ID NO: 6.

In each of the foregoing examples describing a hermaphrodite allele of the FSL locus having an ORF which has a percentage level of identity to the sequence set forth in SEQ ID NO: 6, the hermaphrodite allele of the FSL locus may be an orthologue of the sequence set forth in SEQ ID NO: 6 corresponding to the plant species.

In one example, the male allele of the FSL locus has an ORF which comprises a sequence set forth in SEQ ID NO: 7, or a sequence having at least 70% identity thereto provided that the nucleotide corresponding to position 627 of the sequence set forth in SEQ ID NO:7 is an C. Exemplary male FSL locus allele sequences are described herein. In some examples, the male allele of the FSL locus has an ORF which comprises the sequence set forth in SEQ ID NO: 7.

In each of the foregoing examples describing a male allele of the FSL locus has an ORF which has a percentage level of identity to the sequence set forth in SEQ ID NO: 7, the male allele of the FSL locus may be an orthologue of the sequence set forth in SEQ ID NO: 7 corresponding to the plant species.

In one example, the genotype of the plant at the FSL locus is determined by a PCR-based assay.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. (a) DNA sequence for the FSL hermaphrodite allele (H) from Cabernet sauvignon;

FIG. 1. (b) DNA sequence for the FSL female allele (f) from Vitis sp. clone 04C023V0003;

FIG. 1. (c) DNA sequence for the FSL hermaphrodite allele (H) from Vitis sp. clone 04C023V0006. FIG. 1. (d) DNA sequence for the FSL male allele (M) from Vitis sp. clone 04C023V0016. In each of FIG. 1(a)-FIG. 1(d), the open reading frame is underlined and the sequence encoding the PLATZ domain is bolded.

FIG. 2. Multiple sequence alignment of open reading frames (ORFs) for the female allele (SEQ ID NO:5), hermaphrodite allele (SEQ ID NO:6), and male allele (SEQ ID NO:7) of the FSL locus performed using CLUSTAL O (1.2.4).

FIG. 3. (a) Protein sequence for the FSL hermaphrodite allele (H) from Cabernet sauvignon and Vitis sp. clone 04C023V0006; FIG. 3. (b) Protein A sequence for the FSL female allele (f) from Vitis sp. clone 04C023V0003; FIG. 3. (c) Protein sequence for the FSL hermaphrodite allele (H) from Vitis sp. clone 04C023V0006. In each of FIG. 3(a)-FIG. 3(c), the sequence encoding the PLATZ domain is bolded.

FIG. 4. A phylogenetic tree for the hermaphrodite protein sequence from Vitus vinifera.

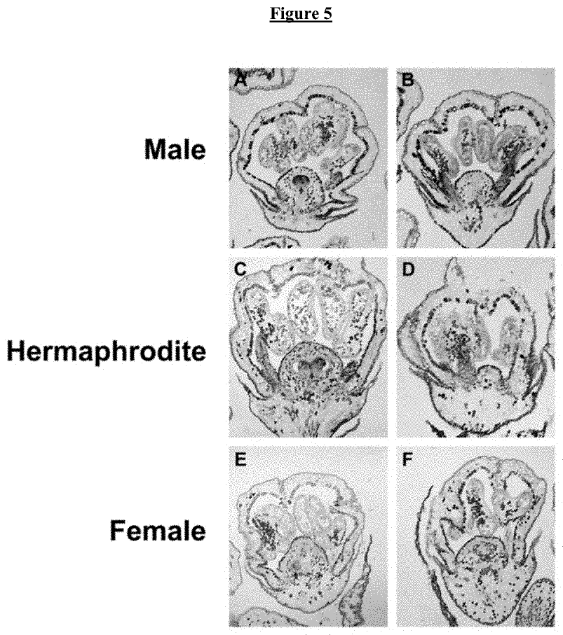

FIG. 5. Shows expression of FSL at stage 1-2 of flower development as determined by the modified E-L system (Coombe (1995)). In situ hybridization was used to localize FSL transcripts in (A and B) male, (C and D) hermaphrodite and (E and F) female flowers. The perianth (p) organs encapsulate the reproductive organs. The red and green arrows point at the anthers and filaments of the stamens in (A). The ovule is marked by the blue arrow.

FIG. 6. Relative gene expression of FSL in leaves and early flowers as measured by RT-qPCR

FIG. 7. In vitro screening of CRISPR guide RNAs targeting the FSL locus in Vitis vinifera. Guide RNAs designated sgRNAFS1 and sgRNAFS4 were selected for CRISPR editing of microvines.

FIG. 8. Genetic transformation of the microvines with the CRISPR constructs targeting FSL.

FIG. 9. Illustrates differences in flower phenotype between the hermaphrodite and the FSL gene edited plant. The hermaphrodite flower has erect stamens (A) whereas the FSL gene edited plant has poorly formed stamens with retracted filaments (B). Pollen is viable in hermaphrodite flowers as seen by the extension of the pollen tube (C) whereas the FSL edited gene has no fertile pollen (D) as determined by the pollen germination assay.

FIG. 10. Shows the highest frequency mutations found in FSL knock out plant. The top 13 mutation frequencies show that the majority of mutations is either a T insertion or T gelation at the 16th base of the guide RNA. Note—only 17 bp of the total 20 bp guide is shown.

FIG. 11. Amino acid alignment of FSL knock out and H allele. Both T insertion and T deletion give rise to a nonsense mutation which results in early termination of protein synthesis.

FIG. 12. Shows parthenocarpic seedless fruit from a female microvine without pollination (A) and when pollinated with pollen to produce viable brown seeds (B).

FIG. 13. Shows parthenocarpic and stenospermocarpic seedless fruit from a female microvine (A) and when pollinated with pollen to produce non-viable seed traces (B).

FIG. 14. Shows stenospermocarpic seedless fruit from a hermaphrodite microvine (A). A typical hermaphrodite microvine with brown seeds (B).

FIG. 15. Provides a schematic of the CRISPR/Cas9 vector and the cloning position for the guide RNAs designated FS1 and FS4.

FIG. 16. Provides a DNA sequence alignment for mutants of the FSL locus achieved for both FS1 and FS4, showing the types and locations of mutations that occurred at a frequency of >10%. The mutations for both FS1 and FS4 mostly involved the base T and occurred 5 prime of the PAM site.

FIG. 17. Genomic DNA sequence alignment for homozygous mutants of the FSL locus obtained in the T1 generation CRISPR/Cas9 flower sex lines. The DNA sequences were translated and aligned for the hermaphrodite locus and the homozygous mutated lines. Four lines mutated with the FS4 guide and 3 lines mutated with the FS1 guide. FS1 guide sequence resulted in a T insertion or a T deletion or a double deletion at position 157 bp from the start codon. The FS4 guide sequence resulted in a 5 bp deletion, a T insertion at position 184 bp from the start codon and a CT deletion 182 bp from the start codon and a 10 bp deletion 180 bp from the start codon. Mutations are underlined. The exon intron boundary is shown with an arrow.

FIG. 18. An amino acid sequence alignment for homozygous mutants of the FSL locus obtained in the T1 generation CRISPR/Cas9 flower sex lines obtained for both FS1 and FS4 guide RNAs. The alignment shows the affect of the mutations on the protein sequence.

### KEY TO THE SEQUENCE LISTING

SEQ ID NO: 1 is an amino acid sequence corresponding to the FSL hermaphrodite allele (H) from Vitis vinifera.

SEQ ID NO: 2 is an amino acid sequence corresponding to the FSL female allele (f) from a variety of Vitis vinifera.

SEQ ID NO: 3 is an amino acid sequence corresponding to the FSL male allele (M) from a variety of Vitis vinifera.

SEQ ID NO: 4 is an open reading frame (ORF) DNA sequence corresponding to the FSL hermaphrodite allele (H) from Cabernet sauvignon.

SEQ ID NO: 5 is an open reading frame (ORF) sequence corresponding to the FSL female allele (f) from a variety of Vitis vinifera.

SEQ ID NO: 6 is an open reading frame (ORF) sequence corresponding to the FSL hermaphrodite allele (H) from a variety of Vitis vinifera.

SEQ ID NO: 7 is an open reading frame (ORF) sequence corresponding to the FSL male allele (M) from a variety of Vitis vinifera.

SEQ ID NO: 8 is an amino acid sequence of the Gibberellic Acid Insensitive (GAI1) DELLA protein encoded by the GAI1 gene in Vitis vinifera.

SEQ ID NO: 9 is an amino acid sequence of the variant GAI1 protein comprising Leu to His substitution which is encoded by the variant GAI1 gene in Vitis vinifera.

SEQ ID NO: 10 is an amino acid sequence of the Vitus vinifera MADS-box 5 (VvMADS5) protein encoded by the VvMADS5 gene in Vitis vinifera.

SEQ ID NO: 11 is an amino acid sequence of the variant VvMADS5 protein encoded by the variant VvMADS5 gene in Vitis vinifera.

SEQ ID NO: 12 is a DNA sequence for a primer designated oligo dT B26.

SEQ ID NO: 13 is a DNA sequence for a primer designated CSFS1_CDS_F1.

SEQ ID NO: 14 is a DNA sequence for a primer designated FSL_RT_F1.

SEQ ID NO: 15 is a DNA sequence for a primer designated FSL_RT_R1.

SEQ ID NO: 16 is a DNA sequence corresponding to the single guide RNA (sgRNA) designated “Guide FS1” (in antisense orientation).

SEQ ID NO: 17 is a DNA sequence corresponding to the single guide RNA (sgRNA) designated “Guide FS4” (in antisense orientation).

SEQ ID NO: 18 is a DNA sequence corresponding to the single guide RNA (sgRNA) designated “Guide FS2” (in antisense orientation).

SEQ ID NO: 19 is a DNA sequence corresponding to the single guide RNA (sgRNA) designated “Guide FS3” (in sense orientation).

SEQ ID NO: 20 is a DNA sequence for a primer designated VvSDLF1.

SEQ ID NO: 21 is a DNA sequence for a primer designated VvSDLF2.

SEQ ID NO: 22 is a DNA sequence for a primer designated VvSDLRev.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (for example, plant molecular genetics, plant breeding, cell culture, protein chemistry, wine production and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

### Flower Sex

As used herein, the term “flower” refers to the reproductive structure of a flowering plant (an angiosperm). Flowers are generally formed of two parts: the vegetative part, consisting of petals and associated structures in the perianth, and the reproductive or sexual parts. A “flower” may possess both the male and female reproductive parts (in which the flower may be hermaphroditic), or a may possess male or female reproductive parts only, in which case a flower may be a male flower or a female flower, respectively. The male reproductive part is generally referred to as the “stamen” and the female reproductive part is referred to as the “pistil”. The stamen has two parts: anthers and filaments. The anthers carry the pollen and are generally held up by a thread-like part called a filament. The pistil has three parts: stigma, style, and ovary. The stigma is a sticky structure at the top of the pistil which traps and holds pollen which is transferred from the anthers. The style is the tube-like structure that holds up the stigma and which leads down to the ovary that contains the ovules (or eggs). Depending on whether a flower is a male flower, a female flower or a hermaphroditic flower, it will have male reproductive parts only, female reproductive parts only, or both female and male reproductive parts, respectively.

It will be understood to those of skill in the art that plants can be monoecious, dioecious or hermaphroditic. A “monoecious plant” shall be understood to mean a plant having both the male and female reproductive systems on the same plant i.e., a plant that possess some flowers that are female and others that are male. A “male flower” is a flower which develops a pollen-laden stamen in the absence of a developed pistil, whereas a “female flower” is a flower which develop ovule-holding pistil in the absence of a developed stamen. A “dioecious plant”, on the other hand, shall be understood to mean a plant in which the male and female reproductive systems occur on separate plants. That is, one plant has the male reproductive parts (flowers with pollen-laden stamen) and the other plant has the female parts (flowers with ovule-holding pistil). Flowers which are either male or female (as is the case for dioecious and monoecious plants) are also sometimes termed “imperfect flowers”. A “hermaphroditic plant” or “hermaphrodite” shall be understood to mean a plant that produces flowers containing both male and female reproductive parts (i.e., pollen-laden stamen and ovule-holding pistil). Hermaphroditic plants are largely self-pollinating and truly bisexual. Flowers from hermaphroditic plants are also sometimes termed “perfect flowers”.

As used herein, the term “female flower phenotype”, “phenotypically female flower” or similar shall be understood to mean a flower which has functional female reproductive parts only and exhibits a female flower phenotype. In some examples, a flower which exhibits a female flower phenotype may be a genetically hermaphroditic flower in which the male reproductive parts are non-functional and/or absent i.e., due to reduced or absent FSL polypeptide activity in the plant. In accordance with this example, the reduced or absent FSL polypeptide activity in the plant prevents or inhibits development and/or maturation of the male reproductive part of the flower. A “genetically hermaphroditic flower” will be understood to mean a flower having a hermaphroditic genotype i.e., HH=hermaphrodite or Hf=hermaphrodite, at the FSL locus. Similarly, a “hermaphrodite flower phenotype”, “hermaphroditic flower phenotype” or similar, shall be understood to a flower which has functional male and female reproductive parts. It follows then that the term “male flower phenotype” is intended to refer to a flower which has functional male reproductive parts only.

As used herein, the term “controlling flower sex in a plant” or similar shall be understood to mean controlling or influencing whether a plant develops flowers that are phenotypically male, female or hermaphroditic. That is, controlling whether a plant will develop flowers with male reproductive parts (pollen-laden stamen) only, female reproductive parts (ovule-holding pistil) only, or both.

The term “flower sex (FSL) locus” or “FSL locus” or “FSL gene” as used herein shall be understood to mean a gene or locus that encodes a polypeptide (referred to herein as a FSL polypeptide) which the inventors have shown to be responsible for flower sex or flower gender in angiosperms. The inventors have characterised female (f) and hermaphroditic (H) and male (M) alleles of the FSL locus in Vitis vinifera, the open reading frames (ORF) DNA sequences of which are set forth in SEQ ID NOs: 5-7 respectively. Reference herein to an “FSL locus” is therefore intended to encompass ORFs of the FSL locus allele sequences set forth in SEQ ID NOs: 5-7, as well as FSL locus sequences having at least 60% identity thereto (e.g., having at least 65%, or at least 70%, or at least 75%, or at least 80%, or at least 85%, or at least 90%, or at least 95%, or at least 96%, or at least 97%, or at least 98% or at least 99% identity to the sequence set forth in SEQ ID NOs: 5-7). Also contemplated are orthologues of those sequences which correspond to the particular plant species of interest.

The term “locus” (loci plural) shall be understood to mean a specific place or location on a chromosome where a gene, polynucleotide or genetic marker is found.

As used herein, the term “allele(s)” means any one or more alternatives forms or variants of a gene or polynucleotide sequence at a particular locus, all of which relate to a common trait or characteristic. In a polyploid (e.g., diploid) cell of a plant or plant part, one allele is present on each chromosome of a pair of homologous chromosomes at corresponding positions. In the context of the FSL locus, the term “allele” is used herein to define alternative forms of the FSL locus which the inventors have shown to be associated with different flower sex phenotypes. For example, the inventors have characterised female (f) and hermaphroditic (H) and male (M) alleles of the FSL locus in Vitis vinifera, the ORF DNA sequences of which are set forth in SEQ ID NOs: 5-7 respectively. Accordingly, reference herein to a “female allele of the FSL locus”, a “female FSL locus allele” or similar shall be understood to refer to a variant of the FSL locus which is associated with a female flower phenotype. Similarly, reference herein to a “hermaphrodite allele of the FSL locus”, “hermaphroditic allele of the FSL locus”, “hermaphrodite FSL locus allele”, “hermaphroditic FSL locus allele” or similar shall be understood to refer to a variant of the FSL locus which is associated with a hermaphrodite flower phenotype. Reference herein to a “male allele of the FSL locus”, a “male FSL locus allele” or similar shall be understood to refer to a variant of the FSL locus which is associated with a male flower phenotype.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome. By modifying the FSL locus polynucleotide sequence in Vitis vinifera and thereby altering FSL polypeptide activity therein, the inventors have found that expression of the FSL locus is essential for male reproductive organ development in flowers of Vitis vinifera. Specifically, the inventors have shown that a knock out of the FSL locus caused the male reproductive organ of flowers in Vitis vinifera to be non-functional, resulting in a phenotypically female flower. This supports the conclusion that expression of the male or hermaphroditic allele of FSL locus is required for normal male reproductive organ development in flowers. In the absence of such expression, or absence of appropriate level of expression, the male reproductive organ will be non-functional or absent, resulting in a phenotypically female flower. As used herein, a “non-functional male reproductive organ” or “non-functional male reproductive part”, or similar, shall be understood to mean a stamen which is incapable of fertilizing a female reproductive organ (i.e., a pistil) of a flower. In some examples, a stamen is non-functional because it contains non-viable pollen i.e., infertile pollen, and/or because it is reflexed and underdeveloped. However, other embodiment in which the stamen is non-functional are contemplated and encompassed herein. A flower which possesses male reproductive parts which are non-functional exhibits “male sterility”.

Based on the finding that the FSL locus and, in particular, expression of the male or hermaphroditic allele of the FSL locus is required for development of functional male reproductive organs in flowers, the present disclosure contemplates the production and use of plants or part thereof having an altered level of FSL polypeptide activity compared to a corresponding wildtype plant or part thereof comprising a wildtype FSL locus or allele thereof. Such altered expression may be used to control flower sex by modifying development of the male reproductive organ or part.

The inventors have characterized the polypeptide sequences encoded by the hermaphroditic, female and male alleles for the FSL locus in Vitis sp., which are set forth in SEQ ID NO: 1-3 respectively. Reference herein to an “FSL polypeptide” is intended to encompass the FSL polypeptide sequences set forth in SEQ ID NO: 1-3, as well as FSL polypeptide sequences having at least 40% identity thereto (e.g., having at least 50%, or at least 60%, or at least 70%, or at least 80%, or at least 90%, or at least 95%, or at least 96%, or at least 97%, or at least 98% or at least 99% identity to the sequence set forth in SEQ ID Nos: 1-3). Also contemplated are orthologues of those sequences which correspond to the particular plant species of interest. In particular examples, activity of an FSL polypeptide encoded by a male or hermaphrodite allele of the FSL locus may be altered, since these alleles are thought to be required for development of functional male reproductive organs.

The term “altered level of FSL polypeptide activity” or similar shall be understood to mean a level of FSL polypeptide activity which is altered (e.g., increased or decreased) relative to the level of activity of FSL polypeptide in a corresponding comparator plant or plant part comprising an FSL locus genotype which confers a male or hermaphrodite flower phenotype. An FSL locus genotype which confers a hermaphrodite flower phenotype may comprise a hermaphrodite allele of the FSL locus e.g., a wildtype hermaphrodite allele of the FSL locus. Likewise, an FSL locus genotype which confers a male flower phenotype may comprise a male allele of the FSL locus e.g., a wildtype male allele of the FSL locus. In accordance with the above example, an “altered level of FSL polypeptide activity” may be a level of FSL polypeptide activity which is altered relative to the activity of an FSL polypeptide encoded by a hermaphrodite or male allele of the FSL locus. In one example, the altered level of FSL polypeptide activity is a decrease in a FSL polypeptide activity relative to the level of activity of FSL polypeptide in a corresponding comparator plant or plant part. In another example, the altered level of FSL polypeptide activity is an absence of FSL polypeptide activity in the corresponding comparator plant or plant part.

Altering the level of FSL polypeptide activity in the plant or plant part may be achieved by modifying a polynucleotide within the FSL locus relative to a corresponding polynucleotide sequence of a wildtype allele of the FSL locus e.g., relative to a corresponding polynucleotide sequence of a wildtype male or hermaphrodite allele of the FSL locus. In one example, a polynucleotide sequence encoding the FSL polypeptide may have an ORF which comprises one or more (e.g., 1, 2, 3, 4, 5, 6, 7, 8, 9, 10 or more) nucleotide additions, deletions or substitutions between positions 153 and 189, such as between positions 155 and 159, relative to the sequence set forth in SEQ ID NO: 6 or 7 (or at one or more corresponding nucleotide positions of the corresponding genomic sequence). For example, the polynucleotide sequence encoding the FSL polypeptide may have an ORF which comprises one or more (e.g., 1, 2, 3, 4, 5, 6, 7, 8, 9, 10 or more) nucleotides deleted between positions 153 and 189 relative to the sequence set forth in SEQ ID NO: 6 or 7 (or at one or more corresponding nucleotide positions of the corresponding genomic sequence). For example, the polynucleotide sequence encoding the FSL polypeptide may have an ORF which comprises one or more T's (e.g., T, TT or TTT) deleted between positions 155 and 159 relative to the sequence set forth in SEQ ID NO: 6 or 7 (or at one or more corresponding nucleotide positions of the corresponding genomic sequence). For example, the polynucleotide sequence encoding the FSL polypeptide may have an ORF which comprises one or more T's (e.g., T, TT or TTT) added between positions 155 and 159 relative to the sequence set forth in SEQ ID NO: 6 or 7 (or at one or more corresponding nucleotide positions of the corresponding genomic sequence). For example, a polynucleotide encoding an FSL polypeptide may be modified such that the open reading frame is interrupted by a stop codon as a result of one or more mutations (e.g., nucleotide substitutions, deletions or additions). In accordance with this example, the modification may result in a FSL polypeptide which is non-functional. In another example, a polynucleotide encoding an FSL polypeptide may be modified so that it is more similar to a female allele of the FSL locus. In this regard, the inventors have determined that a variant of the FSL polypeptide encoded by the female allele of the FSL locus and comprising an amino acid sequence set forth in SEQ ID NO:2, results in a loss of male function i.e., no male reproductive parts develop in a flower from a plant or plant part in which this variant FSL polypeptide is expressed. Relative to FSL polypeptides encoded by the corresponding hermaphrodite and male alleles of the FSL locus (set forth in SEQ ID NOs: 1 and 3 respectively), this FSL polypeptide variant (referred to herein as the female FSL polypeptide) confers a loss of male function. In certain embodiments, FSL polypeptides variants which confer a loss of male function do not comprise a Methionine (M) at a position corresponding to amino acid number 138 of the sequence set forth in SEQ ID NO:2. In other embodiments, FSL polypeptide variants which confer a loss of male function comprise one or more or all of the amino acids at a position corresponding to positions 79, 120, 145, 166, 195, 200, 226, 232 of the sequence set forth in SEQ ID NO:1. Accordingly, altering the level of FSL polypeptide activity in the plant or plant part may be achieved by modifying a polynucleotide encoding the FSL polypeptide to achieve a loss of male function as described herein. Methods of modifying a polynucleotide sequence (e.g., CRISPR, Talon and ZFN) are described in the art and herein.

In another embodiment, altering a level of FSL polypeptide activity in the plant or plant part may be achieved by altering the level of expression (e.g., increasing or decreasing a level of expression) of FSL polypeptide. For example, FSL polypeptide activity may be altered by changes in abundance of a FSL polypeptide expressed in the plant or plant part. For example, the level of expression of FSL polypeptide may be modulated by altering the copy number per cell of the FSL locus or allele thereof encoding the FSL polypeptide. This may be achieved by introducing a synthetic genetic construct comprising the coding sequence and a transcriptional control element that is operably connected thereto and that is functional in the cell. A plurality of transformants may be selected and screened for those with a favourable level of FSL polypeptide activity and/or specificity of expression arising from influences of endogenous sequences in the vicinity of the synthetic construct integration site. A favourable level and pattern of synthetic construct expression is one which results in a substantial modification of FSL phenotype or other phenotype. Alternatively, a population of mutagenized seed or a population of plants from a breeding program may be screened for individual lines with altered FSL polypeptide activity or other phenotype associated with flower sex

In another embodiment, altering a level of FSL polypeptide activity in the plant or plant part may be achieved by modifying the level of a FSL locus transcription product. For example, an RNA interference (RNAi) agent would be used to target a mRNA of the FSL locus, thereby reducing FSL polypeptide activity in the plant or part thereof compared to a corresponding wildtype plant or part thereof.

In another embodiment, altering a level of FSL polypeptide activity in the plant or plant part may be achieved by modifying an interaction of the FSL polypeptide with one or more binding partners thereof e.g., a DNA or protein binding partner involved in a transcription process.

As described herein, altering the activity of an FSL polypeptide may comprise reducing the level of activity. For example, reducing the level of activity of the FSL polypeptide may comprise reducing expression of FSL polypeptide, including the level of expression of functional or biologically active FSL polypeptide. For example, FSL polypeptide activity in the plant or plant part may be reduced by at least 10% relative to a level of FSL polypeptide activity in a corresponding plant or part thereof comprising an FSL locus genotype which confers a male or hermaphrodite flower phenotype. For example, FSL polypeptide activity in the plant or plant part may be reduced by at least 20%, at least 30%, at least 40%, at least 50%, at least 60%, at least 70%, at least 80%, at least 90%, at least 95% relative to a level of FSL polypeptide activity in a corresponding plant or part thereof comprising an FSL locus genotype which confers a male or hermaphrodite flower phenotype. In some examples, altering the activity of FSL polypeptide may comprise completely inhibiting the FSL polypeptide or preventing expression of FSL polypeptide by knocking out the FSL locus or an allele thereof.

The inventors have identified that the FSL locus encodes a “plant AT-rich sequence- and zinc-binding” or “PLATZ” domain. The PLATZ super family of transcription factors have been found to exist only in plants and are likely to be transcription factors. Prior to the present disclosure, PLATZ proteins have not been identified as having an involvement in flower sex determination. In fact, the precise function of PLATZ proteins in plants remains poorly understood. In Vitis vinifera studies, Diaz-Riquelman (2014) found the PLATZ transcription factor family to be upregulated in tendrils which was assumed to related to the cell differentiation taking place during the development of the tendrils. The present inventors have identified a PLATZ domain in the FSL polypeptides of Vitis vinifera at positions 26 to 75 of the sequences set forth in SEQ ID NOs: 1 and 3, and positions 24 to 73 of the sequence set forth in SEQ ID NO: 2. This domain is conserved in each of the female, hermaphroditic and male alleles at the polypeptide level (i.e., 100% identity) Vitus vinifera. The PLATZ domain appears to be essential to FSL polypeptide activity and its role in male reproductive organ development. On this basis, altering the activity of the FSL polypeptide in a plant or plant part to control flower sex may comprise modifying the polynucleotide sequence encoding the PLATZ domain, or post-transcriptional silencing of the FSL mRNA transcript using an RNAi agent targeting a region of the transcript corresponding to the PLATZ domain.

The altered activity of FSL polypeptide in the plant or part thereof e.g., a reduction in FSL polypeptide activity as described herein, may cause a male reproductive part of a flower of the plant to be absent or non-functional. In some examples, the male reproductive part of a flower may be absent due to the altered e.g., reduced, activity of FSL polypeptide. In some examples, the male reproductive part of a flower may be absent due to the altered e.g., reduced, activity of FSL polypeptide resulting from one or mutations in the polynucleotide sequence of the FSL locus or an allele thereof encoding the FSL polypeptide. In other examples, the male reproductive part of a flower may be present but non-functional due to the altered e.g., reduced, activity of FSL polypeptide. A non-functional male reproductive part of a flower may be underdeveloped due to the altered e.g., reduced, activity of FSL polypeptide, causing it to be non-functional. In some examples, a plant or plant part in which the level of FSL polypeptide is altered e.g., reduced, produces flowers which are male sterile.

The inventors have also identified a specific sense mutation SNP in a region of the FSL locus encoding a PLATZ domain which shows 100% concordance between the genotype i.e., male flowers (FSL/fsl or FSL/FSL), female flowers (fsl/fsl) or hermaphroditic flowers (FSL/fsl or FSL/FSL), and flower sex phenotype in Vitis vinifera. As used herein, the SNP may be referred to the “flower sex SNP”. In the female allele of the FSL locus, the flower sex SNP is located at position 621 of the ORF sequence set forth in SEQ ID NO: 5 and comprises an A. In the hermaphrodite allele of the FSL locus, the SNP is located at position 627 of the ORF sequence set forth in SEQ ID NO: 6 and comprises a C. The present inventors contemplate use of this flower sex SNP to determine FSL locus genotype of a plant or plant part and thereby predict its flower sex phenotype, e.g., even before a plant or plant part is sufficiently mature to produce flowers. This SNP may form part of a diagnostic method or test for determining flower sex of a plant or plant part, as described herein. For example, a “female allele of the FSL locus” (or similar term) may have an ORF which comprises a sequence set forth in SEQ ID NO: 5, or a sequence having at least 70% identity thereto (e.g., having at least 75%, or at least 80%, or at least 85%, or at least 90%, or at least 95%, or at least 96%, or at least 97%, or at least 98% or at least 99% identity to the sequence set forth in SEQ ID NO: 5), provided that the nucleotide position corresponding to position 621 of the ORF sequence set forth in SEQ ID NO: 5 is an A. For example, a “hermaphrodite allele of the FSL locus” or a “male allele of the FSL locus” (or similar terms) may have an ORF which comprises a sequence set forth in SEQ ID NO: 6, or a sequence having at least 70% identity thereto (e.g., having at least 75%, or at least 80%, or at least 85%, or at least 90%, or at least 95%, or at least 96%, or at least 97%, or at least 98% or at least 99% identity to the sequence set forth in SEQ ID NO: 5), provided that the nucleotide position corresponding to position 627 of the ORF sequence set forth in SEQ ID NO: 6 is a C. By determining the genotype of a plant or plant part at the flower sex SNP within the FSL locus (using standard molecular techniques), flower sex may be predicted or determined.

As stated herein, the terms “FSL locus”, “FSL locus alleles” and “FSL polypeptides” are intended to encompass orthologous FSL locus sequences, orthologous FSL allelic sequences (including orthologues of the male, female and hermaphrodite FSL allele sequences) and orthologous FSL polypeptide sequences to those exemplified for Vitis sp. The orthologues will preferably correspond to the particular plant species being produced. “Orthologous” genes, loci, alleles or polypeptides are homologues that have diverged after a speciation event. Although sequence variations may arise between orthologous genes, loci, alleles or polypeptides once two species have diverged, orthologues may maintain the same or substantially the same function to that of the ancestral gene, loci, allele or polypeptide from which they have evolved.

Thus, orthologous FSL locus sequences, including male, female and hermaphrodite alleles thereof, will be understood to include FSL locus sequences derived from plant species other than Vitis vinifera which have common ancestry to the sequences set forth in SEQ ID NOs: 4-7 and which perform the same or similar function in the respective plant species. Likewise, orthologous FSL polypeptides will be understood to include FSL polypeptide sequences derived from plant species other than Vitis vinifera which have common ancestry to the sequences set forth in SEQ ID NOs: 1-3 and which perform the same or similar function in the respective plant species.

The term “wildtype” is generally understood to mean a typical or common form of a gene, loci, allele, polypeptide or phenotype that occurs in an organism (or within a given population) in nature. Unless specifically stated otherwise, the term “wildtype” shall be understood to have its regular meaning. However, in the context of the FSL locus, the term “wildtype” is used herein to delineate between naturally-occurring or unmodified forms of FSL locus alleles and modified or altered counterparts of the disclosure. In this regard, the inventors have shown that sex-specific alleles of the FSL locus exist, i.e., a male-specific FSL locus allele, a female-specific FSL locus allele, and a hermaphrodite-specific FSL locus allele. In order to delineate between naturally-occurring or unmodified forms of the respective sex-specific FSL locus alleles and modified or altered counterparts of the disclosure, the term “wildtype” has also been used to denote the respective naturally-occurring or unmodified allelic forms. Accordingly, as used herein, the term “wildtype male FSL locus allele”, “wildtype male allele of the FSL locus” or similar shall be understood to refer to the naturally-occurring or unmodified male allele of the FSL locus. Similarly, the term “wildtype female FSL locus allele”, “wildtype female allele of the FSL locus” or similar as used herein shall be understood to refer to the naturally-occurring or unmodified female allele of the FSL locus. Similarly, the term “wildtype hermaphrodite FSL locus allele”, “wildtype hermaphrodite allele of the FSL locus” or similar as used herein shall be understood to refer to the naturally-occurring or unmodified hermaphrodite allele of the FSL locus. In accordance with an example in which the plant species is Vitis vinifera, the wildtype alleles for female, hermaphrodite and male may have ORFs which comprise the sequences set forth in SEQ ID NOs: 5-7, respectively. However, it will be understood that sequences of female, hermaphrodite and male alleles of the FSL locus may vary within a particular species (e.g., variation between different populations), as well as between species (e.g., orthologues). Accordingly, it will be appreciated that reference to wildtype in the context of female, hermaphrodite and male alleles of the FSL locus may also encompass ORF sequences which are at least 65%, at least 70%, at least 75%, at least 80%, at least 85%, at least 90%, at least 95%, at least 96%, at least 97%, at least 98%, or at least 99% identical to the sequences set forth in SEQ ID NOs: 5-7, respectively.

The inventors have also found that the sex-specific alleles of the FSL locus encode FSL polypeptides with varying sequences. Accordingly, reference herein to the term “wildtype” in the context of FSL polypeptides refers to the naturally-occurring or unmodified FSL polypeptide variant encoded by the wildtype hermaphrodite, female, and male alleles of the FSL locus, respectively, as described herein. In accordance with an example in which the plant species is Vitis vinifera, the FSL polypeptides encoded by the wildtype hermaphrodite, female, and male alleles of the FSL locus may comprise the sequences set forth in SEQ ID NOs: 1-3, respectively. However, as with the FSL locus and sex-specific alleles thereof, it will be understood that the FSL polypeptide sequences encoded by the wildtype hermaphrodite, female, and male alleles of the FSL locus may vary within a particular species (e.g., variation between different populations), as well as between different species (e.g., FSL polypeptide orthologues). Accordingly, it will be appreciated that reference to FSL polypeptide sequences encoded by the wildtype hermaphrodite, female, and male alleles of the FSL locus (collectively “wildtype FSL polypeptides”) may also encompass sequences which are at least 50%, at least 60%, at least 70%, at least 80%, at least 85%, at least 90%, at least 95%, at least 96%, at least 97, at least 98%, at least 99% identical to the amino acid sequences set forth in SEQ ID NOs:1-3, respectively.

The term “wildtype” as used in the context of a plant or part thereof of the disclosure, will be understood to mean a plant or plant part in which the FSL locus or the FSL polypeptide has not been modified i.e., a plant or plant part comprising the FSL locus or an allele thereof as it occurs in nature in that plant species.

Terms such as “modifying”, “modify”, “modifies” or similar, as used herein in the context of modifying the FSL locus or an allele thereof, shall be understood to mean introducing one or more physical changes to the FSL locus sequence or an allele thereof, including nucleotide substitutions, additions and/or deletions, relative to a reference FSL locus sequence e.g., the sequence of a wildtype male or hermaphrodite allele of the FSL locus. Exemplary modifications are described herein. Modification to the FSL locus sequence can be achieved using any means known in the art for modifying nucleic acids including, for example, random and site-directed mutagenesis, transgenic expression, CRISPR, TALON and/or ZFN technologies as described in the art or herein. The one or more changes to the FSL locus sequence or an allele thereof preferably results in one or more changes to the amino acid sequence of the FSL polypeptide encoded thereby e.g., one or more amino acid additions, deletions or substitutions relative to the FSL polypeptide sequence encoded by the corresponding unmodified FSL locus sequence or an allele thereof. Accordingly, the altered level of activity of the FSL polypeptide may be achieved by introducing one or more changes to the sequence of the FSL locus or an allele thereof. Preferably FSL polypeptide activity is reduced or abrogated by modifying the sequence of the FSL locus or an allele thereof and the corresponding FSL polypeptide encoded thereby. However, in some alternative examples, FSL polypeptide activity may be increased by modifying the sequence of the FSL locus or an allele thereof e.g., by introducing one or more copies of a male allele or a hermaphrodite allele of the FSL locus to a plant or plant part using recombinant methods.

As used herein, the term “heterozygous” refers to the presence of different alleles (forms of a given gene) at a particular gene locus. Thus, reference to a “heterozygote” refers to a diploid or polyploid individual plant cell or plant having different alleles (forms of a given gene) present at least at one locus.

As used herein, the term “homozygous” refers to the presence of identical alleles at one or more loci in homologous chromosomal segments. Thus, reference herein to a “homozygote” refers to an individual plant cell or plant having the same alleles at one or more loci.

As used herein, the term “a progeny plant capable of exhibiting a desired flower sex phenotype on the basis of the FSL locus genotype” shall be understood to mean a progeny that has an FSL locus genotype which confers a flower sex phenotype of interest. In some examples, a “progeny plant which capable of exhibiting a desired flower sex phenotype on the basis of the FSL locus genotype” is actually exhibiting the flower sex phenotype of interest i.e., the progeny plant is either in flower or a flower is in a stage of development. In other examples, a “progeny plant which capable of exhibiting a desired flower sex phenotype on the basis of the FSL locus genotype” is not in flower e.g., at the time of a section step. This may be because the progeny plant is immature and not yet capable of producing flowers, or because environmental conditions are not conducive to flowering.

### Seedless-Ness

As used herein, the term “fruit” shall be understood to mean a seed bearing structure developed from the ovary of angiosperm flowers, typically following fertilisation with viable pollen.

As used herein, the term “seed” is intended to encompass “mature seed” as well as “developing seed” which occurs after fertilisation and prior to seed dormancy being established and before harvest.

The term “seedless”, as used herein in the context of fruit, may refer to the complete absence of hard seeds in the (mature) fruit (i.e. “no seeds set” as a result of parthenocarpy) and/or a significant reduction in total seed number (i.e. “reduced seed set”) and/or an arrest of seed development in the early stages (e.g., as a result of stenospermocarpy), so that there is a significant reduction in the eventual number of fully developed seeds, whereby a significant reduction refers to a reduction to at least 40% of the wild type, preferably a reduction to at least 50%, 60%, 70%, 80%, 90%, 95% or 98%, most preferably a reduction to 100% of the wild type (i.e. completely seedless). Stenospermocarpic seedless-ness occurs through a biological process whereby a flower is fertilised and the seed starts to develop, but development of the seed is aborted at an early stage leaving a ‘seed trace’ in the fruit. Accordingly, the term “seedless” as used herein encompasses a phenotype in which fruit contains seed trace or one or more soft seeds which are remnants of the aborted undeveloped seed.

### Dwarf Stature

The present disclosure provides novel plants or plant parts which produce seedless fruit, wherein the plants or plant parts have altered e.g., reduced, FSL polypeptide activity as described herein, and a polynucleotide which confers dwarf stature.

The present disclosure provides novel plants or plant parts which produce seedless fruit, wherein the plants or plant parts comprise an FSL locus which is homozygous for the FSL locus female allele (f/f) as described herein, and a polynucleotide which confers dwarf stature. Plants or plant parts which are homozygous for the FSL locus female allele (f/f) may be identified using the flower sex SNP as described herein.

As referred to herein, a “dwarf” plant will be understood to mean an individual plant or plant variety of a particular species which is shorter in height relative to the average (normal) height for the particular species. Thus, “dwarf stature” is short stature.

The literature is replete with the development of dwarf plants including genes and means for achieving a dwarf stature. Any polynucleotide known in the art for conferring dwarf stature to a plant is contemplated herein.

In one example, the polynucleotide that confers dwarf stature to the plant is altered relative to the corresponding wildtype or naturally-occurring polynucleotide sequence.

The development of a dwarf grapevine with a rapid flowering phenotype, referred to as “microvines”, has been described previously by the inventors (Boss and Thomas, (2002) Nature, 416(6883):847-850). The previously reported “microvine” phenotype is based on a variant of the Gibberellic Acid Insensitive (VvGAI1) gene comprising a SNP (T to A mutation) in the translated region at position 231 of the normal VvGAI1 gene. The point mutation present in the variant VvGAI1 gene converts a leucine residue of the conserved DELLA domain into histidine, thereby altering the gibberellic acid (GA) response properties of the plant. The variant GAI1 gene causes a dwarf stature and rapid flowering phenotype when present in either heterozygous (GAI1/gai1) or homozygous (gai1/gai1) state. Accordingly, in some examples, reference herein to a “mutated gibberellic acid insensitive (GAI1) gene” or similar in the context of a plant, or plant progeny, propagative material or fruit thereof, of the disclosure shall be understood to mean a mutated GAI1 gene variant which confers dwarf stature and a rapid flowering phenotype as previously described in Boss and Thomas (2002), the full content of which is incorporated herein by reference, or other mutated GAI1 gene variant which similarly prevents the GAI1 protein from responding to GA signaling.

In one example, the polynucleotide that confers dwarf stature is a variant of the GAI1 gene or a fragment thereof. The variant of the GAI1 gene may encode a “variant GAI1 protein”. In one example, the variant of the GAI1 gene or fragment thereof comprises one or more mutations in a region encoding the DELLA domain. For example, the one or more mutations in the region encoding the DELLA domain of the GAI1 protein may alter GA response properties of the plant or plant part e.g., as in the microvine. For example, the one or more mutations in the DELLA domain may prevent the plant or plant part from responding to GA signalling. Accordingly, in some examples, the plant or plant part comprising a variant of the GAI1 gene or a fragment thereof does not respond, or responds poorly, to GA signalling. The one or more mutations may be selected from amino acid substitutions, deletions or additions. In one example, the variant GAI1 protein may comprise a sequence set forth in SEQ ID NO: 8 with a Leu to His substitution at position 38 thereof, or a sequence having at least 85% identity, or at least 90% identity, or at least 95% identity, or at least 96% identity, or at least 97% identity, or at least 98% identity, or at least 99% identity to the sequence set forth in SEQ ID NO: 8 provided that the Leu of the DELLA domain corresponding to position 38 of SEQ ID NO: 8 is substituted with a larger basic residue e.g., His. In one example, the variant GAI1 protein comprises a sequence which is at least 99.1%, at least 99.2%, at least 99.3%, at least 99.4%, at least 99.5%, at least 99.6%, at least 99.7%, or at least 99.8% identical to the sequence set forth in SEQ ID NO: 8, provided that the Leu of the DELLA domain corresponding to position 38 of SEQ ID NO: 8 is substituted with a larger basic residue e.g., His.

The variant GAI1 protein may comprise the sequence set forth in SEQ ID NO: 9. In accordance with this example, the plant or plant part may be a “microvine” as described in Boss and Thomas, (2002) Nature, 416(6883):847-850 which has an altered level of FSL polypeptide activity as described herein. In other examples, the DELLA domain may be, altered truncated or completely deleted from the GAI1 gene or fragment thereof e.g., as a result of the one or more mutations. The one or more mutations preferably result in a non-functional GAI1 gene.

The polynucleotide which confers dwarf status e.g., the variant of the GAI1 gene or fragment thereof, may be present in a homozygous (GAI1/GAI1) state or a heterozygous (GAI1/Gai1) state.

Also contemplated is an plant or plant part in which the GAI1 protein or the DELLA domain thereof is silenced e.g., post-transcriptionally silenced using an RNAi agent. In accordance with this example, the polynucleotide which confers a dwarf stature to the plant may be an RNAi agents targeting a mRNA transcript of the GAI1 protein e.g., such as corresponding to the DELLA domain. RNAi agents are described herein.

In each of the foregoing examples describing a plant or plant part having an altered level of FSL polypeptide activity and a polynucleotide which confers dwarf stature, the plant or part thereof may produce parthenocarpic seedless fruit when flowers are unpollinated and fruit containing seeds when flowers are pollinated with viable pollen.

### Stenospermocarpy

The present disclosure also contemplates novel plants and plant parts that produce seedless fruit, wherein said plant or plant parts comprise: an altered e.g., reduced, level of FSL polypeptide activity as described herein; a polynucleotide that confers dwarf stature as described herein; and a polynucleotide that confers stenospermocarpy.

The present disclosure also contemplates novel plants and plant parts that produce seedless fruit, wherein said plant or plant parts comprise: an FSL locus which is homozygous for the FSL locus female allele (f/f) as described herein; a polynucleotide that confers dwarf stature as described herein; and a polynucleotide that confers stenospermocarpy.

“Stenospermocarpy” is the biological mechanism that produces seedlessness in some fruits, notably many table grapes. In “stenospermocarpic” seedless fruits, normal pollination and fertilization are still required to ensure that the fruit ‘sets’, i.e. continues to develop on the plant; however subsequent abortion of the embryo that began growing following fertilization leads to a near seedless condition. The remains of the undeveloped seed are visible in the fruit. Table grape varieties that are “seedless” produce “seedless” fruit due to stenospermocarpy where the flower is fertilised and the seed starts to develop but stops development at an early stage leaving a seed trace in the fruit. In some examples, fruit produced from plants or plant parts of the present disclosure is “seedless” having a seedless-ness phenotype consistent with that exhibited by fruit produced from fertilized female ovules of stenospermocarpic plants. Frequently, stenospermocarpic fruit may contain one or more ‘soft seeds’ which are the remnants of the arrested fertilised seed.

To be differentiated from stenospermocarpy is parthenocarpy. “Parthenocarpy” is generally understood in the art, and also to be understood in connection with the present disclosure, to describe the development of fruits without fertilization of the female ovule. “Parthenocarpy” literally means “virgin fruit”. As the pollination process is not required for producing fruits, no seed ever develops. In this sense, “parthenocarpic” fruit exhibit true seedless-ness.

Any polynucleotide known in the art for conferring stenospermocarpy to plants is contemplated herein. In some example, the polynucleotide that confers stenospermocarpy to the plant or part thereof may be altered relative to the corresponding wildtype or naturally-occurring gene. In one particular example, the polynucleotide that confers stenospermocarpy to the plant or plat part is a variant of the Vitis vinifera MADS-box protein 5 (VvMADS5) locus. The variant or mutated VvMADS5 locus (also known as VviAGL11) is known to be associated with stenospermocarpy (SDL1) seedless-ness phenotype in Vitus sp. when present in either the heterozygous or homozygous state. The mutation in this variant of VvMADS5 results in a G to T substitution at 590 bp of the coding sequence resulting in an Arg197Leu substitution (Royo et al., 2018). Accordingly, reference herein to “a mutated VvMADS5 gene associated with stenospermocarpy”, “variant VvMADS5 locus” or similar shall be understood to encompass the mutant VvMADS5 gene described in Royo et al., (2018), the full content of which is incorporated herein by reference.

In one example, the polynucleotide that confers stenospermocarpy is a variant of the VvMADS5 locus. The VvMADS5 locus encoding the VvMADS5 protein (i.e., endogenous or non-variant protein) may comprise the amino acid sequence set forth in SEQ ID NO: 10, and the variant VvMADS5 protein may comprise a substitution of the Arg at position 197 of the sequence set forth in SEQ ID NO: 10 with a hydrophobic amino acid e.g., Leu (R197L). In one example, the variant VvMADS5 locus encodes a variant VvMADS5 protein comprising an amino acid sequence set forth in SEQ ID NO: 11, or a sequence having at least 80% identity thereto (e.g., having at least 85% identity, or at least 90% identity, or at least 95% identity, or at least 96% identity, or at least 97% identity, or at least 98% identity, or at least 99% identity to the sequence set forth in SEQ ID NO: 11) provided that the amino acid at position 197 relative to SEQ ID NO: 11 is a hydrophobic amino acid e.g., Leu. For example, the variant VvMADS5 locus may encode a variant VvMADS5 protein comprising an amino acid sequence which is at least 99.1%, at least 99.2%, at least 99.3%, at least 99.4%, or at least 99.5% identical to the sequence set forth in SEQ ID NO: 11 provided that the amino acid at position 197 relative to SEQ ID NO: 11 is a hydrophobic amino acid e.g., Leu. In one particular example, the variant VvMADS5 locus encodes a variant VvMADS5 protein comprising the amino acid sequence set forth in SEQ ID NO: 11 e.g., as described in Royo et al., 2018. In other examples, the VvMADS5 protein be truncated or completely deleted from the plant or plant part e.g., as a result of the one or more mutations to the VvMADS5 locus. The one or more mutations preferably result in a non-functional VvMADS5 protein.

The polynucleotide which confers stenospermocarpy e.g., the variant VvMADS5 locus encoding the variant VvMADS5 protein as described herein, may be present in a homozygous or a heterozygous state.

In another example, the VvMADS5 protein is silenced e.g., post-transcriptionally silenced. In accordance with this example, the polynucleotide which confers stenospermocarpy to the plant may be an RNAi agent targeting a mRNA transcript encoded by the VvMADS5 locus.

In each of the foregoing examples describing a plant or plant part which further comprises a polynucleotide that confer stenospermocarpy, the plant produces parthenocarpic seedless fruit when flowers are unpollinated and stenospermocarpic fruit when flowers are pollinated with viable pollen.

### Polypeptides

As used herein the term “FSL polypeptide” shall be understood to mean a polypeptide encoded by the FSL locus or an allele thereof as described herein, the activity of which has been shown by the inventors to be responsible for flower sex. Specifically, the inventors have shown that the FSL polypeptide encoded by male and hermaphrodite alleles of the FSL locus is responsible for the development of the male reproductive organ of flowers. As used herein, the term “FSL polypeptide” generally relates to a protein family which shares a high level of primary sequence identity to the polypeptide sequences set forth in SEQ ID NO: 1-3, for example FSL polypeptide sequences having at least 40% identity sequences set forth in SEQ ID NO: 1-3 (e.g., having at least 50%, or at least 60%, and preferably at least 70%, or at least 80%, or at least 90%, or at least 95%, or at least 96%, or at least 97%, or at least 98% or at least 99% identity to the sequence set forth in SEQ ID NOs: 1-3). Also contemplated are orthologues of those sequences which correspond to the particular plant species of interest. The present inventors have determined that the altered level of activity of certain variants of the FSL polypeptide family, when expressed in a plant, cause altered flower sex phenotype. An example of a variant comprises an amino acid sequence provided as SEQ ID NO:2, encoded by the female allele of the FSL locus. Relative to FSL polypeptides encoded by the corresponding hermaphrodite and male alleles of the FSL locus (set forth in SEQ ID NOs: 1 and 3 respectively), this FSL polypeptide variant (referred to herein as the female FSL polypeptide) confer a loss of male function i.e., a functional male reproductive part does not develop in the flower. In certain embodiments, FSL polypeptide variants which confer a loss of male function do not comprise a Methionine (M) at a position corresponding to amino acid number 138 of the sequence set forth in SEQ ID NO:2. In other embodiments, FSL polypeptide variants which confer a loss of male function comprise one or more or all of the amino acids at a position corresponding to positions 79, 120, 145, 166, 195, 200, 226, 232 of the sequence set forth in SEQ ID NO:1. In particular examples, activity of an FSL polypeptide encoded by a male or hermaphrodite allele of the FSL locus may be altered to confer the loss of male function by modifying one or more of the amino acids as described herein, since these alleles are thought to be required for development of functional male reproductive organs.

The inventors have identified that the FSL polypeptide includes a “plant AT-rich sequence- and zinc-binding” or “PLATZ” domain. The PLATZ super family of transcription factors have been found to exist only in plants and are likely to be transcription factors. Prior to the present disclosure, PLATZ proteins have not been identified as having an involvement in flower sex determination. The present inventors have identified a PLATZ domain in the FSL polypeptides of Vitis vinifera at positions 26 to 75 of the sequences set forth in SEQ ID NOs: 1 and 3, and positions 24 to 73 of the sequence set forth in SEQ ID NO: 2. As such, reference herein to a “PLATZ domain” is intended to encompass the amino acid sequences set forth from position 26 to 75 of the sequences set forth in SEQ ID NOs: 1 and 3, and position 24 to 73 of the sequence set forth in SEQ ID NO: 2, as well PLATZ domains of FSL polypeptides having at least 40% identity to those sequences (e.g., having at least 80%, or at least 90%, or at least 95%, or at least 96%, or at least 97%, or at least 98% or at least 99% identity to the PLATZ domains within the sequence set forth in SEQ ID NOs: 1-3). Also contemplated are orthologues of those sequences which correspond to the particular plant species of interest. In some examples, one or more mutations may be introduced to the PLATZ domain of the FSL polypeptide to alter FSL polypeptide activity e.g., to confer a loss of male function.

Reference herein to a “variant GAI1 protein” shall be understood to mean a protein or polypeptide encoded by a variant of the GAI1 gene or fragment thereof comprising one or more mutations, such as in a region encoding the DELLA domain, as described herein. The one or more mutations in the region encoding the DELLA domain of the GAI1 protein preferably alter GA response properties of a plant or plant part which expresses the variant GAI1 protein. The one or more mutations may be selected from amino acid substitutions, deletions or additions. Exemplary “variant GAI1 proteins” include, but are not limited to, those polypeptides that comprise a sequence set forth in SEQ ID NO: 8 with a Leu to His substitution at position 38 thereof, or a sequence having at least 80% identity thereto (e.g., having at least 85% identity, or at least 90% identity, or at least 95% identity, or at least 96% identity, or at least 97% identity, or at least 98% identity, or at least 99% identity to the sequence set forth in SEQ ID NO: 8) provided that the Leu of the DELLA domain corresponding to position 38 of SEQ ID NO: 8 is substituted with a larger basic residue e.g., His. In preferred examples, the variant GAI1 protein comprises a sequence which is at least 99.1%, at least 99.2%, at least 99.3%, at least 99.4%, at least 99.5%, at least 99.6%, at least 99.7%, or at least 99.8% identical to the sequence set forth in SEQ ID NO: 8, provided that the Leu of the DELLA domain corresponding to position 38 of SEQ ID NO: 8 is substituted with a larger basic residue e.g., His. One preferred variant GAI1 protein may comprise the sequence set forth in SEQ ID NO: 9.

Reference herein to a “variant VvMADS5 protein” shall be understood to mean any protein or polypeptide encoded by a variant VvMADS5 locus or fragment thereof provided that the polypeptide differs in sequence to the wildtype or naturally-occurring VvMADS5 protein. Exemplary “variant VvMADS5 proteins” include, but are not limited to, those polypeptides comprising an amino acid sequence set forth in SEQ ID NO: 11, or a sequence having at least 80% identity thereto (e.g., having at least 85% identity, or at least 90% identity, or at least 95% identity, or at least 96% identity, or at least 97% identity, or at least 98% identity, or at least 99% identity to the sequence set forth in SEQ ID NO: 11) provided that the amino acid at position 197 relative to SEQ ID NO: 11 is a hydrophobic amino acid e.g., Leu. For example, the variant VvMADS5 locus may encode a variant VvMADS5 protein comprising an amino acid sequence which is at least 99.1%, at least 99.2%, at least 99.3%, at least 99.4%, or at least 99.5% identical to the sequence set forth in SEQ ID NO: 11 provided that the amino acid at position 197 relative to SEQ ID NO: 11 is a hydrophobic amino acid e.g., Leu. One preferred variant VvMADS5 protein comprises the amino acid sequence set forth in SEQ ID NO: 11 e.g., as described in Royo et al., 2018.

As used herein a “biologically active fragment” of a FSL polypeptide is a portion of a FSL polypeptide of the disclosure which maintains the activity of a full-length FSL polypeptide. Biologically active fragments as used herein exclude the full-length polypeptide. Biologically active fragments can be any size portion as long as they maintain the defined activity. In one example, a biologically active fragment of the FSL polypeptide is the PLATZ domain. Preferably, the biologically active fragment maintains at least 10% of the activity of the full length polypeptide.

The terms “polypeptide” and “protein” are generally used interchangeably herein.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over their entire length, and the extent of identity is determined over the full length of the reference sequence. The polypeptide or class of polypeptides may have the same enzymatic activity as, or a different activity than, or lack the activity of, the reference polypeptide. Preferably, the FSL polypeptide, the activity of which is altered in accordance with the present disclosure, has an activity which is at least 10% less (e.g., at least 20% less, or at least 30% less, or at least 40% less, or at least 50% less, or at least 60% less, or at least 70% less, or at least 80% less, or at least 90% less) than the activity of the reference FSL polypeptide (e.g., a FSL polypeptide encoded by a wildtype allele of the FSL locus as described herein). In some examples, the altered level of FSL polypeptide activity mean an absence of FSL activity.

As used herein a “biologically active fragment” is a portion of a polypeptide of the disclosure which maintains a defined activity of a full-length reference polypeptide. Biologically active fragments as used herein exclude the full-length polypeptide. Biologically active fragments can be any size portion as long as they maintain the defined activity.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides defined herein can be prepared by introducing appropriate nucleotide changes into a nucleic acid defined herein, or by in vitro synthesis of the desired polypeptide. Such mutants include for example, deletions, insertions, or substitutions of residues within the amino acid sequence. A combination of deletions, insertions and substitutions can be made to arrive at the final construct, provided that the final polypeptide product possesses the desired characteristics.

Mutant (altered or variant) polypeptides can be prepared using any technique known in the art, for example, using directed evolution or rational design strategies (see below). Products derived from mutated/altered DNA can readily be screened using techniques described in the art and herein to determine if they possess FSL polypeptide activity and influence development of male reproductive parts flowers.

In designing amino acid sequence mutants, the location of the mutation site and the nature of the mutation will depend on characteristic(s) to be modified. The sites for mutation can be modified individually or in series for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide removed and a different residue inserted in its place. The sites of greatest interest for substitutional mutagenesis to inactivate enzymes include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In a preferred embodiment a mutant/variant polypeptide has only, or not more than, one or two or three or four amino acid changes when compared to a naturally occurring polypeptide. Mutants with desired activity may be engineered using standard procedures in the art such as by performing random mutagenesis, targeted mutagenesis, or saturation mutagenesis on known genes of interest, or by subjecting different genes to DNA shuffling.

Also contemplated are FSL polypeptides of the disclosure e.g., having altered FSL activity, which have been differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. Such polypeptides may be post-translationally modified in a cell, for example by phosphorylation, which may modulate their activity. These modifications may serve to increase the stability and/or bioactivity of the FSL polypeptides of the disclosure.

### Polynucleotides

The terms “polynucleotide”, and “nucleic acid” are used interchangeably. They refer to a polymeric form of nucleotides of any length, either deoxyribonucleotides or ribonucleotides, or analogs thereof. A polynucleotide of the disclosure may be of genomic, cDNA, semisynthetic, or synthetic origin, double-stranded or single-stranded and by virtue of its origin or manipulation: (1) is not associated with all or a portion of a polynucleotide with which it is associated in nature, (2) is linked to a polynucleotide other than that to which it is linked in nature, or (3) does not occur in nature. The following are non-limiting examples of polynucleotides: coding or non-coding regions of a gene or gene fragment, exons, introns, messenger RNA (mRNA), transfer RNA (tRNA), ribosomal RNA (rRNA), ribozymes, cDNA, recombinant polynucleotides, plasmids, vectors, isolated DNA of any sequence, isolated RNA of any sequence, chimeric DNA of any sequence, nucleic acid probes, and primers. For in vitro use, a polynucleotide may comprise modified nucleotides such as by conjugation with a labeling component.

As used herein, an “isolated polynucleotide” refers to a polynucleotide which has been separated from the polynucleotide sequences with which it is associated or linked in its native state, or a non-naturally occurring polynucleotide.

As used herein, the term “gene” is to be taken in its broadest context and includes the deoxyribonucleotide sequences comprising the transcribed region and, if translated, the protein coding region, of a structural gene and including sequences located adjacent to the coding region on both the 5′ and 3′ ends for a distance of at least about 2 kb on either end and which are involved in expression of the gene. In this regard, the gene includes control signals such as promoters, enhancers, termination and/or polyadenylation signals that are naturally associated with a given gene, or heterologous control signals, in which case, the gene is referred to as a “chimeric gene”. The sequences which are located 5′ of the protein coding region and which are present on the mRNA are referred to as 5′ non-translated sequences. The sequences which are located 3′ or downstream of the protein coding region and which are present on the mRNA are referred to as 3′ non-translated sequences. The term “gene” encompasses both cDNA and genomic forms of a gene. A genomic form or clone of a gene contains the coding region which may be interrupted with non-coding sequences termed “introns”, “intervening regions”, or “intervening sequences.” Introns are segments of a gene which are transcribed into nuclear RNA (nRNA). Introns may contain regulatory elements such as enhancers. Introns are removed or “spliced out” from the nuclear or primary transcript; introns are therefore absent in the mRNA transcript. A gene which contains at least one intron may be subject to variable splicing, resulting in alternative mRNAs from a single transcribed gene and therefore polypeptide variants. A gene in its native state, or a chimeric gene may lack introns. The mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, “chimeric DNA” refers to any DNA molecule that is not naturally found in nature; also referred to herein as a “DNA construct” or “genetic construct”. Typically, a chimeric DNA comprises regulatory and transcribed or protein coding sequences that are not naturally found together in nature. Accordingly, chimeric DNA may comprise regulatory sequences and coding sequences that are derived from different sources, or regulatory sequences and coding sequences derived from the same source, but arranged in a manner different than that found in nature. The open reading frame may or may not be linked to its natural upstream and downstream regulatory elements. The open reading frame may be incorporated into, for example, the plant genome, in a non-natural location, or in a replicon or vector where it is not naturally found such as a bacterial plasmid or a viral vector. The term “chimeric DNA” is not limited to DNA molecules which are replicable in a host, but includes DNA capable of being ligated into a replicon by, for example, specific adaptor sequences.

The term “genetically modified”, “genetic modification”, “modified” (in the context of a nucleic acid sequence) and variations thereof, is a broader term that includes introducing a gene into a cell by transformation or transduction, mutating a gene in a cell and genetically altering or modulating the regulation of a gene in a cell, or the progeny of any cell modified as described above.

A “recombinant polynucleotide” of the disclosure refers to a nucleic acid molecule which has been constructed or modified by artificial recombinant methods. The recombinant polynucleotide may be present in a cell of a plant or part thereof in an altered amount or expressed at an altered rate (e.g., in the case of mRNA) compared to its native state. In one embodiment, the polynucleotide is endogenous to the plant or part thereof and its expression is altered by recombinant means, for example, an exogenous control sequence is introduced upstream of an endogenous gene of interest to enable the transformed plant or part thereof to express the polypeptide encoded by the gene, or a deletion is created in a gene of interest by ZFN, Talen or CRISPR methods.

A “recombinant polynucleotide” of the disclosure includes polynucleotides which have not been separated from other components of the cell-based or cell-free expression system, in which it is present, and polynucleotides produced in said cell-based or cell-free systems which are subsequently purified away from at least some other components. The polynucleotide can be a contiguous stretch of nucleotides or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

A polynucleotide of, or useful for, the present disclosure may selectively hybridise, under stringent conditions, to a polynucleotide defined herein. As used herein, stringent conditions are those that: (1) employ during hybridisation a denaturing agent such as formamide, for example, 50% (v/v) formamide with 0.1% (w/v) bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (2) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS, and/or (3) employ low ionic strength and high temperature for washing, for example, 0.015 M NaCl/0.0015 M sodium citrate/0.1% SDS at 50° C.

Polynucleotides of the disclosure may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described herein).

### Nucleic Acid Constructs

The present disclosure includes nucleic acid constructs comprising the polynucleotides useful for preparing plants and plant parts of the disclosure, and vectors and host cells containing these, methods of their production and use, and uses thereof. The present disclosure refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some Pol III promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable.

In a preferred embodiment, if a constitutive promoter is used it results in high levels of mRNA transcribed from the exogenous polynucleotide such that the level of a specific NAC transcription factor that is produced in at least a part of the plant is at least about 5 fold or 10 fold or 15 fold or 20 fold higher when compared to an isogenic wheat plant lacking the exogenous polynucleotide. Non-limiting methods for assessing promoter activity are disclosed by Medberry et al. (1992, 1993), Sambrook et al. (1989, supra) and U.S. Pat. No. 5,164,316. Examples of constitutive promoters which may result in these levels of mRNA production include, but are not limited to, the core promoter of the Rsyn7 promoter and other constitutive promoters disclosed in WO99/43838 and U.S. Pat. No. 6,072,050; the core CaMV 35S promoter (Odell et al., 1985) or its enhanced versions; rice actin (McElroy et al., 1990); ubiquitin (Christensen et al., 1989 and 1992); pEMU (Last et al., 1991); MAS (Velten et al., 1984); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

“Selective expression” as used herein refers to expression almost exclusively in specific organs of, for example, the plant, such as, for example, egg-cell, sperm cell, ovule, pollen, stamen, anthers, endosperm, embryo, leaves, or root. In a preferred embodiment, a promoter is expressed selectively or preferentially in flowers or flower parts of a grapevine plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

“Inducible promoters” selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, infection or drought, phytohormones, wounding, or chemicals such as ethanol, jasmonate, salicylic acid, or safeners. As used herein, a “plant stress inducible promoter” is any inducible promoter that is functional in a wheat plant, and hence this term is not limited to promoters derived from a plant.

Suitable inducible promoters for use in expressing the above-described nucleic acids in a plant include promoters that are induced by physiological or environment conditions which trigger or are associated with flowering. Suitable inducible promoters are known in the art and contemplated herein.

Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present disclosure may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for PolI or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred sequence which can increase or maintain mRNA stability and prevent inappropriate initiation of translation as for example described by Joshi (1987).

### Polynucleotides for Reducing Expression of Genes

An altered level of FSL polypeptide activity in accordance with the present disclosure may also be achieved through post-transcriptional silencing of the messenger RNA (mRNA) transcribed from the FSL locus using RNA interference (RNAi). The term “RNA interference” or “RNAi” refers generally to RNA-dependent silencing of gene expression initiated by double stranded RNA (dsRNA) molecules in a cell's cytoplasm. The dsRNA molecule reduces or inhibits transcription products of a target nucleic acid sequence, thereby silencing the gene or reducing expression of that gene. A “double stranded RNA” or “dsRNA” refers to a RNA molecule having a duplex structure and comprising an “antisense sequence” or “guide strand” and a “sense sequence” or “passenger strand” which are of similar length to one another. The cognate antisense and sense sequences can be in a single RNA strand or in separate RNA strands. The antisense sequence will be substantially complementary to a target sequence, which in the present case, is a region of the FSL polypeptide transcript. A range of different RNAi technologies known in the art may be used to alter the activity of the FSL polypeptide. Altered FSL polypeptide activity may be determined relative to a level of activity of FSL polypeptide in a corresponding wildtype plant or part thereof in which no modification to the FSL locus sequence or expression product has taken place.

### RNA Interference

RNA interference (RNAi) is particularly useful for specifically reducing the expression of a gene, which results in reduced production of a particular protein if the gene encodes a protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated such as, for example, a FSL locus. The DNA therefore comprises both sense and antisense sequences that, when transcribed into RNA, can hybridize to form the double stranded RNA region. In one embodiment of the disclosure, the sense and antisense sequences are separated by a spacer region that comprises an intron which, when transcribed into RNA, is spliced out. This arrangement has been shown to result in a higher efficiency of gene silencing (Smith et al., 2000). The double stranded region may comprise one or two RNA molecules, transcribed from either one DNA region or two. The presence of the double stranded molecule is thought to trigger a response from an endogenous system that destroys both the double stranded RNA and also the homologous RNA transcript from the target gene, efficiently reducing or eliminating the activity of the target gene.

The length of the sense and antisense sequences that hybridize should each be at least 19 contiguous nucleotides, preferably at least 50 contiguous nucleotides, more preferably at least 100 or at least 200 contiguous nucleotides. Generally, a sequence of 100-1000 nucleotides corresponding to a region of the target gene mRNA is used. The full-length sequence corresponding to the entire gene transcript may be used. The degree of identity of the sense sequence to the targeted transcript (and therefore also the identity of the antisense sequence to the complement of the target transcript) should be at least 85%, at least 90%, or 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (“siRNA”) molecules comprise a nucleotide sequence that is identical to about 19-25 contiguous nucleotides of the target mRNA. Preferably, the siRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the organism in which it is to be introduced, for example, as determined by standard BLAST search.

microRNA

MicroRNAs (abbreviated miRNAs) are generally 19-25 nucleotides (commonly about 20-24 nucleotides in plants) non-coding RNA molecules that are derived from larger precursors that form imperfect stem-loop structures. miRNAs bind to complementary sequences on target messenger RNA transcripts (mRNAs), usually resulting in translational repression or target degradation and gene silencing. Artificial miRNAs (amiRNAs) can be designed based on natural miRNAs for reducing the expression of any gene of interest, as well known in the art.

In plant cells, miRNA precursor molecules are believed to be largely processed in the nucleus. The pri-miRNA (containing one or more local double-stranded or “hairpin” regions as well as the usual 5′ “cap” and polyadenylated tail of an mRNA) is processed to a shorter miRNA precursor molecule that also includes a stem-loop or fold-back structure and is termed the “pre-miRNA”. In plants, the pre-miRNAs are cleaved by distinct DICER-like (DCL) enzymes, yielding miRNA:miRNA* duplexes. Prior to transport out of the nucleus, these duplexes are methylated.

In the cytoplasm, the miRNA strand from the miRNA:miRNA duplex is selectively incorporated into an active RNA-induced silencing complex (RISC) for target recognition. The RISC-complexes contain a particular subset of Argonaute proteins that exert sequence-specific gene repression (see, for example, Millar and Waterhouse, 2005; Pasquinelli et al., 2005; Almeida and Allshire, 2005).

### Cosuppression

Genes can suppress the expression of related endogenous genes and/or transgenes already present in the genome, a phenomenon termed homology-dependent gene silencing. Most of the instances of homology-dependent gene silencing fall into two classes—those that function at the level of transcription of the transgene, and those that operate post-transcriptionally.

Post-transcriptional homology-dependent gene silencing (i.e., cosuppression) describes the loss of expression of a transgene and related endogenous or viral genes in transgenic plants. Cosuppression often, but not always, occurs when transgene transcripts are abundant, and it is generally thought to be triggered at the level of mRNA processing, localization, and/or degradation. Several models exist to explain how cosuppression works (see in Taylor, 1997).

Cosuppression involves introducing an extra copy of a gene or a fragment thereof into a plant in the sense orientation with respect to a promoter for its expression. The size of the sense fragment, its correspondence to target gene regions, and its degree of sequence identity to the target gene can be determined by those skilled in the art. In some instances, the additional copy of the gene sequence interferes with the expression of the target plant gene. Reference is made to WO 97/20936 and EP 0465572 for methods of implementing co-suppression approaches.

### Antisense Polynucleotides

The term “antisense polynucleotide” shall be taken to mean a DNA or RNA molecule that is complementary to at least a portion of a specific mRNA molecule encoding an endogenous polypeptide and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque (1995) and Senior (1998). Bourque (1995) lists a large number of examples of how antisense sequences have been utilized in plant systems as a method of gene inactivation. Bourque also states that attaining 100% inhibition of any enzyme activity may not be necessary as partial inhibition will more than likely result in measurable change in the system. Senior (1998) states that antisense methods are now a very well established technique for manipulating gene expression.

In one embodiment, the antisense polynucleotide hybridises under physiological conditions, that is, the antisense polynucleotide (which is fully or partially single stranded) is at least capable of forming a double stranded polynucleotide with mRNA encoding an endogenous polypeptide, for example, a FSL polypeptide mRNA (e.g., corresponding to an ORF sequence set forth in SEQ ID NO: 5-7 or a sequence having a percent level of identity thereto as described herein), a GAI1 protein mRNA (e.g., corresponding to a sequence set forth in SEQ ID NO: 8 or 9 or a sequence having a percent level of identity thereto as described herein) and/or a VvMADS5 protein mRNA (e.g., corresponding to a sequence set forth in SEQ ID NO: 10 or 11 or a sequence having a percent level of identity thereto as described herein), under normal conditions in a cell.

Antisense molecules may include sequences that correspond to the structural genes or for sequences that effect control over the gene expression or splicing event. For example, the antisense sequence may correspond to the targeted coding region of endogenous gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 750 nucleotides. The full-length sequence complementary to the entire gene transcript may be used. The length is most preferably 100-750 nucleotides. The degree of identity of the antisense sequence to the targeted transcript should be at least 90% and more preferably 95-100%. The antisense RNA molecule may of course comprise unrelated sequences which may function to stabilize the molecule.

### Recombinant Vectors

One embodiment of the present disclosure includes a recombinant vector, which comprises at least one polynucleotide defined herein and is capable of delivering the polynucleotide into a host cell. Recombinant vectors include expression vectors. Recombinant vectors contain heterologous polynucleotide sequences, that is, polynucleotide sequences that are not naturally found adjacent to a polynucleotide defined herein, that preferably, are derived from a different species. The vector can be either RNA or DNA, and typically is a viral vector, derived from a virus, or a plasmid. Plasmid vectors typically include additional nucleic acid sequences that provide for easy selection, amplification, and transformation of the expression cassette in prokaryotic cells, e.g., pUC-derived vectors, pGEM-derived vectors or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

As used herein, an “expression vector” is a DNA vector that is capable of transforming a host cell and of effecting expression of one or more specified polynucleotides. Expression vectors of the present disclosure contain regulatory sequences such as transcription control sequences, translation control sequences, origins of replication, and other regulatory sequences that are compatible with the host cell and that control the expression of polynucleotides of the present disclosure. In particular, expression vectors of the present disclosure include transcription control sequences. Transcription control sequences are sequences which control the initiation, elongation, and termination of transcription. Particularly important transcription control sequences are those which control transcription initiation such as promoter, enhancer, operator and repressor sequences. The choice of the regulatory sequences used depends on the target organism such as a plant and/or target organ or tissue of interest. Such regulatory sequences may be obtained from any eukaryotic organism such as plants or plant viruses, or may be chemically synthesized. A number of vectors suitable for stable transfection of plant cells or for the establishment of transgenic plants have been described in for example, Pouwels et al., Cloning Vectors: A Laboratory Manual, 1985, supp. 1987, Weissbach and Weissbach, Methods for Plant Molecular Biology, Academic Press, 1989, and Gelvin et al., Plant Molecular Biology Manual, Kluwer Academic Publishers, 1990. Typically, plant expression vectors include for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, a transcription termination site, and/or a polyadenylation signal.

A number of constitutive promoters that are active in plant cells have been described. Suitable promoters for constitutive expression in plants are known in the art and described previously herein.

For the purpose of expression in source tissues of the plant such as, for example, in flowers and reproductive parts thereof, buds, fruit, root or stem, it may be preferred that the promoters utilized in the present disclosure have relatively high expression in these specific tissues. For this purpose, one may choose from a number of promoters for genes with tissue- or cell-specific, or -enhanced expression. Examples of such promoters are reported in the literature and will be known to a person skilled in the art.

“Operably linked” as used herein, refers to a functional relationship between two or more nucleic acid (e.g., DNA) segments. Typically, it refers to the functional relationship of a transcriptional regulatory element (promoter) to a transcribed sequence. For example, a promoter is operably linked to a coding sequence of a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements such as enhancers need not be physically contiguous or located in close proximity to the coding sequences whose transcription they enhance.

When there are multiple promoters present, each promoter may independently be the same or different.

Recombinant vectors may also contain one or more signal peptide sequences to enable an expressed polypeptide defined herein to be retained in the endoplasmic reticulum (ER) in the cell, or transfer into a plastid, and/or contain fusion sequences which lead to the expression of nucleic acid molecules as fusion proteins. Examples of suitable signal segments include any signal segment capable of directing the secretion or localisation of a polypeptide defined herein.

To facilitate identification of transformants, the recombinant vector desirably comprises a selectable or screenable marker gene. By “marker gene” is meant a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., a herbicide, antibiotic). A screenable marker gene (or reporter gene) confers a trait that one can identify through observation or testing, that is, by “screening” (e.g., β-glucuronidase, luciferase, GFP or other enzyme activity not present in untransformed cells). Exemplary selectable markers for selection of plant transformants include, but are not limited to, a hyg gene which encodes hygromycin B resistance; a neomycin phosphotransferase (nptII) gene conferring resistance to kanamycin, paromomycin; a glutathione-S-transferase gene from rat liver conferring resistance to glutathione derived herbicides as for example, described in EP 256223; a glutamine synthetase gene conferring, upon overexpression, resistance to glutamine synthetase inhibitors such as phosphinothricin as for example, described in WO 87/05327; an acetyltransferase gene from Streptomyces viridochromogenes conferring resistance to the selective agent phosphinothricin as for example, described in EP 275957; a gene encoding a 5-enolshikimate-3-phosphate synthase (EPSPS) conferring tolerance to N-phosphonomethylglycine as for example, described by Hinchee et al. (1988); a bar gene conferring resistance against bialaphos as for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS) which confers resistance to imidazolinone, sulfonylurea, or other ALS-inhibiting chemicals (EP 154,204); a mutated anthranilate synthase gene that confers resistance to 5-methyl tryptophan; or a dalapon dehalogenase gene that confers resistance to the herbicide.

The 5′ non-translated leader sequence can be derived from the promoter selected to express the polynucleotide of the present disclosure, or may be heterologous with respect to the coding region of the enzyme to be produced, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present disclosure is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. Nos. 5,362,865 and 5,859,347), and the TMV omega element.

The termination of transcription is accomplished by a 3′ non-translated DNA sequence operably linked in the expression vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Recombinant DNA technologies can be used to improve expression of a transformed polynucleotide by manipulating, for example, the efficiency with which the resultant transcripts are translated by codon optimisation according to the host cell species or the deletion of sequences that destabilize transcripts, and the efficiency of post-translational modifications.

Preferably, the recombinant vector is stably incorporated into the genome of the cell such as the plant cell. Accordingly, the recombinant vector may comprise appropriate elements which allow the vector to be incorporated into the genome, or into a chromosome of the cell.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). A genetically modified plant formed using Agrobacterium transformation methods typically contains a single genetic locus on one chromosome. Such genetically modified plants can be referred to as being hemizygous for the added gene or gene variant. More preferred is a genetically modified plant that is homozygous for the added gene or gene variant; i.e., a genetically modified plant that contains two added genes, one gene at the same locus on each chromosome of a chromosome pair. A homozygous genetically modified plant can be obtained by sexually mating (selfing) an independent segregant genetically modified plant that contains a single added gene o gene variant, germinating some of the seed produced and analyzing the resulting plants for the gene of interest.

It is also to be understood that two different genetically modified plants can also be mated/crossed to produce offspring that contain two independently segregating introduced genes or gene variants. Selfing of appropriate progeny can produce plants that are homozygous for both introduced genes or gene variants. Back-crossing to a parental plant and out-crossing with a further plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of polynucleotides such as DNA into plants by direct transfer into pollen, by direct injection of polynucleotides such as DNA into reproductive organs of a plant, or by direct injection of polynucleotides such as DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

To confirm the presence of the introduced genetic material in cells and plants, a polymerase chain reaction (PCR) amplification or Southern blot analysis can be performed using methods known to those skilled in the art. Expression products of the introduced gene or gene variant can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Cells

The present disclosure also provides a recombinant cell comprising a host cell transformed with one or more recombinant molecules as defined herein, or progeny cells thereof. Transformation of a nucleic acid molecule into a cell can be accomplished by any method by which a nucleic acid molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed nucleic acid molecules can remain extrachromosomal or can integrate into one or more sites within a chromosome of the transformed (i.e., recombinant) cell in such a manner that their ability to be expressed is retained. Preferred host cells are plant cells, more preferably cells area grapevine cell.

The present disclosure also provide a plant cell which has been isolated from a plant or plant part of the disclosure. For example, a cell isolated from a plant or plant part having an altered level of FSL polypeptide activity as described herein. For example, a cell isolated from a plant or plant part which produces seedless fruit as described herein. In some examples, the cell is cultured.

### Plants and Plant Parts

The term “plant” when used as a noun refers to whole plants, whilst the term “plant part” or “part thereof” (in the context of a plant) refers to a plant cell and progeny of same, a plurality of plant cells, a structure that is present at any stage of a plant's development, or a plant tissue. Such structures include, but are not limited to, leaves, stems, cutting and scion, flowers, fruits, nuts, roots, seed, seed coat, embryos. The term “plant tissue” includes differentiated and undifferentiated tissues of plants including those present in leaves, stems, flowers, fruits, nuts, roots, seed, for example, embryonic tissue, endosperm, dermal tissue (e.g., epidermis, periderm), vascular tissue (e.g., xylem, phloem), or ground tissue (comprising parenchyma, collenchyma, and/or sclerenchyma cells), as well as cells in culture (e.g., single cells, protoplasts, callus, embryos, etc.). Plant tissue may be in planta, in organ culture, tissue culture, or cell culture.

As used herein, “progeny” means the immediate and all subsequent generations of offspring produced from a parent, for example a second, third or later generation offspring.

As used herein, the term “plant” includes all species of flowering plants i.e., angiosperm. In one example, the plant as described herein is a dioecious plant. In another example, the plant as described herein is a hermaphroditic plant, The plant is preferably a fruit producing plant. For example, the plant may be a berry producing plant, a hesperidia producing plant, a drupe producing plant, a pome producing plant, or a pepo producing plant. Exemplary fruit producing plants within each of those broad fruit categories are known in the art and contemplated herein.

Plants contemplated for use in the practice of the present disclosure include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers).

In one particular example, the plant is a berry producing plant. For example, the plant may be a Vitis sp. e.g., a Vitis species selected from the group consisting of: Vitis vinifera, Vitis lambrusca, Vitis rotundifolia, Vitis aestivalis, Vitus riperia and hybrids thereof. In one example, the Vitis sp produces table grapes. In another example, the Vitis sp. produces wine grapes. Vitis rotundifolia is also known as Muscadinia rotundifolia and includes other Muscadinia species.

### Method of Producing Plants and Plant Parts

There are many techniques known in the art which can be used to produce plants with an altered level of FSL polypeptide activity as described herein, including plants and plant parts that produce seedless fruit as described herein, including, but not limited to, TILLING, zinc finger nuclease (ZFN), TAL effector nuclease (TALEN), and Clustered Regularly Interspaced Short Palindromic Repeats (CRISPR).

### Tilling

Plants of the disclosure can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling and 96 lanes per assay, this combination allows up to a million base pairs of genomic DNA to be screened per single assay, making TILLING a high-throughput technique.

TILLING is further described in Slade and Knauf (2005) and Henikoff et al., (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

In ecotilling plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. PCR products used for screening can be subjected to DNA sequencing.

### Genome Editing Using Site-Specific Nucleases

Genome editing uses engineered nucleases composed of sequence specific DNA binding domains fused to a non-specific DNA cleavage module. These chimeric nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs) and transcription activator-like (TAL) effector nucleases (TALEN).

Typically nuclease encoding genes are delivered into cells by plasmid DNA, viral vectors or in vitro transcribed mRNA. The use of fluorescent surrogate reporter vectors also allows for enrichment of ZFN- and TALEN-modified cells. As an alternative to ZFN gene-delivery systems, cells can be contacted with purified ZFN proteins which are capable of crossing cell membranes and inducing endogenous gene disruption.

Complex genomes often contain multiple copies of sequences that are identical or highly homologous to the intended DNA target, potentially leading to off-target activity and cellular toxicity. To address this, structure (Miller et al., 2007; Szczepek et al., 2007) and selection based (Doyon et al., 2011; Guo et al., 2010) approaches can be used to generate improved ZFN and TALEN heterodimers with optimized cleavage specificity and reduced toxicity.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell or organism. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Sp1. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques. (see, for example, Bibikova et al., 2002).

The ZFN DNA-cleavage domain is derived from a class of non-specific DNA cleavage domains, for example the DNA-cleavage domain of a Type II restriction enzyme such as FokI (Kim et al., 1996). Other useful endonucleases may include, for example, HhaI, HindIII, Nod, BbvCI, EcoRI, BgII, and AlwI.

A linker, if present, between the cleavage and recognition domains of the ZFN comprises a sequence of amino acid residues selected so that the resulting linker is flexible. Or, for maximum target site specificity, linkerless constructs are made. A linkerless construct has a strong preference for binding to and then cleaving between recognition sites that are 6 bp apart. However, with linker lengths of between 0 and 18 amino acids in length, ZFN-mediated cleavage occurs between recognition sites that are between 5 and 35 bp apart. For a given linker length, there will be a limit to the distance between recognition sites that is consistent with both binding and dimerization. (Bibikova et al., 2001). In a preferred embodiment, there is no linker between the cleavage and recognition domains, and the target locus comprises two nine nucleotide recognition sites in inverted orientation with respect to one another, separated by a six nucleotide spacer.

In order to target genetic recombination or mutation according to a preferred embodiment of the present invention, two 9 bp zinc finger DNA recognition sequences must be identified in the host DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

ZFN activity can be improved through the use of transient hypothermic culture conditions to increase nuclease expression levels (Doyon et al., 2010) and co-delivery of site-specific nucleases with DNA end-processing enzymes (Certo et al., 2012). The specificity of ZFN-mediated genome editing can be improved by use of zinc finger nickases (ZFNickases) which stimulate HDR without activation the error-prone NHE-J repair pathway (Kim et al., 2012; Wang et al., 2012; Ramirez et al., 2012; McConnell Smith et al., 2009).

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BgII, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations. CRISPR systems rely on CRISPR RNA (crRNA) and transactivating chimeric RNA (tracrRNA) for sequence-specific silencing of invading foreign DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

The CRISPR system can be portable to plant cells by co-delivery of plasmids expressing the Cas endonuclease and the necessary crRNA components as described in the art and herein. The Cas endonuclease may be converted into a nickase to provide additional control over the mechanism of DNA repair (Cong et al., 2013).

CRISPR loci are a distinct class of interspersed short sequence repeats (SSRs) that were first recognized in E. coli (Ishino et al., 1987; Nakata et al., 1989). Similar interspersed SSRs have, been identified in Haloferax mediterranei, Streptococcus pyogenes, Anabaena, and Mycobacterium tuberculosis (Groenen et al., 1993; Hoe et al., 1999; Masepohl et al., 1996; Mojica et al., 1995).

The common structural characteristics of CRISPR loci are described in Jansen et al., (2002) as (i) the presence of multiple short direct repeats, which show no or very little sequence variation within a given locus; (ii) the presence of non-repetitive spacer sequences between the repeats of similar size; (iii) the presence of a common leader sequence of a few hundred basepairs in most species harbouring multiple CRISPR loci; (iv) the absence of long open reading frames within the locus; and (v) the presence of one or more cas genes.

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

As used herein, the term “cas gene” refers to one or more cas genes that are generally coupled associated or close to or in the vicinity of flanking CRISPR loci. A comprehensive review of the Cas protein family is presented in Haft et al. (2005). CRISPR-Cas systems most frequently adopted in eukaryotic work use a Cas9 effector protein typically using the RNA-guided Streptococcus pyogenes Cas9 or an optimised sequence variant in multiple plant species (Luo et al., 2016). Cas as used herein includes Cas9, Cas12 enzymes (e.g Cas12a, Cas12b, Cas12f, Cpf1, C2cl, C2c3) and other CRISPR-Cas systems such as the RNA-guided Cas13 RNAses.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S. pyogenes Cas9 nuclease with a D10A mutation or H840A mutation.

### Genome Base Editing or Modification

Base editors have been created by fusing a deaminase with a Cas9 domain (WO 2018/086623). By fusing the deaminase can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Conventional Plant Breeding Methods

In addition to the methods described above, plants of the present disclosure may also be produced using conventional plant breeding techniques known in the art. Such methods generally involve crossing parental lines having known polynucleotides or gene, including specific variants same, which confer particular traits, and screening the progeny produced by the crosses to identify progeny having particular combinations of polynucleotides or genes of interest and/or exhibiting particular phenotype(s) of interest. Screening of progeny may be on the basis of phenotype and/or based on molecular characterisation using molecular techniques known in the art. In some examples, conventional breeding methods involve subsequent backcrosses to parental lines in order to achieve a particular genotypic state at one or more polynucleotides or genes. Conventional plant breeding techniques are well known in the art and contemplated herein to produce plants that produce flowers of known sex and/or to produce plants which produce seedless fruit, as described herein.

### Fruit and Products Thereof

Also provided herein is fruit produced from a plant described herein. The fruit may be seedless. Preferably the fruit are stenospermocarpic seedless. More preferably, the fruit are parthenocarpic seedless. In accordance with an example in which the plant from which the fruit is grown is a Vitis sp., the fruit will be grapes e.g., seedless grapes. In one example the grapes are seedless table grapes.

Fruit of the present disclosure can be produced by growing a plant as described herein under conditions and for a period sufficient for the plant to flower and produce fruit. In some examples, the fruit may be harvested from the plant. However, in other examples it may be desired to leave the fruit on the plant (e.g., for nursery sale). In some examples, the method further comprises processing the fruit. For example, processing the fruit may comprise packaging the fruit and/or producing one or more product (e.g., one or more food or beverage products or ingredients) from the fruit.

The present disclosure also provides a product produced from a plant as described herein or fruit thereof. In one example, the product is a food product, food ingredient, beverage product or beverage ingredient. The food product may be selected from the group consisting of table grapes, jam, marmalade, jelly, sultanas, and raisins, for example. The food ingredient may be vincotto, verjuice, vinegar or grape must syrup (mosto cotto), for example. The beverage product may be is wine, grappa, brandy or grape juice. For example. The beverage ingredient may be wine grapes, table grapes or juice therefrom, for example.

### EXAMPLES

### Example 1: Material and Methods

### Microvine Plant Lines

Microvine plants were grown in glasshouses or growth rooms under 16-h days at 25-30° C. and 20-25° C. nights at the CSIRO Urrbrae site, Adelaide, Australia. Plants were maintained in pots, watered daily and given slow release fertiliser at regular intervals. Microvine genotypes studied had either male flowers (FSL/fsl), female flowers (fsl/fsl) or hermaphroditic flowers (FSL/FSL or FSL/fsl). A number of microvine lines with different flower types were studied and examples include 03C003V0060 (L1 Pinot Meunier progeny×Richter 110) (male flowers, M/f), 04C023V0003 (female flowers, f/f), 04C023V0006 (hermaphrodite flowers, H/H). Microvine lines with male flowers were obtained by crossing the grapevine rootstock Richter 110 (M/f) with the female microvine line 00C001V0008 (f/f).

Phenotyping of flower sex was performed by morphological scoring using the OIV descriptors No 151 (http://www.oiv.int/).

Genomic DNA was extracted from microvine leaves using the DNAeasy Plant Mini Kit (Qiagen 69106).

### Cloning of Male, Hermaphrodite and Female Alleles

To obtain coding DNA sequence and translated protein sequence, total RNA was extracted using the Spectrum Plant Total RNA kit Cat #STRN250 (Sigma) as per the manufacturer's instructions from flower stages 1-2 of the modified E-L system A description of the modified E-L system can be found in the paper by B. G. Coombe ‘Adoption of a system for identifying grapevine growth stages’ (1995) Aust. J. Grape and Wine Res. 1:104-110. Total RNA was extracted from the FSL gene edited plant (FSL knockout), the male plant 03C003V0016 (progeny from L1 Pinot Meunier self-cross progeny×Richter 110), the female plant 04C023V0003 (progeny from Grenache×L1 Pinot Meunier) and the homozygous hermaphrodite 04C023V0006 (progeny from Grenache×L1 Pinot Meunier). First strand cDNA was generated using the Superscript IV First Stand Synthesis System Cat #18091050 (Invitrogen) following the manufacturer's instructions using the oligo dT B26 5′-GACTCGAGTCGACATCGATTTTTTTTTTTTTTTTT-3′ (SEQ ID NO: 12). The coding sequences amplified from the cDNA using standard PCR techniques using MyTaq™ HS Red Mix from Bioline catalogue #BIO-25047 following manufacturer's instructions for a 20 μl reaction volume and 2 μl of template. PCR reaction conditions were 95° C. for 3 minutes for an initial denaturation and then 35 cycles of 95° C. 30 seconds, 58° C. 30 seconds, 72° C. 1 minute and 40 seconds. There was a final extension incubation at 72° C. for 10 minutes. The primers used for amplification were B26 and CSFS1_CDS_F1 5′-CAG TGC CAG TTT TGC AGG C-3′ (SEQ ID NO: 13) with primers designed from the Cabernet Sauvignon cDNA H sequence in Example 2. PCR products were cloned for sequencing using the Qiagen PCR Cloning Kit Catalogue #231124 as per the manufacturer's instructions.

### FSL Expression in Flower Stage 1-2 in the Hermaphrodite and Female Lines

Gene expression of FSL was measured from 1^{st} strand cDNA using RT-qPCR. The 1^{st} strand cDNA and gene specific primers were designed to the 3 prime region for each allele. Primers used were:

FSL_RT_F1 5′-ACGCCGGTGGAATAAACAGG-3′ (SEQ ID NO: 14); and

FSL_RT_R1 5′-TCT CCT TTC TCC ATC CCT AAT TGA-3′ (SEQ ID NO: 15).

The LightCycler 480 SYBR Green 1 Master 2×concentration cat #04 887 352 001 (Roche) was used at a 1×concentration along in the RT-qPCR assay along with gene specific primers 10 pMol per reaction (1 μl of a 10 μM stock) and 3 μl of first strand cDNA in a 15 μl reaction volume. PCR amplification was done in a Rotor-Gene RG-3000 (Corbett research), 95° C. for 10 minutes then 50 cycles of 95° C. 20 seconds, 58° C. 20 seconds, 72° C. 20 seconds and then a final step of 72° C. for 5 minutes. Standard curves, melt curves and Ct values were generated for each gene and for each cDNA sample using Rotor-Gene 3000 software (Corbett Research). Ct values were normalized using the reference gene, Ubiquitin to determine relative gene expression level in each sample using the relative quantification comparative method described in the Qiagen handout “Critical Factors For Successful Real-Time PCR” (https://www.gene-quantification.de/qiagen-qpcr-sample-assay-tech-guide-2010.pdf).

### Pollen Germination Assay

Anthers from flowers were collected on single cavity slides and allowed to dry for 5 minutes in order to release the pollen. The pollen was collected in a germinating solution (0.5M sucrose, boric acid 100 mg/mL and calcium nitrate 300 mg/L pH 5.4) and incubated overnight on an inverted slide in a humidified container at 28 degrees Celsius in the dark. Pollen germination was assessed at 20× magnification by differential interferance contrast (DIC) brightfield microscopy.

Genetic transformation of the homozygous hermaphrodite (H/H) 04C023V0006 with the FSL Gene Editing Vector

The binary vector pCLB1301NH containing the gene editing cassette cas9-sgRNA cassette was inserted into the homozygous hermaphrodite 04C023V0006 by Agrobacterium-mediated transformation. The full method is described in locco et al., (2001) and transgenic plants regenerated using the method described in Chaib, J et. al., (2010) Plant Journal, 62(6):1083-1092. The gene editing vector design is described in more detail in Example 6.

### Grapevine Inbred Line Development and Breeding

Production of homozygous lines differing in both stature and flower sex are desirable to improve future breeding efficiency of microvines and vines of normal stature. Identification of the FSL gene and sequences for selection will allow breeders to screen out female plants at seedling stage. To achieve this successive inbreeding by self-fertilization (selfing) was conducted while maintaining a heterozygous state at the two loci for stature (VvGAI1/Vvgai1) and flower sex (FSL/fsl). The original L1 Pinot Meunier mutant microvine was used as a female parent and flowers were emasculated to receive pollen from Cabernet Sauvignon or Riesling or other desired cross, as male parents. Progeny populations were germinated from collected seeds after soaking for 12 hours in fresh 0.5M H₂O₂, rinsed in sterile water and transferred to sterile 2.6 mM GA (gibberellic acid) for overnight incubation before rinsing again in sterile water and sealed moist. The seeds were stored at 4° C. for approximately 3 weeks, seeds were scarified, incubated again in GA for 18 h and transferred to a Petri dish lined with sterile filter paper. Seeds were kept at 25 C under lights and after germination were transferred to pots and kept either in a growth chamber or glasshouse with 16 h days at 25-30° C. and 20-25° C. nights. The segregation of the stature allele resulted in 50% progeny possessing dwarf stature. The FSL locus followed the expected segregation ratio of 1:1 from a FSL/fsl×fsl/fsl cross. Microvine individuals were isolated for each population and were grown in a glasshouse. The vines can be grown in high density 34 microvines per m².

### Example 2: Cloning the Hermaphroditic Sex Locus by Gene Mapping and Cloning and Sequencing the Male, Female and Hermaphrodite Alleles

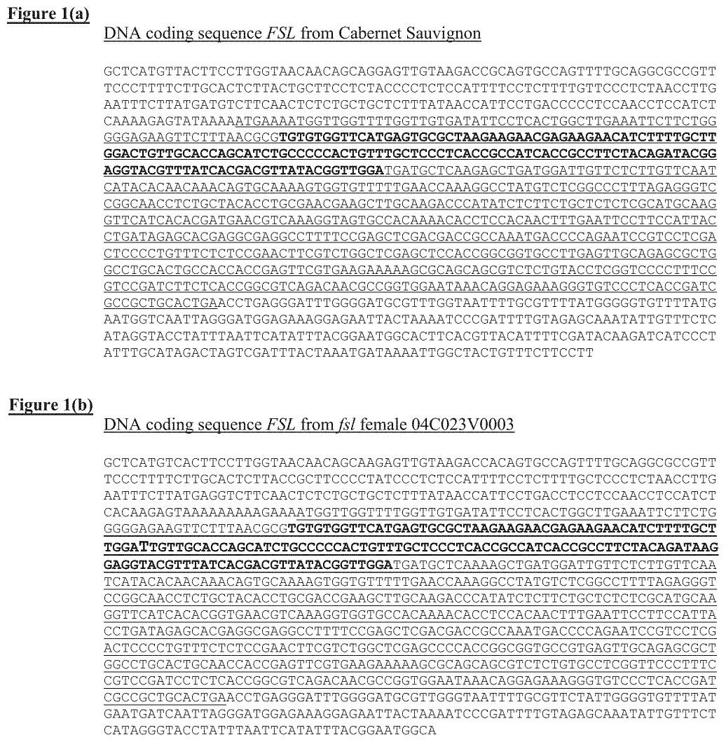

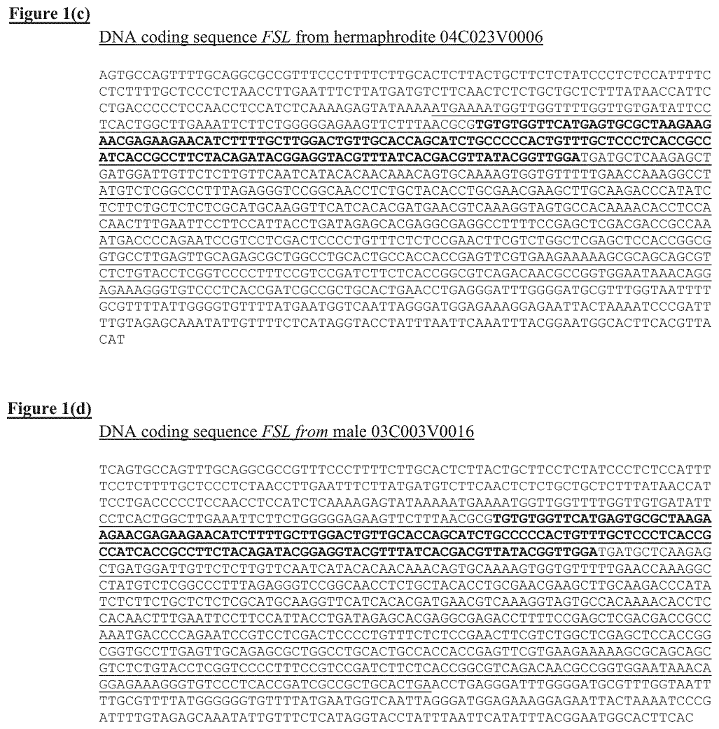

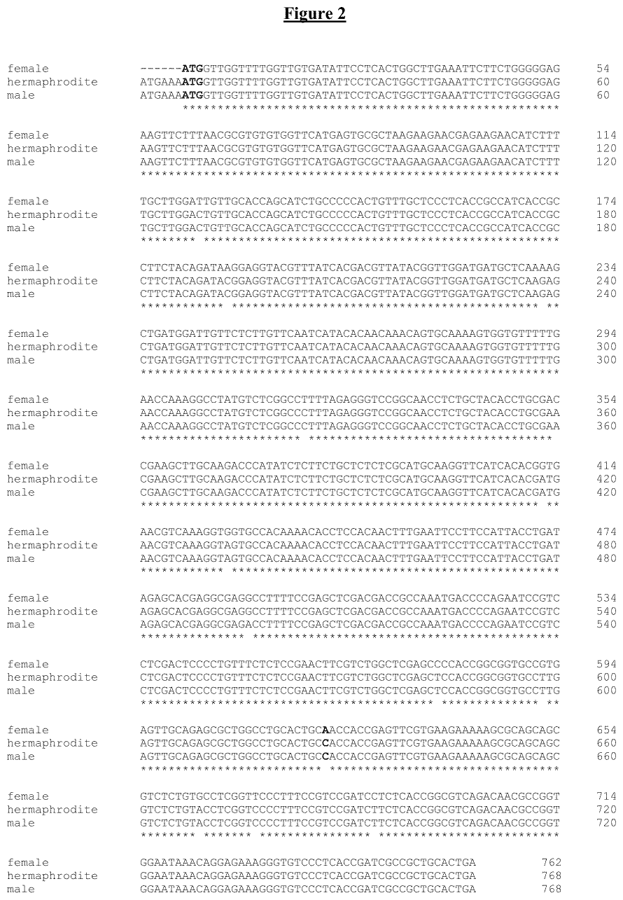

By genetic mapping, the inventors have identified a gene thought to be responsible for flower sex in grapevines. The inventors have name it the Flower Sex (FSL) gene. Sequencing of this locus identified single nucleotide polymorphisms (SNPs) between the male (M), female (f) and hermaphroditic (H) alleles of FSL. A SNP marker from this gene has been used for genotyping plants for the H and f alleles and there is 100% match between the genotype and the phenotype. A full length H cDNA from Cabernet Sauvignon was sequenced from a flower cDNA library produced at CSIRO using standard molecular methods. The cDNA library was made from immature inflorescences at stage 12 of the modified E-L system. Tissue was collected from field grown plants. A description of the modified E-L system can be found in the paper by B. G. Coombe (1995) Aust. J. Grape and Wine Res., 1:104-110. The cDNA for FSL from Cabernet Sauvignon is set forth in FIG. 1A and SEQ ID NO: 1.

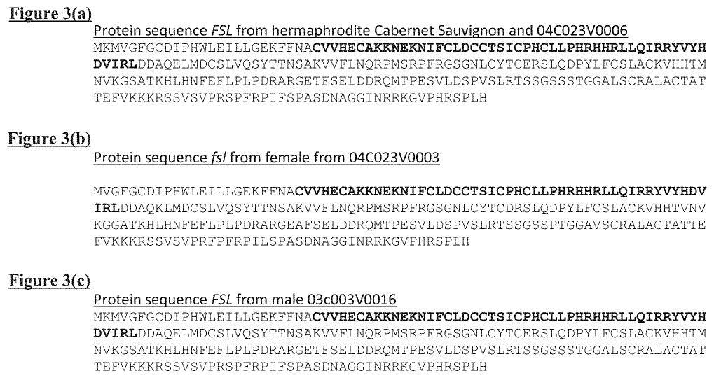

Using cloning methods described in Example 1, to isolate FSL from the male, female and hermaphrodite from the genotypes 03C003V0016 and 04C023V003 and 04C023V0006 were isolated. These are shown in FIGS. 1B-D, respectively, and set forth in SEQ ID NOs: 2-4, respectively. An alignment of the open reading frames (ORFs) for the female, hermaphrodite and male alleles of the FSL locus (SEQ ID NOs: 5-7 respectively) is provided in FIG. 2.

### Example 3: Protein Sequences and Blast Analysis

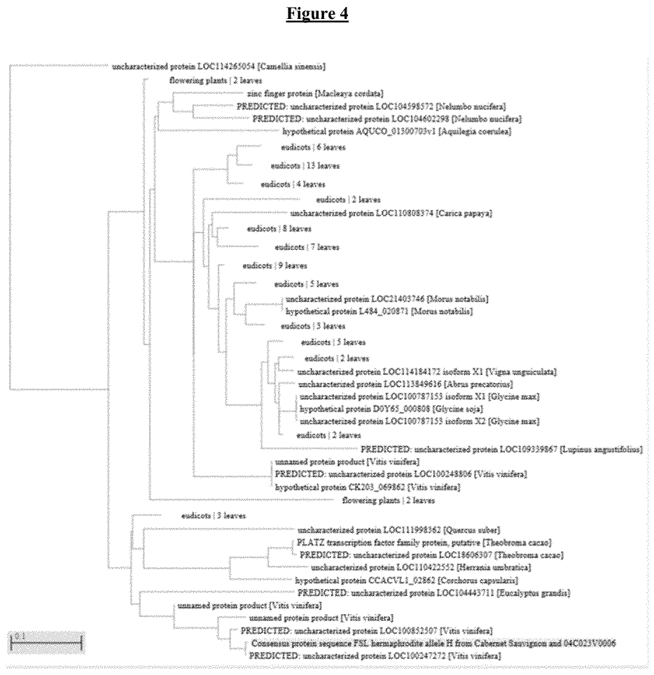

Protein sequences were derived from the cDNA sequences and blast analysis was performed for both the protein and cDNA sequences. The protein sequences are set forth in FIG. 3A-C and SEQ ID NOs: 5-7. These alignments predicted the presence of a PLATZ (plant AT-rich sequence- and zinc-binding) domain (Nagano et al., (2001) Nucleic Acids Res. 29(20):4097-4105).

The predicted region for the PLATZ domain (Zinc finger Box) is highlighted in yellow for both cDNA sequences (FIGS. 1A-D) and amino acid sequences (FIGS. 3A-C). The online tool PROSITE (Sigrist, C. J. A., (2009) Nucleic Acids Research, 38:161-166)) was used to identify the region in each case. As is apparent from the sequences, there is no difference in the amino acid sequence between the various alleles, however there is a C to T substitution within the cDNA sequence of the female allele (bold type) which gives rise to a sense mutation GAC->GAT which both code for aspartic acid (see FIGS. 1B-D).

A phylogenetic tree was also created for the hermaphrodite protein sequence (FIG. 3). Most of the hits were to uncharacterised sequences with no known function. The protein sequence alignments and hits obtained for the phylogenetic tree support the conclusion that the FSL gene contains a PLATZ domain and that it is likely to be a transcription factor. PLATZ super family of transcription factors have been found to exist only in plants and have not so far been identified as having an involvement in flower sex determination. In fact, the precise function of PLATZ proteins in plants remains poorly understood and there are indications that they may function in response to stress (So et al, (2015) POJ, 8(6):479-484.

The amino acid sequence of the protein is similar to those of other uncharacterized proteins predicted from the genome sequences of higher plants. However, no orthologous sequences have been found outside the plant kingdom. Multiple alignments among these orthologous proteins show that several cysteine and histidine residues are invariant, suggesting that these proteins are a novel class of zinc-dependent DNA-binding proteins (Nagano et al. 2001).

### Example 4: Expression of Flower Sex (Fsl) Gene in Grapevine Flowers

To better understand the function of FSL in specifying flower sex, mRNA in situ hybridization was performed to identify the floral organs and tissues where this gene is transcribed. A digoxigenin labeled 711 bp probe was synthesized from the FSL gene (54-765 bp of the sequence set forth in SEQ ID NO: 4). Blast analysis of the 711 bp probe indicated that this probe is specific for hybridization with FSL transcripts. Flowers at a young immature stage (flowers not separated from each other) were used to determine the expression pattern of FSL. The in situ hybridization was performed according to the methodologies described in Jackson, D. P. (1992) In-situ hybridisation in plants. In: Molecular Plant Pathology: A Practical Approach. Practical Approach Series, 1 (85). Oxford University Press and https://www.its.caltech.edu/˜plantlab/protocols/insitu.pdf.

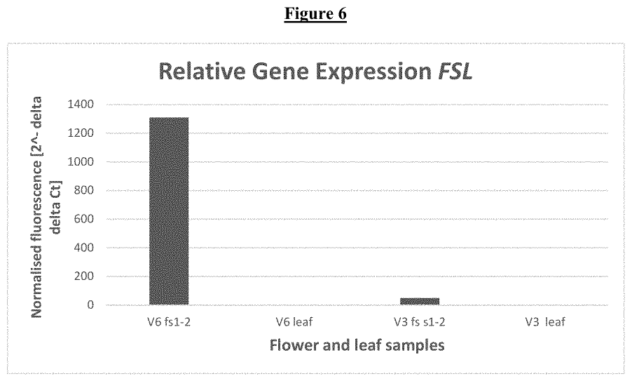

Results from the mRNA in situ hybridisation showed that FSL was expressed in the filament and anthers of the stamens, as well as the ovule of male flowers (FIGS. 5A and B). In hermaphrodite flowers, expression was detected in the filaments of the stamen and ovule (FIGS. 5C and D). Compared to male flowers, FSL expression appeared to be reduced in the anthers of hermaphrodite flowers. In male and hermaphrodite flowers, FSL expression was not detected in the perianth organs (FIG. 5A-D). As female flowers are expected to display non-functional FSL phenotype, little or no expression was observed in the anthers and filaments of the stamens (FIG. 5E-F). Interestingly, FSL appears to be expressed in the ovule (FIG. 5E). Taken together, these results show that FSL is primarily expressed in the stamens of male and hermaphrodite flowers and expression of this gene is absent in female flowers in which the male reproductive organs are non-functional.

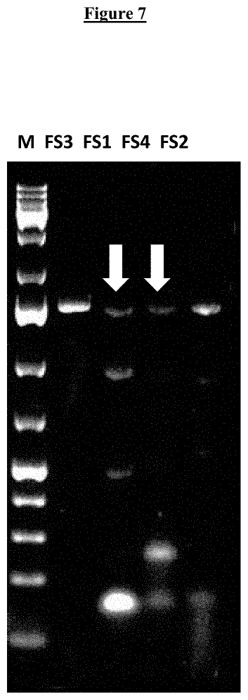

Real time PCR analyses was also performed on cDNAs obtained from leaves and early developing flowers stages 1-2 when the flowers are still compact and tightly closed to determine the expression pattern of FSL using the method as described in Example 1. As is apparent from FIG. 6, expression of FSL is highest in the V6 homozygous hermaphrodite (H/H) and is very low in the V3 female (f/f) which supports the finding that FSL is involved in flower sex determination through normal stamen development. The results of this experiment indicate that the f allele was expressed in the flowers 27 times less than the H allele. This data indicates that FSL is down regulated to produce the female flower phenotype.

### Example 5: Gene Editing to Convert Grapevine Flowers from Hermaphrodite to Female

### Methods and Results

CRISPR/Cas9 mediated mutations were introduced within the putative PLATZ domain of the FSL gene with the aim of producing an FSL knockout microvine plant in order to determine gene function in flowering. The CRISPR/Cas9 vector had spCas9 followed directly by the single crRNA:tracRNA sgRNA (Jinek et al., (2012) Science, 337(6096): 816-821). The SpCas9 was codon optimized for Vitis vinifera to optimize translation efficiency.

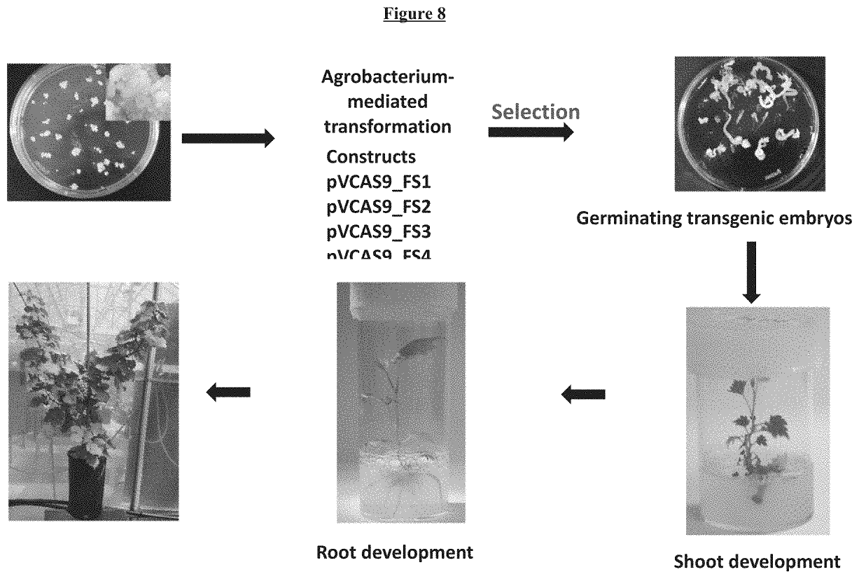

All possible 20 bp guide RNAs for FSL were identified using the online tool Benchling (https://benchling.com). Guide RNAs which were located within the putative PLATZ domain were selected and then screened for in vitro cleavage with CAS9 and the DNA template using the Guide-it sgRNA In vitro Transcription and Screening Systems catalogue #632639 (Takara Bio USA, Inc). Two guide RNAs were selected and were named sgRNAFS1 and sgRNAFS4 (FIG. 7).

The CRISPR/Cas9-sgRNA cassette was synthesized by Genscript (https://www.genscript.com/) and cloned into the binary vector pCLB1301NH for transformation using the general methodology described in Example 1. The sgRNA sequences used were as follows:

| | Guide FS1 (in antisense orientation): |
|---|---|
| (SEQ ID NO 16) | |
| | GGCGGTGAGGGAGCAAACAG |
| | Guide FS4 (in antisense orientation): |
| (SEQ ID NO 17) | |
| | AGGGGTGCACCTGTAGAAGG |
| | Guide FS2 (in antisense orientation): |
| (SEQ ID NO 18) | |
| | GTCTTGCAAGCTTCGTTCGC |
| | Guide FS3 (in sense orientation): |
| (SEQ ID NO 19) | |
| | GCAGCAGCGTCTCTGTACCT |

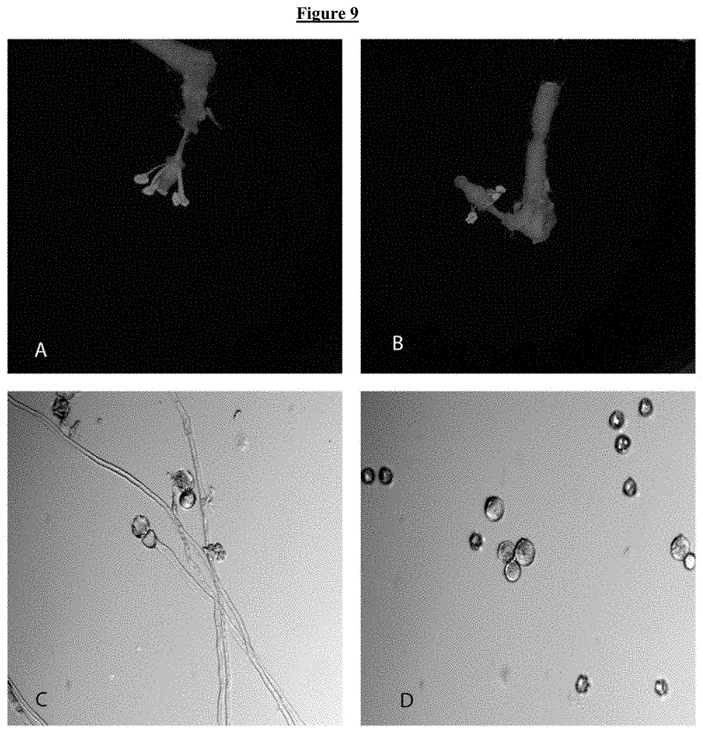

The genetic transformation of the microvines is illustrated in FIG. 8.

Resulting transgenic T0 generation plantlets were checked for gene editing by amplicon Sanger sequencing and over 62% of plants had edits in the predicted region of FSL. Some plants were analyzed further with Next Gen sequencing to determine mutation type, position and frequency. The T0 plants with high mutation frequency around the predicted location were selected for crossing for the T1 generation.

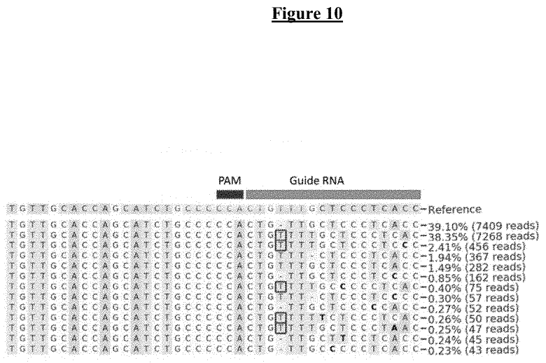

The T0 generation were also phenotyped for flower sex using the method described in Example 1. Two plants edited by sgRNAFS1 developed female flowers whereby the stamens were reflexed and the pollen was infertile as determined by a pollen germination assay described in example 1. FIG. 9 shows the flower phenotypes for the original hermaphrodite plant and the FSL knock out plant.

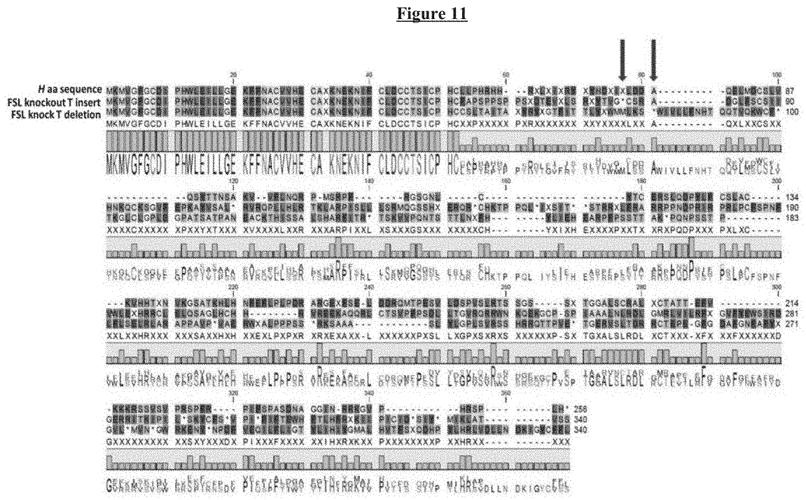

The mutation type position and frequency of the mutations in the FSL knock out plant were determined by Amplicon NextGen sequencing. The mutation frequency around the guide sequence was 98% for both leaf and flower genomic DNA samples implying that both alleles have been mutated by gene editing and that the mutation can be transmitted to the T1 generation. FIG. 10 shows the most frequent mutation type and position being either a T insertion or a T deletion at the 16^{th} base of the guide sequence. The resulting edits of a T insertion or a T deletion correspond to positions 155 and 159 relative to the sequence set forth in SEQ ID NO: 6 or 7.

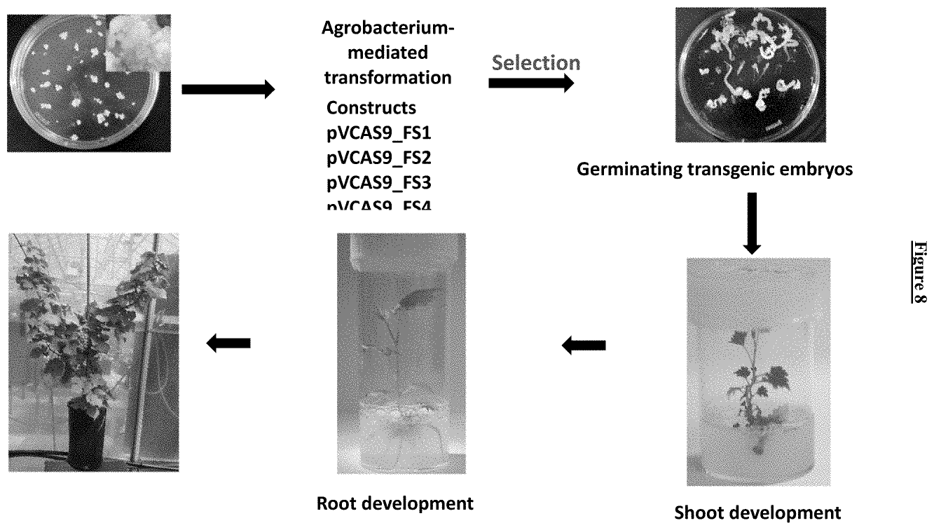

Alignment of the predicted amino acid sequences for the FSL knock out and the H allele show that the knockout produced a nonsense mutation where protein synthesis is prematurely aborted due to a stop codon (FIG. 11).

### Discussion

The conversion of hermaphrodite to female flower by gene editing of FSL within the microvine strongly supports the conclusion that FSL is involved in male organ development, corroborating the findings in Example 4. The mutations introduced by gene editing resulted in a truncated nonfunctional protein, thereby preventing the male reproductive organs to develop and giving rise to the female flower phenotype. Thus, the present disclosure provides a novel method and overall general approach for converting hermaphrodite flowering plants to female flowering plants. Such methods and approaches may be a useful in male sterility selective breeding strategies.

### Example 6: Dwarf Female Grapevines with Parthenocarpic Seedless Fruit

Building on the finding that the FSL gene is responsible for flower sex in grapevines, the inventors then developed dwarf plants that flower rapidly and produce seedless fruit and a method for producing same. Growing conditions of microvines and protocols for breeding and seed germination were described in Chaib et al, 2010. Plants were grown in a glasshouse in Waite, South Australia. This was achieved by combining two genes in a single plant: 1) the mutated gene Gibberellic Acid Insensitive gene in either the heterozygous (GAI1/gai1) or homozygous state (GAI1/GAI1) that causes a dwarf stature and rapid flowering phenotype; and 2) the female FSL locus (f/f in the homozygous state). Populations were visually phenotyped for plant stature and berry colour.

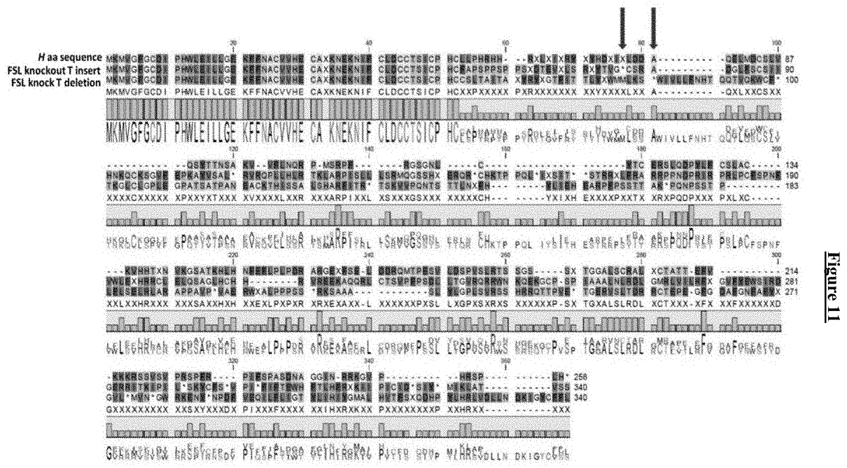

Plants having the above genetic profile were then tested by artificially pollinating some of the inflorescences of female microvines at the time of anthesis with viable pollen from a genotype such as Riesling, and leaving others unpollinated. Inflorescences were tagged with paper tags showing the name of pollen donor and date of pollination. The pollinated inflorescences were allowed to grow into bunches and fruits were harvested and sectioned to observe seed development (if any) around 3 months after pollination. Hard seeds were found in the sections of those berries that developed from pollinated flowers on the female microvine, but no seeds were found in the berries that developed from non-pollinated flowers (FIGS. 12A and B).

Normal female grapevines produce flowers which develop no fruit when unpollinated, but fruit is produced when those flowers are cross pollinated with pollen from male or hermaphroditic plants. By contrast, the female microvines developed herein have been shown to produce berries that are generally seedless unless cross pollinated. Thus, the dwarfing gene in combination with FSL f/f genotype produces seedless fruit in the absence of pollination, but produce hard viable seeds when flowers are pollinated with viable pollen.

### Example 7: Dwarf Female Grapevines with Parthenocarpic and Stenospermocarpic Seedless Fruit

Building on the findings from Example 6, the inventors developed dwarf plants that flower rapidly and produce seedless fruit even after pollination with viable pollen and methods for producing same. This was achieved by combining three genes in a single plant: 1) the mutated gene Gibberellic Acid Insensitive gene in either the heterozygous (GAI1/gai1) or homozygous state (GAI1/GAI1) that causes a dwarf stature and rapid flowering phenotype; 2) the female FSL locus (f/f in the homozygous state); and 3) the mutated locus of the Vitis vinifera MADS-box protein 5 (VvMADS5) gene (in either the heterozygous or homozygous state) that is associated with stenospermocarpy (SDL1) in grapevine. VvMADS5 had been previously isolated and the sequence deposited in GenBank database (GenBank: AF373604.1; Boss et al., (2002) J. Plant Sci., 162(6):887-895. This gene has been identified as a key gene associated with seedless-ness in several literature later and is also known as VviAGL11.

Briefly, pollen was collected in vials from seedless grape varieties during anthesis, dried in the oven at 42° C. overnight and stored in the freezer at −80° C. until use. The seedless varieties initially chosen were Crimson Seedless, Ruby Seedless, Black Gem Currant and MS-03-48-44. Later, pollen from varieties such as Fantasy seedless (FRESNO B 36-27×FRESNO C 78-68) and Summer Royal (FRESNO A 69-190×FRESNO C 20-149) have also been used to produce dwarf female grapevines with parthenocarpic and stenospermocarpic seedless fruit. When cap fall began in inflorescences of female microvine plants, all caps were gently removed and pollen from selected seedless varieties were brushed onto the stigmata of the flowers of dwarf female microvines developed in Example 6. Inflorescences were tagged with paper tags indicating the name of pollen donor and date of pollination. Pollination was repeated for the next one or two days to cover all the late developing flowers.

Fruit was harvested and seeds extracted around 3 months after pollination. The seeds were germinated and the segregating progeny were grown in pots in the glasshouse. When inflorescences developed in this segregating progeny, plants were crossed with viable pollen from a test variety such as Riesling to confirm the seedless-ness phenotype of the progeny and to identify and select truly seedless plants.

A seedless-ness marker test was also developed to confirm that those plants exhibiting a truly seedless phenotype had the VvMADS5 genotype. Briefly, primers were developed to isolate genomic regions of VvMADS5 with SNPS from seedless varieties such as Crimson Seedless and Ruby Seedless and seeded Sultana monococcus by PCR. Primers were then designed to enable genotyping of varieties for seedless-ness using KASP™ assay following the “Guide to running KASP™ genotyping reactions on the Roche LC480-series instruments” by LGC Biosearch Technologies. One set of primers (below) successfully identified the seedless-ness SNP and matched it with the phenotype. This marker is named as SDL1.

| | Primers used: |
|---|---|
| | VvSDLF1: |
| (SEQ ID NO: 20) | |
| | GAAGGTGACCAAGTTCATGCTATCCAGGCATTAGTTTCTCG |
| | VVSDLF2: |
| (SEQ ID NO: 21) | |
| | GAAGGTCGGAGTCAACGGATTATCCAGGCATTAGTTTCTCT |
| | VvSDLRev: |
| (SEQ ID NO: 22) | |
| | AAGTGGGTAGCCTGTGGAT |

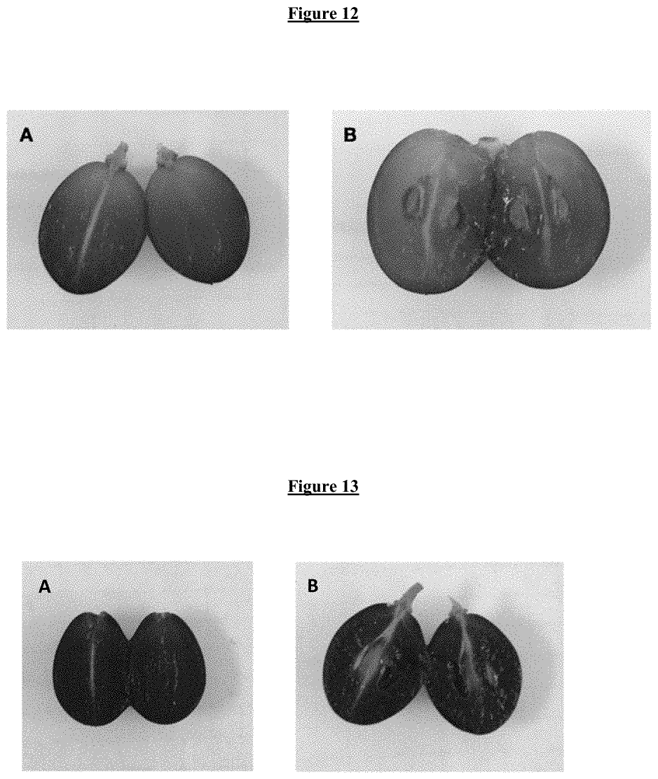

Scenarios exist where the inflorescences of female microvines may get pollinated by pollen in the air originating from other grapevines, in which case the berries may develop hard seeds. It is therefore important to develop truly seedless microvines which do not form hard seeds after pollination to account for circumstances where flowers are unintentionally pollinated by windblown pollen. The present inventors have achieved this by introducing the mutated VvMADS5 stenospermocarpy locus into female microvines comprising the mutant GAI1 dwarfing gene in combination with the FSL female (f/f) locus. Such plant produce seedless fruit even after pollination. When all three genes are combined, a dwarf grapevine is produced that has sterile pollen and produces seedless fruit with or without fertilisation. Genotyping with the seedlessness marker confirmed the mutated locus of the VvMADS5 gene. The phenotype has been further tested by artificially pollinating some inflorescences of several female microvines at the time of anthesis, with viable pollen from a genotype such as Riesling. Inflorescences were tagged with paper tags of the name of pollen donor and date of pollination. The pollinated inflorescences were allowed to grow into bunches and fruit harvested and sectioned to observe seed development (if any) around 3 months after pollination. Parthenocarpic seedlessness was evident in the berries that developed from unfertilized flowers (FIG. 13A). Stenospermocarpic seedlessness was evident in the berries that developed from pollinated flowers i.e., only soft seed traces that do not normally germinate were observed (FIG. 13B).

### Example 8: Dwarf Hermaphrodite Grapevines with Stenospermocarpic Seedless Fruit

The inventors also developed new dwarf hermaphrodite plants that flower rapidly and produce seedless fruit even after pollination with viable pollen, and methods of producing same. The plants and method combine two genes: 1) the mutated gene Gibberellic Acid Insensitive gene (in either the heterozygous (GAI1/gai1) or homozygous state (GAI1/GAI1) that causes a dwarf stature and rapid flowering phenotype; and 2) the mutated locus of the VvMADS5 gene (in either the heterozygous or homozygous state) that is associated with stenospermocarpy (SDL1) in grapevine.

When cap fall began in inflorescences of female microvine plants, all caps were gently removed and pollen from selected seedless varieties (as in Example 7) was brushed onto the stigmata of the flowers. Inflorescences were tagged with paper tags showing the name of the pollen donor and date of pollination. Pollination was repeated for the next one or two days to cover all the late developing flowers.

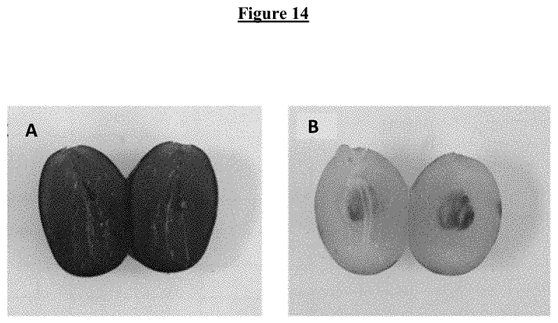

Fruit was harvested and seeds extracted around 3 months after pollination. The seeds were germinated and the segregating progeny were grown in pots in the glasshouse. Genotyping with the seedless-ness marker (described in Example 7) was used to confirm the mutated locus of the VvMADS5 gene in plants that produced hermaphrodite flowers and developed to give seedless berries. Berry sections of hermaphrodite microvines confirmed that plants that were genotyped to be stenospermocarpic seedless using the SDL marker and containing the mutated VvMADS5 locus were seedless or had only soft seed traces while other hermaphrodite plants had hard brown seeds (FIGS. 14A and B).

Hermaphrodite microvines exhibit typical hermaphrodite phenotype for the flowers and develop hard seeds in the berries. By introducing the mutated VvMADS5 locus into the hermaphrodite background (either by conventional plant breeding or recombinant DNA techniques), hermaphrodite micro vine plants with seedless berries have been produced. Seedless hermaphrodites are important for the table grape market and also for further breeding work to introduce seedless-ness into other grape genotypes.

### Example 9: Other Novel Combinations

Female and hermaphrodite microvines have been bred with new combinations of berry flesh colour (red flesh trait from grape variety Dunkelfelder) and berry flavor (muscat flavor from the grape varieties Muscat Gordo Blanco and Frontignac white; candy floss flavour from Muscadinia-MS27-31 hybrid). Thus a selection of microvines that can give year round production of fruit to suit different palates and taste have been developed.

### Example 10: Grape Berry Juice Production and Analyses

Analyses of berry juice from various microvine lines as exemplified herein using a f/f black berry seedless microvine (15C018V0005), a seedless white berry hermaphrodite line (15C018V0058) and a seeded Muscat flavoured hermaphrodite (17C001V0006). Analysis was performed using Oenofoss™ analyser for Brix (TSS) as per the manufacturer's instructions. The berries were crushed in a sterile plastic bag and 2 ml of must was transferred to a 2 ml Eppendorf tube and centrifuged for 1 minute at 13000 rpm. Approximately 0.6 ml of the supernatant was analysed using an Oenofoss™ analyser for Brix (TSS) to test sample pH, Total Acidity, Volatile Acidity, Alpha Amino Nitrogen, Ammonia, Tartaric Acid, Malic Acid and Density. Results are presented in Table 2.

### Example 11: Wine Production from Microvine Berries

Wine was made on a small scale from fruit produced from a seedless hermaphrodite (15C018V0058) and a seeded hermaphrodite (17C001V0006) microvine. 2 kg of grape bunches were transferred to a press seal bag (305 mm×405 mm 50 um) with a tablespoon of dry ice and 1.2 mL PMS 100 mg/ml solution (based on 50 ppm for 60% juice recovery). Berries were squeezed until all berries were disrupted and free running juice was visible. The juice was strained through a kitchen sieve and centrifuged for 2 minutes at 1489 ref to remove solids. 500 mL of juice was removed from the centrifuge bottle and transferred to a 500 ml Schott bottle with an airlock and silicon sampling septum. The juice was temperature adjusted for 1-2 hours for before adding yeast, DAP and PVPP. The juice was inoculated with 10 mL of an overnight yeast culture (Maurivin PDM Yeast), 1 mL of 476 mg/mL DAP stock (200 ppm YAN-Yeast assimilable nitrogen) and 1 mL of PVPP 130 mg/ml (260 ppm PVPP). The juice was fermented at 18° C. with 2 minute shaking at 100 rpm every two hours. Total sugars were measured every 24 hours. When the sugars reached 2.5 g/L the wine was racked by transferring into a clear wine bottle using a siphoning device under argon pressure with 500 μL Copper Sulphate 1 mg/mL and 500 μL PMS (100 mg/mL) and left to settle for 7 days at 4° C. The headspace was minimised by filling to the top with marbles. After cold settling, the wine was filtered through an 0.45 μm autoclaved ground water filter (Air-Met FTH-45) by using argon gas to push it into a combination of 200 mL, 100 mL and 50 mL amber bottles (Cospak). The bottles were sealed with Tampertell cap cello wadded caps (Cospak) and then sealed with wax. Finished wine analysis was performed for each grape variety wine sample using the Oenofoss™ analyser from Foss according to the manuctaurer's instructions.

### OenoFoss Measurements for Wine

Briefly, about 1 mL of wine was collected during wine making and transferred into 2 ml Eppendorf tube and centrifuged for 1 minute at 13000 rpm. Approximately 0.6 ml of the supernatant was analysed using an Oenofoss™ analyser for Ethanol, pH, Total Acidity, Volatile Acidity, Malic Acid, Wine Density and Glucose/Fructose.

Results of the wine analysis by OenoFoss™ is presented in Table 3.

### Discussion

The berries were picked before full maturity and so, sugar levels were sub-optimal for alcohol development. The resulting wine had an alcohol content below the detectable level of 8% by OenFoss. Nevertheless, the experiment successfully showed that the strong Muscat flavour was present in the wine prepared from the Muscat flavoured microvine line 17C001V0006. The seedless berries are likely to be useful for white wine production which currently requires the removal of seeds due to flavour problems arising from the seeds natural very high phenolics content. High phenolics can be extracted into the wine during the fermentation processing, therefore the absence of seeds may improve the wine quality. Skin-contact white wine, skin fermented white wine processing allows the wine to develop while the skin is still present unlike in conventional white wine production which crushes the grapes recovering the pressed juice into a fermentation vessel resulting in loss of colour pigments, phenols and tannins. Red wine requires skin contact and maceration for color, flavour and texture development.

TABLE 2
OenoFoss values for different characteristics of table grape juice (must) from
a sample of microvine grapes. Values are from randomly selected berry samples
of each line harvested at around 17-18 BRIX measured using a pocket refractometer

| | | | Total | | | |
|---|---|---|---|---|---|---|
| | | | Titratable | Malic | Tartaric | |
| | Plant ID/ | | Acid | acid | acid | |
| Phenotype | Line | pH | (g/L) | (g/L) | (g/L) | TSS |
| Female, | 15C018V0005 | 3.4491 | 9.591 | 5.055 | 5.273 | 18.564 |
| Black berry, | | | | | | |
| seedless | | | | | | |
| Hermaphrodite | 15C018V0058 | 3.73 | 9.2 | 5.7 | 4.2 | 14.6 |
| white berry, | | | | | | |
| seedless | | | | | | |
| Hermaphrodite | 17C001V0006 | 3.32 | 9.925 | 4.65 | 7.425 | 15.875 |
| Muscat flavour, | | | | | | |
| hard seeded | | | | | | |
| | | | Yeast | Alpha | | |
| | | Volatile | Assimilable | Amino | Gluconic | |
| | | Acids | N (YAN) | Nitrogen | acid | Ammonia |
| Phenotype | Density | (g/L) | (mg/L) | mg/L | (g/L) | (mg/L) |
| Female, | 1.1 | 0.0518 | 528.636 | 361.76 | 0.6 | 203.5091 |
| Black berry, | | | | | | |
| seedless | | | | | | |
| Hermaphrodite | 1.1 | 0.12 | 577.6 | 425.5 | 0.4 | 185.5 |
| white berry, | | | | | | |
| seedless | | | | | | |
| Hermaphrodite | 1.1 | 0.0325 | 294.775 | 184.18 | 0 | 134.9 |
| Muscat flavour, | | | | | | |
| hard seeded | | | | | | |

TABLE 3
OenoFoss analysis of small scale wine samples prepared from selected microvine grapes

| | | | | Titratable | Malic | Glucose/ | Volatile | |
|---|---|---|---|---|---|---|---|---|
| Microvine line | Phenotype | Ethanol % | PH | Acid (g/L) | Acid (g/L) | Fructose | Acids (g/L) | Density |
| 15C018V0058 | Hermaphrodite, | −999 | 3.29 | 10.5 | 4.6 | 1.6 | 0.19 | 0.9971 |
| | white berry, | | | | | | | |
| | seedless | | | | | | | |
| 17C001V0006 | Hermaphrodite, | −999 | 3.51 | 8.8 | 4.8 | 2.5 | 0.15 | 0.9963 |
| | Muscat flavour, | | | | | | | |
| | hard seeded | | | | | | | |

### Example 12: Genotypic and Phenotypic Evaluation of Homozygous T1 Mutants of VviFSL Generated by Gene Editing

### Position of the Guide Sequences in VviFSL

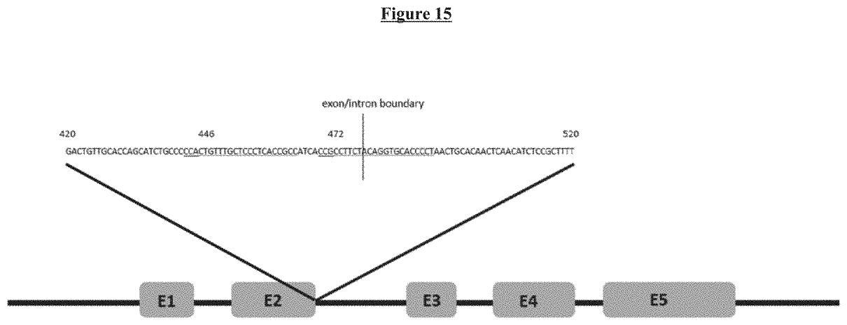

Two guide RNA sequences, FS1 and FS4, at the second exon of VviFSL were designed. These were chosen based on the presence of PAM sequences, their Benchling on target and off target scores and the ability to form CRISPR/Cas9 complex and cleave template DNA in vitro (data not shown). FIG. 15 shows the CRISPR/Cas9 vector and the cloning position for the guide RNA.

### Generation and Genotyping of T₁ Plants

Several T0 gene edited plants for both FS1 and FS4 guides were chosen for self-crossing to obtain T1 progeny, to determine inheritance patterns of the mutations and to obtain T1 homozygous mutants. T0 lines were selected for T1 progeny generation based on the mutation frequency. Self-crosses were performed and seeds germinated as described in Chaib et al., 2010. Roots from germinated embryos were genotyped using Amplicon Sanger sequencing (as per Example 5). Embryos were scored for being homozygous for a mutation, heterozygous or non-mutated homozygous and were transferred to SM medium for two weeks for plantlet formation and then potted into soil (BioGro soil mix purchased from Van Schaiks in Mt Gambier, South Australia) and transferred into a glasshouse or growth room for flower development for 4 months. The glasshouse temperature was set to 25° C. for day and 20° C. for night and watering was twice a day for 5 minutes. The growth room temperature was set at 25° C., humidity 85° C., 16 hour day light/8 hour night cycle light bulbs 400 W (420 kWh/1000 h) white light. The plants were water once a day for 5 minutes.

### Analysis of First Generation T0 Transgenic Plants for Gene Editing

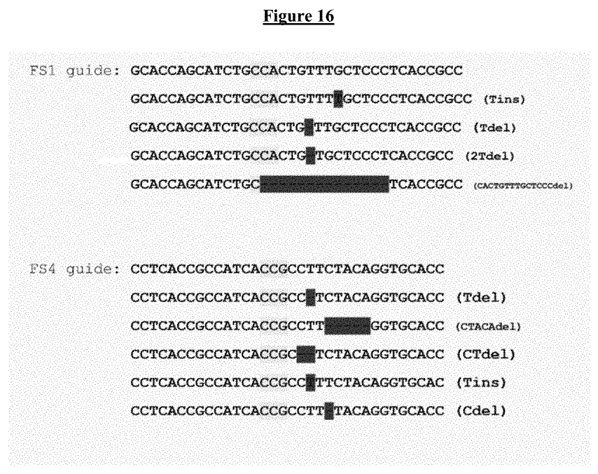

Fifteen GFP positive plants for both the FS1 and FS4 which were analyzed for CRISPR/Cas9 gene editing showed amplicon Sanger sequence disruption around the guide sequence. Nextgen sequencing analysis of these amplicons showed that gene editing frequency ranged from 91.8% to 35.9% for FS1 and 58.6% to 17.3% for FS4. Mutation types and locations where that occurred at a frequency of >10% in any one plant were identified. FIG. 16 shows these mutations. The mutations for both FS1 and FS4 mostly involved the base T and occurred 5 prime of the PAM site.

### Inheritance Patterns of Mutations in the T1 Generation

When the mutation frequency in the T0 parent was close to 100%, such as in Crosses A and E, it was likely that both alleles had been gene edited i.e., a bi-allelic mutation explaining why no wildtype progeny segregated in the T1 generation and there was 1:1 segregation for homozygous mutants and heterozygotes carrying different mutations on each allele. When the mutation frequency was around 50%, as for Crosses C, D Q, F & V, one of the alleles may have been gene edited and the other left alone i.e., a mono-allelic mutation. This gave rise to progeny where 50% were heterozygous and 25% homozygous mutants or 25% wildtype. This was the case for Crosses C, Q F and V showing insignificant Chi square p values for observed genotypes. Cross D, however, showed a significant deviation from the expected genotypic frequencies which indicated that the nextgen mutation frequency of 40% was not due to a monoallelic mutation, but due to the mutations existing in a chimeric state in the T0 plant where some segments were mutated and others not. T1 homozygous mutants were still obtained. Although no wildtype progeny where obtained for Cross A, it may be a low chimera because the flowers in the T0 parent remained hermaphrodite unlike for Cross E where T0 plants exhibited female flowers indicating that both FSL genes have been completely knocked out.

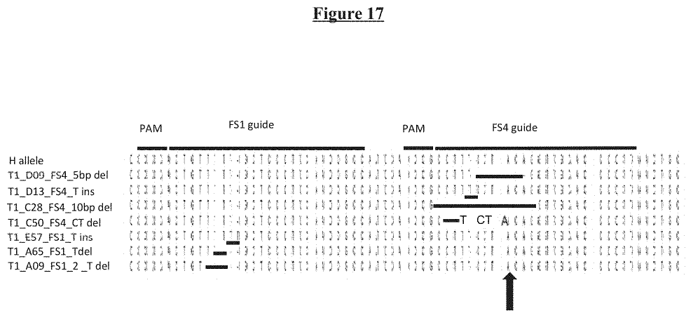

T1 homozygous mutants obtained from the T0 self crosses are listed in Table 4. Some of the mutations are the same for different crosses and they have been aligned in FIG. 17.

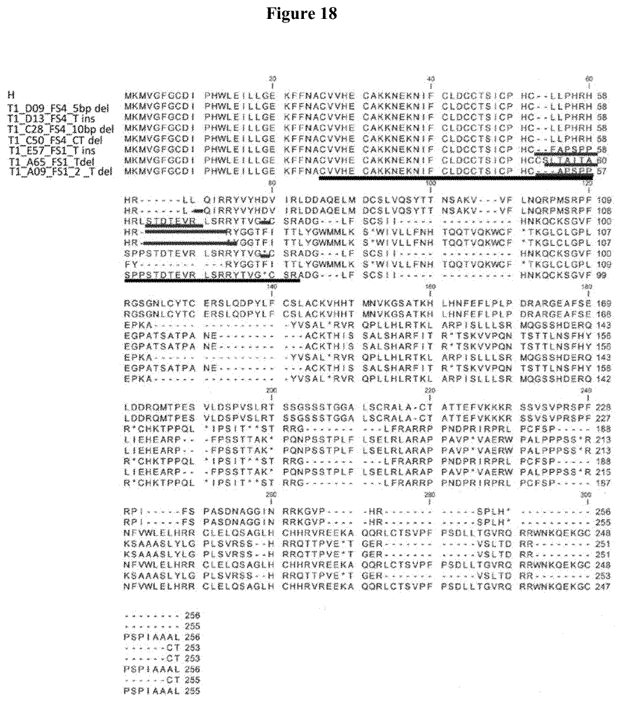

Homozygous T1 mutants were obtained for both FS1 and FS4 guide RNAs. The coding sequences were translated and aligned to determine the affect the mutations had on the protein sequence (FIG. 18).

TABLE 4
Mutation types obtained from each T0 cross. FS1
and FS4 refer to the original guide sequence.

| Cross | | T0 flower | Homozygous | |
|---|---|---|---|---|
| name | T0 plant | phenotype | mutation in T1 | T1 plant name |
| A | FS1_A1B_01 | hermaphrodite | FS1_2T_deletion | T1_A_FS1_2Tdel |
| C | FS4_M2B_14 | hermaphrodite | FS4_CT_deletion | T1_C_FS4_CTdel |
| C | FS4_M2B_14 | hermaphrodite | FS4_10bp_deletion | T1_C_FS4_10bpdel |
| D | FS4_M2B_03 | hermaphrodite | FS4_5bp_deletion | T1_D_FS4_5bpdel |
| D | FS4_M2B_03 | hermaphrodite | FS4_T_insertion | T1_D_FS4_Tins |
| E | FS1_BIA_3 | Female | FS1_T_insertion | T1_E_FS1_Tins |
| E | FS1_BIA_3 | Female | FS1_T_deletion | T1_E_FS1_Tdel |

### Mutations Cause Significant Amino Acid Sequence Changes

All DNA base deletions and inserting cause either a frameshift or nonsense mutation which could affect protein activity. T1_C_FS4_CTdel, T1-D_FS4, T1_A_FS1_tins and T1_A_FS1_2Tdel gave rise to a nonsense mutation within the PLATZ domain. T1_C-FS4_CTdel occurs earlier than the other mutants. T1_A_FS1_2Tdel and T1_AFs1_Tins give rise to a nonsense mutation at the same position.

### Flower Phenotype in the T1 Generation

All homozygous mutants from the T1 generation showed the conversion from hermaphrodite to female flowers with retracted stamens, and one mutant had no pollen production. Flowers from 2 to 6 individual inflorescences were scored and 22-54 flowers were scored for each mutant (Table 5). Pollen from all mutants showed viability using the pollen germination assay. This was further confirmed by using the pollen in crosses where the mutation was passed on. The pollen counts per anther for the mutants did not differ significantly between the original hermaphrodite plants and the mutants which produced pollen. The number of anthers analyzed ranged from 6-49. At least 3 individual homozygous mutants were confirmed to have female flowers and wildtype T1 progeny had the hermaphrodite phenotype confirming that the mutations were causing the phenotypic change.

TABLE 5
Genotype and phenotype summary for T1 Mutants.

| | | | | Mutation Position | | | | | |
|---|---|---|---|---|---|---|---|---|---|
| | | | | (From start codon | | | | | |
| | Guide | | T1 Homozygous | of FSL from | Flower | Flower | | Pollen | |
| T0 Plant | RNA | T1 plant name | Mutation | hermaphrodite V6) | Phenotype | Count | Inlf# | Count | Anther# |
| FS1_A1B_01 | FS1 | T1_A09_FS1_2Tdel | 2T deletion | 157bp | Female | 30/30 | 3 | 348.5 +/− | 7 |
| | | | | | flower | | | 79.5 | |
| FS4_M2B_14 | FS4 | T1_C72_FS4_CTdel | CT deletion | 182bp | Female | 54/54 | 5 | 0 | 49 |
| | | | | | flower and | | | | |
| | | | | | no pollen | | | | |
| FS4_M2B_14 | FS4 | T1_C74_FS4_10bpdel | 10bp deletion | 180bp | Female | 29/29 | 3 | 335.52 +/− | 21 |
| | | | | | flower | | | 156.6 | |
| FS4_M2B_03 | FS4 | T1_D18_FS4_5bpdel | 5bp deletion | 184bp | Female | 44/44 | 6 | 276.3 +/− | 16 |
| | | | | | flower | | | 82.0 | |
| FS4_M2B_03 | FS4 | T1_D13_FS4_Tins | T insertion | 184bp | Female | 22/22 | 2 | 283.5 +/− | 6 |
| | | | | | flower | | | 31.7 | |
| FS4_M2B_14 | FS4 | T1_C84_FS4_Tdel | T deletion | 184bp | Female | | | | |
| | | | | | flower | | | | |
| FS1_BIA_3 | FS1 | T1_E57_FS1_Tins | T insertion | 157bp from start codon | Female | | | | |
| | | | | | flower | | | | |
| FS1_A1B_01 | FS1 | T1_A47_FS1_Tdel | T deletion | 157bp from start codon | Female | | | | |
| | | | | | flower | | | | |

### Discussion

The inventors have demonstrated using CRISPR/Cas9 technology that VViFSL PLATZ transcription factor within grapevine linkage group 2 is necessary for normal male organ development in flowers. The knockout of the gene appears to be recessive as plants with only one mutated allele shows the hermaphrodite phenotype.

No differences were found at the DNA and protein level between VViFSL in the male and the hermaphrodite suggesting that it behaves similarly to Sp, the dominant gene necessary for male organ development described by Oberle 1938. In the female allele, however, amino acid substitutions along with an altered position for the start codon was found to render the protein non-functional and resulted in loss of male organ development.

The significantly lower expression in the female genotype (fsl/fsl) indicates that a lack of gene expression/protein amount level which interferes with male organ development. The ATG start of the female gene is further 5′ compared to the male or hermaphrodite sequence (FIG. 2) which could alter 5′ upstream sequences that can affect binding of transcription inducers. However, there are also amino acid substitutions present which could potentially influence protein activity.

With the CRISPR/Cas9 mutants, the inventors have similarly achieved the female phenotype by inactivating VviFSL as a result of changes in the protein sequence. The mono-allelic mutants of T0 maintained the original hermaphrodite flower phenotype indicating that only one functional FSL gene is necessary for male organ development making it a dominant trait.

The NCBI LOC100247272 appears to be the dominant FSL which gives rise to normal male organ development and LOC100852507 appears to be the recessive fsl which gives rise to abnormal male organ development according to the SNP. Indicating that the Pinot Noir genome is heterozygous for the sex locus FSL/fsl.

It is unclear why the CT mutation affected pollen fertility whereas the other mutations gave rise to the retracted stamens and viable pollen. The nonsense mutation occurs earlier on within the PLATZ domain and therefore could have more on an effect on its function.

This is the first time a PLATZ domain transcription factor has been described to have a role in flower development in plants.

### Example 13: Discussion

The present inventors have identified that a locus, termed the Flower Sex (FSL) locus, is responsible for flower sex in angiosperms, such as grapevines, and that different FSL locus genotypes and polypeptides expressed therefrom can be used to determine, control and/or select flower sex phenotype i.e., female, male or hermaphrodite flower phenotypes respectively. The inventors have characterized the locus responsible for male sex organ determination, the FSL locus in a Vitis sp., and have also demonstrated 100% concordance between female fsl/fsl and hermaphrodite FSL/FSL or FSL/fsl genotypes at a single nucleotide polymorphism (SNP) within a plant AT-rich sequence- and zinc-binding (PLATZ) domain of the FSL locus and the respective flower sex phenotype.

The present inventors have produced FSL knockout Vitis vinfera plants by introducing mutations in the PLATZ domain of the FSL locus using CRISPR. The inventors have shown that the resulting FSL knock out plants do not develop functional male reproductive organs, supporting the conclusion that expression the FSL locus is essential for development of functional male reproductive organs in flowers. Based on these findings, the present disclosure describes plants and plant parts with altered FSL polypeptide activity, as well as methods of producing plants with a particular flower sex phenotype by selecting for specific FSL locus genotypes or by modifying the FSL locus.

The present disclosure is also based on the finding by the inventors that new parthenocarpic seedless grape varieties may be produced by combining: (i) a FSL locus which determines male flower sex which has been modified to confer a female flower phenotype, and (ii) a polynucleotide which confers dwarf stature, such as a variant of the Gibberellic Acid Insensitive (GAI1) locus which confers dwarf stature and rapid flowering in grapevines. The inventors have shown that grapevines having the above fsl\\/GAI1 genetic profile produce parthenocarpic seedless fruit when flowers are unpollinated and fruit containing seeds when flowers are pollinated with viable pollen.

Furthermore, the present disclosure is based on the surprising finding by the inventors that new stenospermocarpic/parthenocarpic seedless grape varieties may be produced by combining: (i) a FSL locus which is homozygous in females or which has been modified to confer a female flower phenotype, (ii) a polynucleotide which confers dwarf stature, such as a variant of the GAI1 locus which confers dwarf stature and rapid flowering in grapevines; and (iii) a polynucleotide that confers stenospermocarpy, such as a variant of the Vitis vinifera MADS-box protein 5 (VvMADS5) locus. The inventors have shown that grapevines having the above fsl/GAI1/VvMADS5 genetic profile produce parthenocarpic seedless fruit when flowers are unpollinated and stenospermocarpic fruit when flowers are pollinated with viable pollen. In this regard, the present disclosure provides new “seedless-ness” genotypes which are capable of producing seedless fruit in grapes, including “true seedless” fruit even after pollination.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida and Allshire (2005) TRENDS Cell Biology 15: 251-258.
Antcliff (1980) Annales de l'Amelioration des Plantes 39:113-122.
Ausubel et al. (editors), Current Protocols in Molecular Biology, Greene Pub. (1988, including all updates until present), Associates and Wiley-Interscience.
Battilana et al. (2013) Molecular Biotechnology, 54(3):1031-1037.
Bevan et al. (1983) Nucleic Acid Research, 11: 369-385.
Bibikova et al. (2001) Molecular and Cellular Biology, 21: 289-287.
Bibikova et al. (2002) Genetics, 161:1169-1175.
Boss and Thomas, (2002) Nature, 416(6883):847-850
Bourque (1995) Plant Science, 105: 125-149.
Brown T. A. (editor), Essential Molecular Biology: A Practical Approach, (1991), Volumes 1 and 2, IRL Press.
Carmona et al. (2008) Journal of Experimental Botany, 59(10):2579-2596.
Certo et al. (2012) Nature Methods, 8:941-943.
Chaib et al. (2010) The Plant Journal, 62(6):1083-1092.
Chanderbali et al. (2010) PNAS USA, 107:22570-22575.
Christensen & Quail (1989) Plant Molecular Biology. 12:619-632.
Christensen et al. (1992) Plant Molecular Biology. 18:675-689.
Coito et al. (2017) Frontiers in Plant Science, 8:98.
Coito et al., (2019) PeerJ, DOI 10.7717/peerj.7879.
Coligan et al. (editors) Current Protocols in Immunology, John Wiley & Sons.
Comai et al. (2004) Plant Journal, 37: 778-786.
Coombe ‘Adoption of a system for identifying grapevine growth stages’ (1995) Aust. J. Grape and Wine Res. 1:104-110Dalbo, et al. (2000).
Doyon et al. (2010) Nature Methods, 7:459-460.
Doyon et al. (2011) Nature Methods, 8:74-79.
Fechter et al. (2012) Molecular Genetics and Genomics, 287 247-259.
Friedlander et al. (1977) Plant and Cell Physiology, 18(1):261-269.
Gelvin et al. Plant Molecular Biology Manual, Kluwer Academic Publishers, 1990
Glover and Hames (editors), DNA Cloning: A Practical Approach, (1995 and 1996) Volumes 1-4, IRL Press.
Groenen et al. (1993) Molecular Microbiology, 10:1057-1065.
Guo et al. (2010) Journal of Molecular Biology, 400:96-107.
Haft et al. (2005) Computational Biology, 1(6):e60 Harlow and Lane (editors) Antibodies: A Laboratory Manual (1988), Cold Spring Harbor Laboratory.
Hartmann and Endres, Manual of Antisense Methodology, Kluwer (1999).
Henikoff et al. (2004) Plant Physiology, 135: 630-636.
Hinchee et al. (1988) Biotechnology, 6: 915.
Hoe et al. (1999) Emerging Infectious Diseases, 5:254-263.
Iocco et al. (2001) Transgenic Research 10: 105-112.
Ishino et al. (1987) Journal of Bacteriology, 169:5429-5433.
Jackson (1992) In-situ hybridisation in plants. In: Molecular Plant Pathology: A Practical Approach. Practical Approach Series, 1 (85). Oxford University Press.
Janssen et al. (2002) OMICS Journal of Integrated Biology, 6:23-33.
Ji et al, (2015) Scientia Horticulturae, 193:367-373.
Ji et al. (2016) Journal of Integrated Plant Biology, 58:762-765.
Jie et al. (2017) Horticultural Plant Journal, 3(3):91-100.
Jinek et al. (2012) Science, 337(6096): 816-821.
Joshi (1987) Nucleic Acid Research, 15: 6643-6653.
Kim et al. (1996) PNAS USA, 93:1156-1160.
Kim et al. (2012) Genome Research, 22:1327-1333.
Koziel et al. (1996) Plant Molecular Biology, 32:393-405.
Last et al. (1991) Theoretical and Applied Genetics, 81(5):581-588.
Medberry et al. (1992) Plant Cell, 4: 185-192.
Medberry et al. (1993) Plant Journal, 3: 619-626.
Manzano et al. (2011) Plant Growth Regulation, 65:213-221.
Mao et al. (2017) Frontiers in Plant Science, 8:1291.
McElroy et al. (1990) Plant Cell, 2:163-171.
McConnell Smith et al. (2009) PNAS USA, 106:5099-5104.
Millar and Waterhouse (2005) Functional and Integrative Genomics, 5:129-135.
Miller et al. (2007) Nature Biotechnology, 25:778-785.
Mojica et al. (1995) Molecular Microbiology, 17:85-93.
Mojica et al. (2000) Molecular Microbiology, 36:244-246.
Nakata et al. (1989) Journal of Bacteriology, 171:3553-3556.
Nagano et al. (2001) Nucleic Acids Research, 29(20):4097-4105.
Negi and Olmo (1966) Science, 152:1624-1625.
Negi and Olmo (1971) Vitis, 9:265-279.
Odell et al. (1985) Nature, 313:810-812.
Oberle (1938) New York State Agricultural Experiment Station Bulletin 250:3-32.
Pannell (2017) Current Biology, 27(5):R191-R197.
Perbal (1984), A Practical Guide to Molecular Cloning, John Wiley and Sons J. (supp. 1987).
Pasquinelli et al. (2005) Current Opinions in Genetics and Development, 15: 200-205.
Peterson and Anhder, (1960) Science, 131: 1673-1674.
Pouwels et al. Cloning Vectors: A Laboratory Manual, 1985.
Ramirez et al. (2012) Nucleic Acids Research, 40:5560-5568.
Riaz et al. (2006) Theoretical and Applied Genetics, 113:1317-1329.
Royo et al. (2018) Plant Physiology, 177:1234-1253.
Sambrook et al. (1989) Molecular Cloning: A Laboratory Manual, Cold Spring Harbor Laboratory Press.
Senior (1998) Biotechology and Genetic. Engineering Reviews, 15: 79-119.
Sigrist, C. J. A., (2009) Nucleic Acids Research, 38:161-166.
Slade and Knauf (2005) Transgenic Research, 14: 109-115.
Smith et al. (2000) Nature, 407: 319-320.
So et al, (2015) POJ, 8(6):479-484.
Stalker et al. (1988) Science, 242: 419-423.
Szczepek et al. (2007) Nature Biotechnology, 25:786-793.
Taylor (1997) The Plant Cell 9:1245-1249.
Tanurdzic and Banks (2004) Plant Cell, 16(Suppl. 1):S61-S71.
Thillet et al. (1988) The Journal of Biological Chemistry, 263:12500.
van Embden et al. (2000) Journal of Bacteriology, 182:2393-2401.
Valleau (1916) The American Naturalist 50(597):554-564.
Velten et al. (1984) EPBO Journal, 3:2733-2730.
Waterhouse et al. (1998) PNAS USA, 95: 13959-13964.
Wang et al. (2012) Genome Research, 22:1316-1326.
Weissbach and Weissbach, Methods for Plant Molecular Biology, Academic Press, 1989.
Yamasaki et al. (2000) Plant and Cell Physiology, 41:608-616.
Yamasaki et al (2001) Plant and Cell Physiology, 42:608-619.
Yamasaki, et al (2005) Vitamins and Hormones, 72:79-110.
Zhang et al (2013) Current Biology, 23(20):1979-1989.

1. A plant or part thereof having an altered level of flower sex (FSL) polypeptide activity compared to a corresponding plant or part thereof having a FSL locus genotype which confers a male or hermaphrodite flower phenotype.

2. The plant or part thereof of claim 1, wherein:
an FSL locus genotype which confers a hermaphrodite flower phenotype comprises a hermaphrodite allele of the FSL locus encoding the FSL polypeptide comprising an amino acid sequence set forth in SEQ ID NO:1, a biologically active fragment thereof, or an amino acid sequence which is at least 40% identical to the sequence set forth in SEQ ID NO:1; and
an EST locus genotype which confers a male flower phenotype comprises a male allele of the FSL locus encoding the FSL polypeptide comprising an amino acid sequence set forth in SEQ ID NO:3, a biologically active fragment thereof, or an amino acid sequence which is at least 40% identical to the sequence set forth in SEQ ID NO:3.

3. The plant or part thereof of claim 1, wherein:
a hermaphrodite allele of the FSL locus encodes the FSL polypeptide comprising the amino acid sequence set forth in SEQ ID NO:1, or a biologically active fragment thereof; and
a male allele of the ESL locus encodes the polypeptide comprising the amino acid sequence set forth in SEQ ID NO:3, or a biologically active fragment thereof.

4. The plant or part thereof of any one of claims 1 to 3, comprising an FA locus comprising a polynucleotide sequence encoding the FSL polypeptide, where the polynucleotide sequence is modified relative to a corresponding polynucleotide sequence of a wildtype FSL locus allele which confers a male or hermaphrodite flower phenotype when expressed.

5. The plant or part thereof of claim 4, wherein a region of the polynucleotide sequence encoding a plant AT-rich sequence- and zinc-binding (KATZ) domain of the FSL locus is modified.

6. The plant or part thereof of claim 4 or 5, wherein the polynucleotide sequence encoding the FSL, polypeptide comprises one or more nucleotide additions, deletions or substitutions relative to the corresponding polynucleotide sequence of an ESL locus allele which confers a male or hermaphrodite flower phenotype when expressed.

7. The plant or part thereof of any one of claims 1 to 6, wherein ESL polypeptide activity is reduced in the plant or plant part relative to a level of ESL polypeptide activity in a corresponding wildtype plant or part thereof.

8. The plant or part thereof of any one of claims 1 to 7, wherein the altered activity of the FSL polypeptide causes a male reproductive part of a flower of the plant to be absent or non-functional.

9. A plant or part thereof which produces seedless fruit, said plant comprising:
(i) a polynucleotide that confers dwarf stature to a plant; and
(ii) a flower sex (FSL) locus which is homozygous for a female allele (f/f) conferring female flower phenotype.

10. The plant or part thereof of claim 9, wherein the FSL locus has an ORE which comprise a sequence set forth in SEQ ID NO: 5 or a sequence which is at least 70% thereto provided that the nucleotide corresponding to position 621 of the sequence set forth in SEQ ID NO: 5 is a A.

11. The plant or part thereof of any one of claims 1 to 8, comprising a polynucleotide that confers dwarf stature to the plant.

12. The plant or part thereof of any one of claims 9 to 11, wherein the polynucleotide that confers dwarf stature is altered relative to the corresponding wildtype polynucleotide sequence.

13. The plant or part thereof of any one of claims 9 to 12, wherein the polynucleotide that confers dwarf stature is a variant of the gibberellic acid insensitive (GAP) gene or a fragment thereof.

14. The plant or part thereof of claim 13, wherein the variant of the GAI1 gene or fragment thereof that confers dwarf stature to the plant comprises one or more mutations in the region encoding a DELLA domain.

15. The plant or part thereof of claim 13 or 14, wherein the variant of the GAP gene or fragment thereof is present in a homozygous (GAI1/GAI1) or heterozygous (GAI1/Gai1) state.

16. The plant or part thereof of any one of claims 13 to 15, wherein the GAI1 gene encodes a GAI1 protein comprising the amino acid sequence set forth in SEQ ID NO: 8 or an amino acid sequence which is at least 90% identical to the sequence set forth in SEQ ID NO:8 which retains the GA signalling function thereof, and wherein the variant of the GAI1 gene encodes a variant GAI1 protein comprising an amino acid sequence set forth in SEQ ID NO: 9 or an amino acid sequence which is at least 90% identical to the sequence set forth in SEQ ID NO:9 provided that the amino acid sequence of the variant GAIN protein comprises a Leu to His substitution at position 38 relative to the sequence set forth in SEQ ID NO: 8.

17. The plant or part thereof of any one of claims 13 to 15, wherein the GAI1 gene encodes a GAI1 protein comprising the amino acid sequence set forth in SEQ ID NO: 8 or an amino acid sequence which is at least 90% identical to the sequence set forth in SEQ ID NO:8 which retains the GA signalling function thereof, and wherein the variant of the GAI1 gene encodes a variant GAI1protein in which the DELLA domain is deleted, truncated or altered.

18. The plant or part thereof of any one of claims 9 to 17, wherein said plant produces parthenocarpic seedless fruit when flowers are unpollinated and fruit containing seeds when flowers are pollinated with viable pollen.

19. The plant or part thereof of any one of claims 9 to 18, comprising a polynucleotide that confers stenospermocarpy.

20. The plant or part thereof of claim 19, wherein the polynucleotide that confers stenospermocarpy is a variant of the Vitis vinifera MADS-box protein 5 (VvMADS5) locus.

21. The plant or part thereof of claim 20, wherein the VvMADS5 locus encodes a VvMADS5 protein comprising the amino acid sequence set forth in SEQ ID NO: 10 or an amino acid sequence which is at least 90% identical to the sequence set forth in SEQ ID NO:10 which retains the biological function thereof, and wherein the variant VvMADS5 protein comprises the amino acid sequence set forth in SEQ ID NO: 11 or an amino acid sequence which is at least 90% identical to the sequence set forth in SEQ ID NO:11 provided that the amino acid sequence of the variant VvMADS5 protein comprises an R197L substitution relative to the sequence set forth in SEQ ID NO: 10.

22. The plant or part thereof of claim 20, wherein the variant VvMADS5 locus comprises one or more mutations which results in deletion or truncation of the VvMADS5 protein.

23. The plant or part thereof of any one of claims 20 to 22, wherein the variant VvMADS5 locus Which confers stenospermocarpy is present in a homozygous or heterozygous state.

24. The plant or part thereof of any one of claims 19 to 23, wherein said plant produces parthenocarpic seedless fruit when flowers are unpollinated and stenospermocarpic fruit when flowers are pollinated with viable pollen.

25. A plant or part thereof which produces seedless fruit, said plant comprising:
(i) a flower sex (ESL) locus genotype Which is heterozygous for a female ESL locus allele and a hermaphrodite FSL locus allele (FSL/fsl), or homozygous for the hermaphrodite ESL, locus allele (FSL/FSL);
(ii) a polynucleotide that confers dwarf stature to a plant; and
(iii) polynucleotide that confers stenospermocarpy.

26. The plant or part thereof of any one of claims 1 to 25, wherein the plant is a dioecious plant species.

27. The plant or part thereof of any one of claims 1 to 25, wherein the plant is a hermaphroditic plant species.

28. The plant or part thereof of any one of claims 1 to 27, wherein the plant is a fruit producing plant.

29. The plant or part thereof of any one of claims 1 to 28, wherein the plant is a Vitis sp.

30. The plant or part thereof of any one of claims 1 to 29, wherein the plant part is a fruit, roots, stems, scion, cuttings, cells, seeds and seed parts.

31. A method of controlling flower sex in a plant, said method comprising altering a level of flower sex (FSL) polypeptide activity in the plant or part thereof compared to a level of FSL polypeptide activity in a corresponding plant or part thereof having an FSL locus genotype which confers a male or hermaphrodite flower phenotype.

32. The method of claim 31, wherein:
an FSL locus genotype which confers a hermaphrodite flower phenotype comprises a hermaphrodite allele of the ESL locus encoding the FSL polypeptide comprising an amino acid sequence set forth in SEQ ID NO:1, a biologically active fragment thereof, or an amino acid sequence which is at least 40% identical to the sequence set forth in SEQ ID NO:1; and
an FSL locus genotype which confers a male flower phenotype comprises a male allele of the FSL locus encoding the FSL polypeptide comprising an amino acid sequence set forth in SEQ ID NO:3, a biologically active fragment thereof, or an amino acid sequence which is at least 40% identical to the sequence set forth in SEQ ID NO:3.

33. The method of claim 32, wherein:
a hermaphrodite allele of the FSL locus encodes the FSL, polypeptide comprising the amino acid sequence set forth in SEQ ID NO:1, or a biologically active fragment thereof; and
a male allele of the FSL locus encodes the FSL polypeptide comprising the amino acid sequence set forth in SEQ ID NO:3, or a biologically active fragment thereof.

34. The method of any one of claims 31 to 33, wherein a plant or plant part having an altered level of FSL, polypeptide activity comprises an FSL, polypeptide comprising an amino acid sequence set forth in SEQ ID NO:2, a biologically active fragment thereof, or an amino acid sequence which is at least 40% identical to the sequence set forth in SEQ ID NO:2.

35. The method of any one of claims 31 to 34, comprising modifying a EST locus comprising a polynucleotide sequence encoding the FSL polypeptide or a biologically active fragment thereof.

36. The method of claim 35, comprising modifying a region within the FSL locus encoding a plant AT-rich sequence- and zinc-binding (PLATZ) domain.

37. The method of claim 335 or 36, comprising introducing one or more nucleotide additions, deletions or substitutions to the polynucleotide sequence encoding the FS1, polypeptide relative to a corresponding polynucleotide sequence of a wildtype FSL locus allele which confers a male or hermaphrodite flower phenotype when expressed.

38. The method of any one of claims 31 to 34, comprising introducing to the plant or plant part a RNA interference (RNAi) agent which targets a messenger RNA (mRNA) of the FSL locus or an allele thereof, thereby reducing FSL polypeptide activity in the plant or part thereof.

39. The method of any one of claims 31 to 38, wherein FSL, polypeptide activity is reduced in the plant or plant part relative a level of FSL polypeptide activity in a corresponding wildtype plant or part thereof.

40. The method of any one of claims 31 to 39, wherein reducing the activity of the FS1, polypeptide in the plant or plant part causes a male reproductive part of a flower of the plant or plant part to be absent or non-functional.

41. The method of any one of claims 31 to 40, wherein the plant is a dioecious plant species.

42. The method of any one of claims 31 to 40, wherein the plant is a hermaphroditic plant species.

43. The method of any one of claims 31 to 42, wherein the plant is a fruit producing plant.

44. The method of any one of claims 31 to 43, wherein the plant is a Vitis sp.

45. The method of any one of claims 31 to 44, wherein the plant part is selected from the group consisting of fruit, roots, stems, scion, cuttings, cells, seeds and seed parts.

46. A method of producing a plant that produces flowers of known sex, said method comprising the steps of:
i) crossing two parental plants,
ii) screening one or more progeny plants from the cross to determine the genotype at a flower sex (FSL) locus, and
iii) selecting a progeny plant capable of exhibiting a desired flower sex phenotype on the basis of the FS1, locus genotype, wherein
(a) an FSL locus genotype which is homozygous for a female ESL locus allele (f/f) confers a female flower phenotype,
(b) an ESL locus genotype which is heterozygous for a female allele and a hermaphrodite ESL locus allele (f/H) confers a hermaphrodite flower phenotype, and an FSL locus genotype which is homozygous for a hermaphrodite ESI, locus allele (H/H) confers a hermaphrodite flower phenotype,
(c) an FSL locus genotype which is heterozygous for a male ESL locus allele and either a female FA locus allele (M/f) or a hermaphrodite FSL locus allele (M/H) confers a male flower phenotype, and an FSL locus genotype which is homozygous for a male FSL locus allele (M/M) confers a male flower phenotype, thereby producing a plant which produces flower of known sex.

47. The method of claim 46, comprising selecting a progeny plant having an FSL locus genotype which is homozygous for a female allele (f/f) to thereby produce a plant which produces female flowers.

48. A method of producing a plant which produces seedless fruit, said method comprising the steps of:
i) crossing two parental plants, wherein one of the parental plants comprises a flower sex (ESL) locus which is homozygous for a female allele (f/f) conferring female flower phenotype, and the other parental plant comprises a polynucleotide that confers dwarf stature,
ii) screening one or more progeny plants from the cross for the presence or absence of the FSL locus which is homozygous for a female allele (f/f) and the presence or absence of the polynucleotide that confers dwarf stature, and
iii) selecting a progeny plant which comprises the FSL locus which is homozygous for a female allele (f/f) and which comprises the polynucleotide that confers dwarf stature,
thereby producing a plant which produces seedless fruit.

49. A method of producing a plant which produces seedless fruit, said method comprising the steps of:
i) crossing two parental plants, wherein at least one of the parental plants comprises (a) a flower sex (FSL) locus which is homozygous for a female allele (N) conferring a female flower phenotype, homozygous for FSL allele (FSL/FSL), or heterozygous for FSL (FSL/fsl) conferring a hermaphrodite flower phenotype, (b) at least one of the parental plants comprises a polynucleotide that confers dwarf stature, and (c) at least one of the parental plants comprises a polynucleotide that confers stenospermocarpy,
ii) screening one or more progeny plants from the cross for the presence or absence of the ESL locus which is homozygous for FSL (fsl/fsl), homozygous for a hermaphrodite allele (FSL/FSL), or heterozygous for a hermaphrodite allele and a female allele (FSL/fsl), (b) the presence or absence of the polynucleotide that confers dwarf stature, and (b) the presence or absence of the polynucleotide that confers stenospermocarpy, and
iii) selecting a progeny plant which comprises (a) an FSL locus genotype that confers a female or hermaphrodite flower phenotype, (b) a polynucleotide that confers dwarf stature, and (c) the polynucleotide that confers stenospermocarpy,
thereby producing a plant which produces seedless fruit.

50. The method of claim 49, wherein:
a progeny plant which comprises (a) an FSL locus genotype that confers a hermaphrodite flower phenotype, (b) the polynucleotide that confers dwarf stature, and (c) the polynucleotide that confers stenospermocarpy, produces stenospermocarpic seedless fruit; and
a progeny plant which comprises (a) an FSL locus genotype that confers a female flower phenotype, (h) the polynucleotide that confers dwarf stature, and (c) the polynucleotide that confers stenospermocarpy, produces parthenocarpic seedless fruit.

51. A method of producing a plant which produces parthenocarpic seedless fruit, said method comprising the steps of:
i) crossing two parental plants, wherein at least one of the parental plants comprises a flower sex (ESL) locus which is homozygous for a female allele (f/f) conferring female flower phenotype, at least one of the parental plants comprises a polynucleotide that confers dwarf stature, and at least one of the parental plants comprises a polynucleotide that confers stenospermocarpy,
ii) screening one or more progeny plants from the cross for the presence or absence of the ESL locus which is homozygous fsl/fsl which gives rise to female flower morphology, the presence or absence of the polynucleotide that confers dwarf stature, and the presence or absence of the polynucleotide that confers stenospermocarpy, and
iii) selecting a progeny plant which comprises the FSL locus which is homozygous for a female allele (f/f) the polynucleotide that confers dwarf stature, and the polynucleotide that confers stenospermocarpy,
thereby producing a plant which produces parthenocarpic seedless fruit.

52. The method of any one of claims 46 to 51, wherein the ESL locus or an allele thereof is as structurally defined in any one or more of the preceding claims.

53. The method of any one of claims 48 to 52, wherein the polynucleotide that confers dwarf stature is a variant of the gibberellic acid insensitive (GAR) gene or a fragment thereof as structurally defined in any one of the preceding claims.

54. The method of any one of claims 49 to 53, wherein the polynucleotide that confers stenospermocarpy is a variant of the Vitis vinifera MADS-box protein 5 (VvMADS5) locus as structurally defined in any one of the preceding claims.

55. The method of any one of claims 46 to 54, wherein the plant is a dioecious plant species.

56. The method of any one of claims 46 to 54, wherein the plant is a hermaphroditic plant species.

57. The method of any one of claims 46 to 56, wherein the plant is a fruit producing plant.

58. The method of any one of claims 46 to 57, wherein the plant is a Vitis sp.

59. Fruit of a plant of any one of claims 1 to 30 or of a progeny thereof, preferably wherein the plant is a Vitis sp.

60. A method of producing seedless fruit, the method comprising:
(i) growing a plant of any one of claims 1 to 30 to thereby produce fruit; and
(ii) optionally harvesting the fruit produced at (i); and
(iii) optionally processing the fruit harvested at (ii).

61. A product produced from a plant of any one of claims 1 to 30 or produced from a fruit thereof.

62. The product of claim 61, wherein the product is a food product, food ingredient, beverage product or beverage ingredient.

63. The product of claim 62, wherein:
(i) the food product is selected from the group consisting of table grapes, jam, marmalade, jelly, sultana, and raisins;
(ii) the food ingredient is vincotto, vinegar or grape must syrup (mosto cotta);
(iii) the beverage product is wine, grappa, brandy or grape juice;
(iv) the beverage ingredient is wine grapes or table grapes.

64. A flower sex (FSL) polypeptide comprising an amino acid sequence selected from the sequences set forth in SEQ ID NOs: 1, 2 or 3 or a biologically active fragment thereof, or an amino acid sequence which is at least 40% identical to a sequence set forth in SEQ ID NOs: 1, 2 or 3.

65. An isolated nucleic acid molecule encoding a flower sex (FSL) polypeptide, comprising (a) a polynucleotide sequence selected from the sequences set forth in SEQ ID NOs: 5-7 or a polynucleotide having an open reading frame (ORE) selected from the sequences set forth in SEQ ID NOs: 5-7 or (b) a polynucleotide sequence having at least 40% identity to one of the sequences set forth in SEQ ID NOs: 5-7 or a polynucleotide having an open reading frame (ORF) having at least 40% identity to one of the sequences set forth in SEQ ID NOs: 5-7; or (c) a polynucleotide sequence which is complementary to any of the polynucleotide sequences of (a) or (b).

66. An expression vector comprising the isolated nucleic acid molecule of claim 65 operably linked to a promoter.

67. A host cell comprising the nucleic acid molecule of claim 65 or an expression vector of claim 66.

68. The host cell of claim 67 which is a yeast, bacteria or plant cell.


SEQUENCE LISTING (22 sequences)

SEQ ID NO: 1 - protein, 255 aa, Vitis vinifera
MKMVGFGCDIPHWLEILLGEKFFNACVVHECAKKNEKNIFCLDCCTSICPHCLLPHRHHRLLQIRRYVYHDVIRLDDAQELMDCSLVQSYTTNSAKVVFLNQRPMSRPFRGSGNLCYTCERSLQDPYLFCSLACKVHHTMNVKGSATKHLHNFEFLPLPDRARGETFSELDDRQMTPESVLDSPVSLRTSSGSSSTGGALSCRALACTATTEFVKKKRSSVSVPRSPFRPIFSPASDNAGGINRRKGVPHRSPLH

SEQ ID NO: 2 - protein, 253 aa, Vitis
MVGFGCDIPHWLEILLGEKFFNACVVHECAKKNEKNIFCLDCCTSICPHCLLPHRHHRLLQIRRYVYHDVIRLDDAQKLMDCSLVQSYTTNSAKVVFLNQRPMSRPFRGSGNLCYTCDRSLQDPYLFCSLACKVHHTVNVKGGATKHLHNFEFLPLPDRARGEAFSELDDRQMTPESVLDSPVSLRTSSGSSPTGGAVSCRALACTATTEFVKKKRSSVSVPRFPFRPILSPASDNAGGINRRKGVPHRSPLH

SEQ ID NO: 3 - protein, 255 aa, Vitis sp.; identical to SEQ ID NO: 1

SEQ ID NO: 4 - DNA, 768 bp, Vitis vinifera
atgaaaatggttggttttggttgtgatattcctcactggcttgaaattcttctgggggagaagttctttaacgcgtgtgtggttcatgagtgcgctaagaagaacgagaagaacatcttttgcttggactgttgcaccagcatctgcccccactgtttgctccctcaccgccatcaccgccttctacagatacggaggtacgtttatcacgacgttatacggttggatgatgctcaagagctgatggattgttctcttgttcaatcatacacaacaaacagtgcaaaagtggtgtttttgaaccaaaggcctatgtctcggccctttagagggtccggcaacctctgctacacctgcgaacgaagcttgcaagacccatatctcttctgctctctcgcatgcaaggttcatcacacgatgaacgtcaaaggtagtgccacaaaacacctccacaactttgaattccttccattacctgatagagcacgaggcgaggccttttccgagctcgacgaccgccaaatgaccccagaatccgtcctcgactcccctgtttctctccgaacttcgtctggctcgagctccaccggcggtgccttgagttgcagagcgctggcctgcactgccaccaccgagttcgtgaagaaaaagcgcagcagcgtctctgtacctcggtcccctttccgtccgatcttctcaccggcgtcagacaacgccggtggaataaacaggagaaagggtgtccctcaccgatcgccgctgcactga

SEQ ID NO: 5 - DNA, 762 bp, Vitis sp.
atggttggttttggttgtgatattcctcactggcttgaaattcttctgggggagaagttctttaacgcgtgtgtggttcatgagtgcgctaagaagaacgagaagaacatcttttgcttggattgttgcaccagcatctgcccccactgtttgctccctcaccgccatcaccgccttctacagataaggaggtacgtttatcacgacgttatacggttggatgatgctcaaaagctgatggattgttctcttgttcaatcatacacaacaaacagtgcaaaagtggtgtttttgaaccaaaggcctatgtctcggccttttagagggtccggcaacctctgctacacctgcgaccgaagcttgcaagacccatatctcttctgctctctcgcatgcaaggttcatcacacggtgaacgtcaaaggtggtgccacaaaacacctccacaactttgaattccttccattacctgatagagcacgaggcgaggccttttccgagctcgacgaccgccaaatgaccccagaatccgtcctcgactcccctgtttctctccgaacttcgtctggctcgagccccaccggcggtgccgtgagttgcagagcgctggcctgcactgcaaccaccgagttcgtgaagaaaaagcgcagcagcgtctctgtgcctcggttccctttccgtccgatcctctcaccggcgtcagacaacgccggtggaataaacaggagaaagggtgtccctcaccgatcgccgctgcactga

SEQ ID NO: 6 - DNA, 768 bp, Vitis sp.; identical to SEQ ID NO: 4

SEQ ID NO: 7 - DNA, 768 bp, Vitis Sp.
atgaaaatggttggttttggttgtgatattcctcactggcttgaaattcttctgggggagaagttctttaacgcgtgtgtggttcatgagtgcgctaagaagaacgagaagaacatcttttgcttggactgttgcaccagcatctgcccccactgtttgctccctcaccgccatcaccgccttctacagatacggaggtacgtttatcacgacgttatacggttggatgatgctcaagagctgatggattgttctcttgttcaatcatacacaacaaacagtgcaaaagtggtgtttttgaaccaaaggcctatgtctcggccctttagagggtccggcaacctctgctacacctgcgaacgaagcttgcaagacccatatctcttctgctctctcgcatgcaaggttcatcacacgatgaacgtcaaaggtagtgccacaaaacacctccacaactttgaattccttccattacctgatagagcacgaggcgagaccttttccgagctcgacgaccgccaaatgaccccagaatccgtcctcgactcccctgtttctctccgaacttcgtctggctcgagctccaccggcggtgccttgagttgcagagcgctggcctgcactgccaccaccgagttcgtgaagaaaaagcgcagcagcgtctctgtacctcggtcccctttccgtccgatcttctcaccggcgtcagacaacgccggtggaataaacaggagaaagggtgtccctcaccgatcgccgctgcactga

SEQ ID NO: 8 - protein, 590 aa, Vitis vinifera
MKREYHHPHHPTCSTSPTGKGKMWDADPQQDAGMDELLAVLGYNVKASDMAEVAQKLEQLEEVIVNAQEDGLSHLASETVHYNPSDLSNWLGSMLSEFNPTPNCALDNPFLPPISPLDYTNCSTQPKQEPSIFDSPSLDYDLKAIPGKALYSHIEQPPQQPPAPPLYQRDNKRLKPTTSATANSVSSVIGGWGVPTESARPVVLVDSQETGIRLVHTLMACAEAVQQENLKLAEALVKQIGFLAVSQAGAMRKVATYFAEGLARRIYRLYPDKPLDSSFSDILQMHFYETCPYLKFAHFTANQAILEAFEGKKRVHVIDFSMKQGMQWPALMQALALRPGGPPSFRLTGIGPPSTDNTDHLHEVGWKLAQLAETIHVEFEYRGFVANSLADLDASMLELRDGESVAVNSVFELHSLLARPGGIERVLSAVKDMKPDIVTIVEQEANHNGPVFLDRFTESLHYYSTLFDSLEGCGVSPVNTQDKLMSEVYLGQQICNVVACEGPERVERHETLAQWRARLGSAGFDPVNLGSNAFKQASMLLALFAGGDGYRVEENNGCLMLGWHTRPLIATSAWQLANKPALPSSTPASN

SEQ ID NO: 9 - protein, 590 aa, Vitis vinifera
MKREYHHPHHPTCSTSPTGKGKMWDADPQQDAGMDELHAVLGYNVKASDMAEVAQKLEQLEEVIVNAQEDGLSHLASETVHYNPSDLSNWLGSMLSEFNPTPNCALDNPFLPPISPLDYTNCSTQPKQEPSIFDSPSLDYDLKAIPGKALYSHIEQPPQQPPAPPLYQRDNKRLKPTTSATANSVSSVIGGWGVPTESARPVVLVDSQETGIRLVHTLMACAEAVQQENLKLAEALVKQIGFLAVSQAGAMRKVATYFAEGLARRIYRLYPDKPLDSSFSDILQMHFYETCPYLKFAHFTANQAILEAFEGKKRVHVIDFSMKQGMQWPALMQALALRPGGPPSFRLTGIGPPSTDNTDHLHEVGWKLAQLAETIHVEFEYRGFVANSLADLDASMLELRDGESVAVNSVFELHSLLARPGGIERVLSAVKDMKPDIVTIVEQEANHNGPVFLDRFTESLHYYSTLFDSLEGCGVSPVNTQDKLMSEVYLGQQICNVVACEGPERVERHETLAQWRARLGSAGFDPVNLGSNAFKQASMLLALFAGGDGYRVEENNGCLMLGWHTRPLIATSAWQLANKPALPSSTPASN

SEQ ID NO: 10 - protein, 223 aa, Vitis vinifera
MGRGKIEIKRIENTTNRQVTFCKRRNGLLKKAYELSVLCDAEVALIVFSSRGRVYEYSNNNIKSTIDRYKKASSDSTNGGFTMEINAQYYQQESAKLRQQIQMLQNSNRHLMGDSLASLTVKELKQLENRLERGITRIRSKKHELLLAEIEYLQKREIELENESVYLRTKIAEVERLQQANMVSTHEFNAIQALVSRNFFQPNMIEGGSTGYPLHDKKVLHLG

SEQ ID NO: 11 - protein, 223 aa, Vitis vinifera
MGRGKIEIKRIENTTNRQVTFCKRRNGLLKKAYELSVLCDAEVALIVFSSRGRVYEYSNNNIKSTIDRYKKASSDSTNGGFTMEINAQYYQQESAKLRQQIQMLQNSNRHLMGDSLASLTVKELKQLENRLERGITRIRSKKHELLLAEIEYLQKREIELENESVYLRTKIAEVERLQQANMVSTHEFNAIQALVSLNFFQPNMIEGGSTGYPLHDKKVLHLG

SEQ ID NO: 12 - DNA, 35 bp, Artificial Sequence
  oligo dT B26
gactcgagtcgacatcgattttttttttttttttt

SEQ ID NO: 13 - DNA, 19 bp, Artificial Sequence
  CSFS1_CDS_F1
cagtgccagttttgcaggc

SEQ ID NO: 14 - DNA, 20 bp, Artificial Sequence
  FSL_RT_F1
acgccggtggaataaacagg

SEQ ID NO: 15 - DNA, 24 bp, Artificial Sequence
  FSL_RT_R1
tctcctttctccatccctaattga

SEQ ID NO: 16 - DNA, 20 bp, Artificial Sequence
  Guide FS1
ggcggtgagggagcaaacag

SEQ ID NO: 17 - DNA, 20 bp, Artificial Sequence
  Guide FS4
aggggtgcacctgtagaagg

SEQ ID NO: 18 - DNA, 20 bp, Artificial Sequence
  Guide FS2
gtcttgcaagcttcgttcgc

SEQ ID NO: 19 - DNA, 20 bp, Artificial Sequence
  Guide FS3
gcagcagcgtctctgtacct

SEQ ID NO: 20 - DNA, 41 bp, Artificial Sequence
  VvSDLF1
gaaggtgaccaagttcatgctatccaggcattagtttctcg

SEQ ID NO: 21 - DNA, 41 bp, Artificial Sequence
  VvSDLF2
gaaggtcggagtcaacggattatccaggcattagtttctct

SEQ ID NO: 22 - DNA, 19 bp, Artificial Sequence
  VvSDLRev
aagtgggtagcctgtggat